SEQUENCE LISTING (22 sequences)

SEQ ID NO: 1 - protein, 564 aa, Homo sapiens
  misc_feature: Incyte ID No 7484737CD1
MEKWDGNEGTSAFHMPEWMEIWLIDFHEYPASLMPDILLARSNPFHRGGSGAGNVTMLGSKKKYIVNGNSGIKAQIQFADQKQEFNKRPTKIGRRSLSRSISQSSTDSYSSAASYTDSSDDETSPRDKQQKNSKGSSDFCVKNIKQAEFGRREIEIAEQEMPALMALRKRAQGEKPLAGAKIVGCTHITAQTAVLMETLGALGAQCRWAACNIYSTLNEVAAALAESGFPVFAWKGESEDDFWWCIDRCVNVEGWQPNMILDDGGDLTHWIYKKYPNMFKKIKGIVEESVTGVHRLYQLSKAGKLCVPAMNVNDSVTKQKFDNLYCCRESILDGLKRTTDMMFGGKQVVVCGYGEVGKGCCAALKAMGSIVYVTEIDPICALQACMDGFRLVKLNEVIRQVDIVITCTGNKNVVTREHLDRMKNSCIVCNMGHSNTETDVASLRTPELTWERVRSQVDHVIWPDGKRIVLLAEGRLLNLSCSTVPTFVLSITATTQALALIELYNAPEGRYKQDVYLLPKKMDEYVASLHLPTFDAHLTELTDEQAKYLGLNKNGPFKPNYYRY

SEQ ID NO: 2 - protein, 178 aa, Homo sapiens [sequence omitted]

SEQ ID NO: 3 - protein, 371 aa, Homo sapiens
  misc_feature: Incyte ID No 2900469CD1
MLGSRAAGFARGLRALALAWLPGWRGRSFALARAAGAPHGGDLQPPACPEPRGRQLSLSAAAVVDSAPRPLQPYLRLMRLDKPIGTWLLYLPCTWSIGLAAEPGCFPDWYMLSLFGTGAILMRGAGCTINDMWDQDYDKKVTRTANRPIAAGDISTFQSFVFLGGQLTLALGVLLCLNYYSIALGAGSLLLVITYPLMKRISYWPQLALGLTFNWGALLGWSAIKGSCDPSVCLPLYFSGVMWTLIYDTIYAHQDKRDDVLIGLKSTALRFGENTKPWLSGFSVAMLGALSLVGVNSGQTAPYYAALGAVGAHLTHQIYTLDIHRPEDCWNKFISNRTLGLIVFLGIVLGNLWKEKKTDKTKKGIENKIEN

SEQ ID NO: 4 - protein, 384 aa, Homo sapiens
  misc_feature: Incyte ID No 6928818CD1
MAFPCRRSLTAKTLACLLVGVSFLALQQWFLQAPRSPREERSPQEETPEGPTDAPAADEPPSELVPGPPCVANASANATADFEQLPARIQDFLRYRHCRHFPLLWDAPAKCAGGRGVFLLLAVKSAPEHYERRELIRRTWGQERSYGGRPVRRLFLLGTPGPEDEARAERLAELVALEAREHGDVLQWAFADTFLNLTLKHLHLLDWLAARCPHARFLLSGDDDVFVHTANVVRFLQAQPPGRHLFSGQLMEGSVPIRDSWSKYFVPPQLFPGSAYPVYCSGGGFLLSGPTARALRAAARHTPIFPIDDAYMGMCLERAGLAPSGHEGIRPFGVQLPGAQQSSFDPCMYRELLLVHRFAPYEMLLMWKALHSPALSCDRGHRVS

SEQ ID NO: 5 - protein, 484 aa, Homo sapiens
  misc_feature: Incyte ID No 1801591CD1
MARLLRSATWELFPWRGYCSQKAKGELCRDFVEALKAVVGGSHVSTAAVVREQHGRDESVHRCEPPDAVVWPQNVEQVSRLAALCYRQGVPIIPFGTGTGLEGGVCAVQGGVCVNLTHMDRILELNQEDFSVVVEPGVTRKALNAHLRDSGLWFPVDPGADASLCGMAATGASGTNAVRYGTMRDNVLNLEVVLPDGRLLHTAGRGRHFRKSAAGYNLTGLFVGSEGTLGLITATTLRLHPAPEATVAATCAFPSVQAAVDSTVHILQAAVPVARIEFLDEVMMDACNRYSKLNCLVAPTLFLEFHGSQQALEEQLQRTEEIVQQNGASDFSWAKEAEERSRLWTARHNAWYAALATRPGCKGYSTDVCVPISRLPEIVVQTKEDLNASGLTGSIVGHVGDGNFHCILLVNPDDAEELGRVKAFAEQLGRRALALHGTCTGEHGIGMGKRQLLQEEVGAVGVETMRQLKAVLDPQGLMNPGKVL

SEQ ID NO: 6 - protein, 526 aa, Homo sapiens
  misc_feature: Incyte ID No 2257558CD1
MEPDPEPAAVEVPAGRVLSARELFAARSRSQKLPQRSHGPKDFLPDGSAAQAERLRRCREELWQLLAEQRVERLGSLVAAEWRPEEGFVELKSPAGKFWQTMGFSEQGRQRLHPEEALYLLECGSIHLFHQDLPLSIQEAYQLLLTDHTVTFLQYQVFSHLKRLGYVVRRFQPSSVLSPYERQLNLDASVQHLEDGDGKRKRSSSSPRSINKKAKALDNSLQPKSLAASSPPPCSQPSQCPEEKPQESSPMKGPGGPFQLLGSLGPSPGPAREGVGCSWESGRAENGVTGAGKRRWNFEQISFPNMASDSRHTLLRAPAPELLPANVAGRETDAESWCQKLNQRKEKLSRREREHHAEAAQFQEDVNADPEVQRCSSWREYKELLQRRQVQRSQRRAPHLWGQPVTPLLSPGQASSPAVVLQHISVLQTTHLPDGGARLLEKSGGLEIIFDVYQADAVATFRKNNPGKPYARMCISGFDEPVPDLCSLKRLSYQSGDVPLIFALVDHGDISFYSFRDFTLPQDVGH

SEQ ID NO: 7 - protein, 567 aa, Homo sapiens
  misc_feature: Incyte ID No 5701733CD1
MKPVWVATLLWMLLLVPRLGAARKGSPEEASFYYGTFPLGFSWGVGSSAYQTEGAWDQDGKGPSIWDVFTHSGKGKVLGNETADVACDGYYKVQEDIILLRELHVNHYRFSLSWPRLLPTGIRAEQVNKKGIEFYSDLIDALLSSNITPIVTLHHWDLPQLLQVKYGGWQNVSMANYFRDYANLCFEAFGDRVKHWITFSDPRAMAEKGYETGHHAPGLKLRGTGLYKAAHHIIKAHAKTWHSYNTTWRSKQQGLVGISLNCDWGEPVDISNPKDLEAAERYLQFCLGWFANPIYAGDYPQVMKDYIGRKSAEQGLEMSRLPVFSLQEKSYIKGTSDFLGLGHFTTRYITERNYPSRQGPSYQNDRDLIELVDPNWPDLGSKWLYSVPWGFRRLLNFAQTQYGDPPIYVMENGASQKFHCTQLCDEWRIQYLKGYINEMLKAIKDGANIKGYTSWSLLDKFEWEKGYSDRYGFYYVEFNDRNKPRYPKASVQYYKKIIIANGFPNPREVESWYLKALETCSINNQMLAAEPLLSHMQMVTEIVVPTVCSLCVLITAVLLMLLLRRQS

SEQ ID NO: 8 - protein, 318 aa, Homo sapiens
  misc_feature: Incyte ID No 2706884CD1
MLFSALLLEVIWILAADGGQHWTYEGPHGQDHWPASYPECGNNAQSPIDIQTDSVTFDPDLPALQPHGYDQPGTEPLDLHNNGHTVQLSLPSTLYLGGLPRKYVAAQLHLHWGQKGSPGGSEHQINSEATFAELHIVHYDSDSYDSLSELAERPQGLAVLGILIEVGETKNIAYEHILSHLHEVRHKDQKTSVPPFNLRELLPKQLGQYFRYNGSLTTPPCYQSVLWTVFYRRSQISMEQLEKLQGTLFSTEEEPSKLLVQNYRALQPLNQRMVFASFIQGSSYTTGRRGLKTERVWSSPQHKPRLRHKFLLRYHGCG

SEQ ID NO: 9 - protein, 556 aa, Homo sapiens
  misc_feature: Incyte ID No 4974616CD1
MRRFVYCKVVLATSLMWVLVDVFLLLYFSECNKCDDKKERSLLPALRAVISRNQEGPGEMGKAVLIPKDDQEKMKELFKINQFNLMASDLIALNRSLPDVRLEGCKTKVYPDELPNTSVVIVFHNEAWSTLLRTVYSVINRSPHYLLSEVILVDDASERDFLKLTLENYVKNLEVPVKIIRMEERSGLIRARLRGAAASKGQVITFLDAHCECTLGWLEPLLARIKEDRKTVVCPIIDVISDDTFEYMAGSDMTYGGFNWKLNFRWYPVPQREMDRRKGDRTLPVRTPTMAGGLFSIDRNYFEEIGTYDAGMDIWGGENLEMSFRIWQCGGSLEIVTCSHVGHVFRKATPYTFPGGTGHVINKNNRRLAEVWMDEFKDFFYIISPGVVKVDYGDVSVRKTLRENLKCKPFSWYLENIYPDSQIPRRYYSLGEIRNVETNQCLDNMGRKENEKVGIFNCHGMGGNQVFSYTADKEIRTDDLCLDVSRLNGPVIMLKCHHMRGNQLWEYDAERLTLRHVNSNQCLDEPSEEDKMVPTMQDCSGSRSQQWLLRNMTLGT

SEQ ID NO: 10 - protein, 598 aa, Homo sapiens
  misc_feature: Incyte ID No 70861047CD1
MASLRRVKVLLVLNLIAVAGFVLFLAKCRPIAVRSGDAFHEIRPRAEVANLSAHSASPIQDAVLKRLSLLEDIVYRQLNGLSKSLGLIEGYGGRGKGGLPATLSPAEEEKAKGPHEKYGYNSYLSEKISLDRSIPDYRPTKCKELKYSKDLPQISIIFIFVNEALSVILRSVHSAVNHTPTHLLKEIILVDDNSDEEELKVPLEEYVHKRYPGLVKVVRNQKREGLIRARIEGWKVATGQVTGFFDAHVEFTAGWAEPVLSRIQENRKRVILPSIDNIKQDNFEVQRYENSAHGYSWELWCMYISPPKDWWDAGDPSLPIRTPAMIGCSFVVNRKFFGEIGLLDPGMDVYGGENIELGIKVWLCGGSMEVLPCSRVAHIERKKKPYNSNIGFYTKRNALRVAEVWMDDYKSHVYIAWNLPLENPGIDIGDVSERRALRKSLKCKNFQWYLDHVYPEMRRYNNTVAYGELRNNKAKDVCLDQGPLENHTAILYPCHGWGPQLARYTKEGFLHLGALGTTTLLPDTRCLVDNSKSRLPQLLDCDKVKSSLYKRWNFIQNGAIMNKGTGRCLEVENRGLAGIDLILRSCTGQRWTIKNSIK

SEQ ID NO: 11 - protein, 230 aa, Homo sapiens
  misc_feature: Incyte ID No 7472794CD1
MLKSCFPESLKVYGAVMNINRGNPFQKEVVLDSWPDFKAVITRRQREAETDNLDHYTNAYAVFYKDVRAYRQLLEECDVFNWDQVFQIQKGPSPRLTYLSVANADLLNRTWSRGGNEQCLRYIANLISCFPSVCVRDEKGNPVSWSITDQFATMCHGYTLPEHRRKGYSRLVALTLARKLQSRGFPSQGNVLDDNTASISLLKSLHAEFLPCRFHRLILTPATFSGLPHL

SEQ ID NO: 12 - DNA, 1891 bp, Homo sapiens
  misc_feature: Incyte ID No 7484737CB1
atggagaagtgggacggtaatgagggcacctcagcttttcacatgcctgagtggatggaaatctggttgattgactttcatgagtatccagccagcttgatgcccgatattcttttggcaagaagtaatccttttcatagaggtggcagtggggctggtaatgtcactatgctgggcagcaagaagaaatacattgttaatggcaactctgggattaaggcccagatccagtttgctgaccagaagcaagaattcaacaaacgtcccaccaaaattggacgtcgctctttgtctcgttccatttctcagtcatctactgacagctacagctcagcggcttcatatacagatagctctgatgatgagacatcgcccagggacaagcagcaaaagaactctaagggaagcagtgacttctgtgttaagaacatcaaacaggcagagtttggacgaagagaaattgaaattgctgaacaagaaatgcctgcattgatggctttgaggaagagagctcaaggagaaaagcctttggctggagccaaaatcgtgggttgcacacacatcactgctcagactgctgtgcttatggaaactctgggtgctctgggggcccagtgccgatgggctgcctgcaacatctattccactctcaatgaagtggctgctgctctagcagaaagtggatttcctgtttttgcctggaagggagagtcagaagatgacttttggtggtgtatcgatagatgtgtgaatgtggagggctggcagccaaacatgatcttggatgatggaggggatcttacccactggatttataaaaagtatcccaacatgtttaagaaaatcaagggcatagtagaggagagtgttactggagttcacaggctgtaccaactgtccaaagctgggaagctgtgtgttccagccatgaatgtcaatgactcagtcaccaaacagaaatttgacaacctctactgttgccgtgaatcaattcttgatggacttaaaaggacaacagacatgatgtttggtggaaagcaagtggtagtctgtggctatggagaggtggggaaagggtgctgtgctgccctgaaagccatgggctccattgtgtatgtaactgaaattgaccccatctgtgccctgcaagcctgtatggatggatttcgactggtgaaattaaatgaggtcatccgacaagtggacattgttattacctgtacaggtaacaagaatgtggtaaccagagagcacttggaccgtatgaagaatagctgcatcgtttgtaacatgggacattccaacacagagactgacgtggcgagtctgcggacaccagaactgacctgggagcgagtgagatctcaagttgaccatgtgatatggcctgatggcaagaggatagtactgctggcagagggccgcctgctgaaccttagctgctccacagtgcctacatttgtgctctcaatcactgctactactcaggctcttgccttgatagagctttacaatgctcctgagggtcgctataagcaggatgtctacctgttgcccaagaagatggatgagtatgtggccagcctacacctgcctacctttgatgcccacttgacagagctgacagatgaacaggccaagtatctgggactcaacaagaatgggcccttcaagcctaattactacaggtattaagttcctgtaactcaaaccagaatttttaaggaatagaactccaagccttttctccactactatacaagaaagaattcagcaagctgcttctccaatcaaagctgcctgccgtgctcaccctgtgtgttaggttatttatttattaaaatcaagaatcctgtgcctgcaaaaaaaaaaaaaaaaaaaaaaaaaaaaa

SEQ ID NO: 13 - DNA, 1056 bp, Homo sapiens
  misc_feature: Incyte ID No 7485242CB1
agatagtaatgcctctttcctcccacccatccaaccctcttagcacaggggctcatcaaactcggggacattccctcctcccagactgatttttacctagaaacctttctagcctctcctcatctcttgactgtcttggccttggcaagaagaatacacgtccctacccagctatcttcagctcctgtccttcctcccagctgccagagaattgtcaggtcagcctgctccttctttcttctctgcattttttactagcaggagtgccaggggcccaaggactcttgggaagagtctctcacccaaggaccagttagagctcttattctgcaaatgtagctcacggactctgcagaggctgagattctgtggtggaccaggggagcagggctggctgaaggtggctggttgtctgggtggccttgggcaagcatctcctctttctgggtcatgagaacttggttatgggtgagatgatctctaaagtactgttgggttgggaatgctggaggctctgggatgtggcagacagccacttcaggcccactgctgcccccaaccaccttaactctaccagcaggaggaatggcaccagctgtgacccggctccttttcctccagcttgttctggggccaactctggtcatggacatcaagatgcagattggcagcaggaacttctataccttaagcattgactatcccagggttaactacccaaagggtttccggggctattgtaatggtctgatgtcctatatgcgaggcaagatgcaaaattcagattgcccaaagatccattatgtgatacatgccccttggaaggccatccagaagttctgcaagtatagtgacagcttctgtgagaattacaatgaatactgcacactcacccaggattccctccccatcacggtctgctccctgagccaccaacagccacccactagctgctactacaatagcaccctaaccaaccagaagctctacctactctgctcccgcaagtatgaagctgatccaataggtatcgctggtctctattcgggaatttaa

SEQ ID NO: 14 - DNA, 1520 bp, Homo sapiens
  misc_feature: Incyte ID No 2900469CB1
gtcccggcctcaccagcgccatgctgggctcgcgagccgcggggttcgcgcggggcctgcgggctttggcactggcgtggctgccgggctggcggggccgctccttcgccctggcgcgtgcggcaggcgcgccccacggtggtgacttgcagccccccgcctgtcccgagccgcgcgggcgccagctcagtttgtccgcggcggcggtggtggactctgcgccccgccccctgcagccgtacttgcgcctcatgcggttggacaagcccattggaacctggcttctgtatttaccatgtacctggagcattggtttggcagctgaaccaggttgttttccagattggtacatgctctccctctttggcactggagctattctgatgcgtggagcaggctgtactattaatgacatgtgggaccaggactatgataaaaaggttacaagaacagccaatcgtccaatagccgctggagacatttcaacttttcagtcctttgtttttcttgggggacagctaaccctggcactgggtgttcttctgtgtctaaattactacagtatagctctgggagcaggatccttacttcttgtcatcacctacccactaatgaaaagaatttcatactggcctcaactagccctgggcttgacatttaattggggagcgttacttggatggtctgctatcaagggttcctgtgatccatctgtttgcctgcctctttatttttctggagttatgtggacactaatatatgatactatttatgcccatcaggacaaaagagatgatgttttgattggtcttaagtcaacggctctgcggttcggagaaaataccaagccgtggctcagtggcttcagtgttgcaatgctgggggcactgagcctagtgggtgtgaacagtggacagactgctccctactacgctgccctgggtgctgtaggagcccatctgactcaccagatttacactctagacatccacagacctgaggattgttggaataaatttatctccaaccgaacactgggactaatagtttttttagggattgtccttgggaatttgtggaaagaaaagaagacagacaaaacaaagaagggtatagagaataaaatagaaaattaatgaatgaaatttatctaggaatttttaaaacattttttacaaaatataattagatttgaatacaaaatctgatacaatatgttaaagaattaagaacctgaagatgaagatttagagcatatttacctggattttacttatttgctagcaaaattcccccttgtcacagaaaccagggactcttcaggatttgagatggccttgagtattttagttgatacattcttctgcccattataattctcacctgaagttatggggattgcacgggttttggcactttagaaaaagcctgatgtgggtcttacataaatgaatgtctgtataagaaaatggactcttttttttagggaaaaataaaagcaactatgggaaaaaaaaaaa

SEQ ID NO: 15 - DNA, 3007 bp, Homo sapiens
  misc_feature: Incyte ID No 6928818CB1
gcaaagggaactgagaaacatggtcaaaaagaaaggaggaaaggcaggagagcgtagagtctgttttaaggagggagaaggaacggggcacagtggctcatgcctgtaatcccagcacttttcggggctgaggtgggaggatcacttgaacccaggagttcaagaccagactgggcaatatagtgggaacccgtctctacagaaaaaaaaaaaaaaatatttaatggacccaggtgtgtggtgggcacgctcctggtagagcgcctcaactaactccaaaagctggaagtggggaggactggcttgagcctaaggtgggtcaagactggcagtggagtcatggatgggtggtcactggtactccaggcctggggtggacacaggcaagagaccctggtctcaaattaacataacataggccaaccgggtctggaagatggcaagaacctgatgtgactgaatttttagcagggcacagttaaatacgggaaaggttgttctgacctgggattcgtcctgggggtcttcataaaagccaggcctgaggtgcacctgggcaggctacatgcaggagagggcctgaaggtgaatgtttgggaacagcaaataataccagcaggtagaatgaggaggcagagctagaggatgccgtggagatcatctcacccataaggtcatatcacccgtgtcacccagtcactggccacttcatactcatttcatgctcacaacagccctgcagtaggaattcactaacccttcgtcacagatgagaacactcagacccagccatcggaactgactgttcagacggtgcagatggggaagtggggatggcagaattcggctctgggtctgagttcagtgctatcacctcatgcttcctcgactcgggaggcaacgaggggaagtgattagagatgatccgagctggacagaggcggagaggagggaataaatgagagggatgtagggcgcagaatcaacaggatgcggagagcaggtagcagctgagaaggacaaggaagaaggctggggcttctggctggagtagtcaggtgaacggaggtgggaacagcagggtatgggttcaggggaagatggagactcagacgatgagtccactgggcctatgtgacgggttgtggggcggtgttgtcaggaggcaaagagagactcaaatctgaaggtcagaggcacggtccggagtgtgggagaccagagaccgtcagtgtaatcttggattttgattcctttttaccacttaacagcagagttgagaaggggctggagctgggttctgggagagaagtgacggggtacgggtggtgtcctgccggtgtcaaagacgacttccggctcacctctgactcggtttcccccacagatggcttttccctgccgcaggtccctgactgccaagactctggcctgcctcctggtgggcgtgagtttcttagcactgcagcagtggttcctccaggcgccaaggtccccgcgggaggagaggtccccgcaggaggagacgccagagggtcccaccgacgctcccgcggctgacgagccgccctcggagctcgtccccgggcccccgtgcgtggcgaacgcctcggcgaacgccacggccgacttcgagcagctgcccgcgcgcatccaggacttcctgcggtaccgccactgccgccacttcccgctgctttgggacgcaccggccaagtgcgccggcggccgaggcgtgttcctgctcctggcggtgaagtcggcgcctgagcactacgagcgacgcgagctcatccggcgcacgtgggggcaagagcgcagctacggcgggcggccagtgcgccgcctctttctattgggcaccccgggccccgaggacgaggcgcgcgcggagcggctggcggagctggtggcgctggaggcgcgcgagcacggcgacgtgctgcagtgggccttcgcggacaccttcctcaacctcacgctcaagcacctgcacttgctcgactggctggctgcacgctgcccgcacgcgcgctttctgctcagcggcgacgacgacgtgttcgtgcacaccgccaacgtagtccgcttcctgcaggcgcagccacccggccgccacctgttctccggccagctcatggagggctccgtgcccatccgcgacagctggagcaagtacttcgtgccgccgcagctcttccccgggtccgcttacccggtgtactgcagcggcggcggcttcctcctgtccggccccacggcccgggccctgcgcgcggccgcccgccacaccccgatcttccccatcgacgacgcctacatgggcatgtgtctggagcgcgccggcctggcgcccagcggccacgagggcatccgacccttcggcgtgcagctgcctggcgcacagcagtcctccttcgacccctgcatgtaccgcgagttgctgctagtgcaccgcttcgcgccctacgagatgctgctcatgtggaaggcgctgcacagccccgcgctcagctgtgaccggggacaccgggtctcctgaggccagttgggcggcttcagccccgggcctccaaccatgtccatgctgagaaggcagctttcccgctctgggtaccttacgtcctgcccagctctgtgcacctgaaccccagctgcgcactgaaatcagctggggtggggggtgtggaaaatgcctacatcctggctccatctcccgaagtttcgatttgattagtctggggtggacccagacatgttaagtattttttaagttcctccagtgatgcgaatgtgcagctaggcctgaggaccactcggctagactatctcttcatcctcgcaaagccagctccaccgcctctctgcaagaattccgggcccctcgctcccacactcgggtcctcttgagcagtggagcaagggagacctgggagcgtgggagccaggatcagggcccctgcatgtgctacaaatgtcagttgtgattccactgttacacgtgatg

SEQ ID NO: 16 - DNA, 2058 bp, Homo sapiens
  misc_feature: Incyte ID No 1801591CB1
gctgccacagtagctgactggtcaccctgccacccagtgctcacaccctctggccagtgcctggctatggcccgactgctcaggtctgcaacctgggagctgttcccctggaggggctactgctcccagaaggcaaagggagagctctgcagggacttcgtagaggctctgaaggccgtggtgggcggctcccacgtgtccactgccgcggtggtccgagagcagcacgggcgcgatgagtcggtgcacaggtgcgaacctcctgatgctgtggtgtggccccagaacgtggagcaggtcagccggctggcagccctgtgctatcgccaaggtgtgcccatcatcccattcggcaccggcaccgggcttgagggtggcgtctgtgctgtgcagggcggcgtctgcgttaacctgacgcatatggaccgaatcctggagctgaaccaggaggacttctctgtggtggtggagccaggtgtcacccgcaaagccctcaacgcccacctgcgggacagcggcctctggtttcccgtggacccaggcgcggacgcctctctctgtggcatggcggccaccggggcgtcggggaccaacgcggtccgctacggcaccatgcgggacaacgtgctcaacctggaggtggtgctgcccgacgggcggctgctgcacacggcgggccgaggccggcatttccggaagagtgcagccggctacaacctcacggggctcttcgtgggctccgaggggacgctgggcctcatcacagccaccaccctgcgcctgcaccctgcccctgaggccacagtggccgccacgtgtgcgttccccagtgtccaggctgctgtggacagcactgtacacatcctccaggctgcagtgcccgtagcccgcattgagttcctggatgaagtcatgatggatgcctgcaacaggtacagcaagctgaattgcttagtggcgcccacactcttcctggagttccatggctcccagcaggcactggaggagcagctgcagcgcacagaggagatagtccagcagaacggagcctctgacttctcctgggccaaggaggccgaggagcgcagccggctttggacagcacggcacaatgcctggtacgcagccctggccacgcggccaggctgcaagggctactccacggatgtgtgtgtgcccatctcccggctgccggagatcgtggtgcagaccaaggaggatctgaatgcctcaggactcacaggaagcattgtcgggcatgtgggtgacggcaacttccactgcatcctgctggtcaaccctgatgacgccgaggaactgggcagggtcaaggcttttgcagaacagctgggcaggcgggcactggctctccacggaacgtgcacgggggagcatggcatcggaatgggcaagcggcagctgctgcaggaggaggtgggcgccgtgggcgtggagaccatgcggcagctcaaggccgtgctagacccccaaggcctcatgaatccaggcaaagtgctgtgaagggggtctgagcacttagcccacaagttccctgactacggagccggttctggaacttttcttcatgccacggcccctgcaaggaaatagatgctgaggcagtcttcctgccagcgagcccactgtatctgggcccaaggccagagggcccagagagaagcctgagcaccgtgttacctccctggccctctggctggccccaggagcctttggttcagtaaacgacccagggtggttcccagcaaagctgcttcctctctgctcctacgcatcctgtcctggcgggaagagagcgtctgggtccattcaagactctgatgacacccctccccgaggcctcccactgccggggtcccaggacccttcccccttcacctggtgacaggaacactcctttcctggtatggaacgtgagctcccgtgacatgatgataggtcttctccttggggcctcccccaataaatctgtaataaacctgaaacccacctacagctacctggaagtcagcaggcagcacttcctcccctccccctcca

SEQ ID NO: 17 - DNA, 1951 bp, Homo sapiens
  misc_feature: Incyte ID No 2257558CB1
gcggcgcgcgcaggtcaggcggcggcgggatggagcccgatcccgagcccgcggccgtggaggttcccgcggggcgcgtgctcagcgcccgggagctcttcgccgcccgctcgcggtcgcagaagctgccccagcgctcgcatggccccaaggactttctgcccgacggctcggcagctcaggccgagcggctgcgccggtgccgggaagagctctggcagctgctggcagagcagcgcgtggagcgcctgggcagcttggtggctgccgagtggaggccagaagagggcttcgtggagttgaagtctcccgcgggcaaattctggcagaccatgggcttctcagagcagggccggcagcgccttcacccggaagaggccttgtatcttctggagtgtggctccatccacctcttccaccaagacctgccactgtctatccaggaagcttaccagctgctgctgaccgaccacactgtgaccttcctgcagtaccaggtcttcagccacctgaagaggttgggttatgtggttcgacgattccaaccaagctctgtcctgtccccgtatgagaggcagcttaacctggatgccagcgtgcagcacttggaggatggagatggcaagagaaagaggagcagctccagccctcggtccattaataagaaggccaaggccctggacaactccctgcaacccaagagtctggcagcctccagcccacctccctgcagccagcccagccaatgcccagaggagaaaccccaggagtcaagccccatgaagggcccagggggcccctttcagcttctggggtccctgggccccagccctggcccggccagggagggggtggggtgcagctgggagagtggcagagccgagaacggagtcacgggagccggtaagcggcgctggaacttcgagcagatctccttccccaacatggcttcagacagccgccacacccttctgcgcgccccagccccagagctgctcccggccaacgtggctgggcgggagacagacgctgagtcctggtgccagaagctgaaccagcgcaaggagaagctctccaggcgggaacgggagcaccacgcggaggccgcgcagttccaggaagatgtcaacgccgatcccgaggtgcagcgctgctccagctggcgggagtacaaggagctgctgcagcggcggcaggtgcagaggagccagcgccgggcccctcacctgtggggccagcccgtcaccccgctgctgagtcctggccaggccagctccccagccgtggtccttcagcatatctctgtgctgcagacaacacaccttcctgatggaggtgcccggctgttggagaagtctgggggcttggaaatcatctttgatgtttaccaggccgacgctgtggccacattccgaaagaataaccctggcaaaccctatgcccggatgtgcattagtggatttgatgagcctgtcccagacctctgcagcctcaagcggttgtcttaccagagtggggatgtccctctgatctttgccctggtggatcatggtgacatctccttctacagcttcagggacttcacgttgccccaggatgtggggcactgacctcacagctctgcagaggatggagcttgctccgggggaccgggactgtctgttctcagggaccatctcggctgcctcctgtacccagactctaacctgtagcttcagaggccagtctgggccttggccctgggtgtctgatactcacagagtgaaactgtgaccctctcccttccctgctgccttgcagtgacccctctggaactcaggactcgattttaaggacccaggaggtggggcagaagagaggactgtgtgcctttaacgagagggtgcctgcttcgtgctataaagccaaagccattaaaaatagatttcttttctgcaaaaaaaaaaaaaaa

SEQ ID NO: 18 - DNA, 2266 bp, Homo sapiens
  misc_feature: Incyte ID No 5701733CB1
ggctgtgctgtggctcctctctcaggggacagcgtaggccctgggggtcagttggagagggctctgactaccagcgactgctctgggggtgtctgcgatcaaggacgatcctgggtatgggggagggccaggcaccatgaagccagtgtgggtcgccacccttctgtggatgctactgctggtgcccaggctgggggccgcccggaaggggtccccagaagaggcctccttctactatggaaccttccctcttggcttctcctggggcgtgggcagttctgcctaccagacggagggcgcctgggaccaggacgggaaagggcctagcatctgggacgtcttcacacacagtgggaaggggaaagtgcttgggaatgagacggcagatgtagcctgtgacggctactacaaggtccaggaggacatcattctgctgagggaactgcacgtcaaccactaccgattctccctgtcttggccccggctcctgcccacaggcatccgagccgagcaggtgaacaagaagggaatcgaattctacagtgatcttatcgatgcccttctgagcagcaacatcactcccatcgtgaccttgcaccactgggatctgccacagctgctccaggtcaaatacggtgggtggcagaatgtgagcatggccaactacttcagagactacgccaacctgtgctttgaggcctttggggaccgtgtgaagcactggatcacgttcagtgatcctcgggcaatggcagaaaaaggctatgagacgggccaccatgcgccgggcctgaagctccgcggcaccggcctgtacaaggcagcacaccacatcattaaggcccacgccaaaacctggcattcttataacaccacgtggcgcagcaagcagcaaggtctggtgggaatttcactgaactgtgactggggggaacctgtggacattagtaaccccaaggacctagaggctgccgagagatacctacagttctgtctgggctggtttgccaaccccatttatgccggtgactacccccaagtcatgaaggactacattggaagaaagagtgcagagcaaggcctggagatgtcgaggttaccggtgttctcactccaggagaagagctacattaaaggcacatccgatttcttgggattaggtcattttactactcggtacatcacggaaaggaactacccctcccgccaggggcccagctaccagaacgatcgtgacttgatagagctggttgacccaaactggccagatctggggtctaaatggctatattctgtgccatggggatttaggaggctccttaactttgctcagactcaatacggtgatcctcccatatatgtgatggaaaatggagcatctcaaaaattccactgtactcaattatgtgatgagtggagaattcaataccttaaaggatacataaatgaaatgctaaaagctataaaagatggtgctaatataaaggggtatacttcctggtctctgttggataagtttgaatgggagaaaggatactcagatagatatggattctactatgttgaatttaacgacagaaataagcctcgctatccaaaggcttcagttcaatattacaagaagattatcattgccaatgggtttcccaatccaagagaggtggaaagttggtacctcaaagctttggaaacttgctctatcaacaatcagatgcttgctgcagagcctttgctaagtcacatgcaaatggttacggagatcgtggtacccactgtctgctccctctgtgtcctcatcactgctgttctactaatgctcctcctgaggaggcagagctgagacaggattatcaattttggagcttcataagagaatcttcaggatcttcctcccttttctgctttgagggtttccatacattgctgttttcaggttctacaataattacctttttttctctttctctttttggcttgtgcttatagaggacttcatcagcacacttcacttgaaatgcacctggctgcaggtaaccatgttagtaaagaatatcctttcttccaggctaccgtttagtgttaattccgaaaaatcaggtttgtgaaaattcagatggtctatgggtgagctgtcactcagttgccaaactgtcttaatcttcttttgaccactatatatttccactctccatttctgtggcttctcactttgatataagttctttacagcagttggtctgactggcagctgaaaaatgtgttgctc

SEQ ID NO: 19 - DNA, 1657 bp, Homo sapiens
  misc_feature: Incyte ID No 2706884CB1
ggcagttgtaaagtcgctggccagctagtggagtggagactgcagagggagataaagagagagggcaaagaggcagcaagagatttgtcctggggatccagaaacccatgataccctactgaacaccgaatcccctggaagcccacagagacagagacagcaagagaagcagagataaatacactcacgccaggagctcgctcgctctctctctctctctctcactcctccctccctctctctctgcctgtcctagtcctctagtcctcaaattcccagtcccctgcaccccttcctgggacactatgttgttctccgccctcctgctggaggtgatttggatcctggctgcagatgggggtcaacactggacgtatgagggcccacatggtcaggaccattggccagcctcttaccctgagtgtggaaacaatgcccagtcgcccatcgatattcagacagacagtgtgacatttgaccctgatttgcctgctctgcagccccacggatatgaccagcctggcaccgagcctttggacctgcacaacaatggccacacagtgcaactctctctgccctctaccctgtatctgggtggacttccccgaaaatatgtagctgcccagctccacctgcactggggtcagaaaggatccccaggtgggtcagaacaccagatcaacagtgaagccacatttgcagagctccacattgtacattatgactctgattcctatgacagcttgagtgagcttgctgagaggcctcagggcctggctgtcctgggcatcctaattgaggtgggtgagactaagaatatagcttatgaacacattctgagtcacttgcatgaagtcaggcataaagatcagaagacctcagtgcctcccttcaacctaagagagctgctccccaaacagctggggcagtacttccgctacaatggctcgctcacaactcccccttgctaccagagtgtgctctggacagttttttatagaaggtcccagatttcaatggaacagctggaaaagcttcaggggacattgttctccacagaagaggagccctctaagcttctggtacagaactaccgagcccttcagcctctcaatcagcgcatggtctttgcttctttcatccaaggatcctcgtataccacaggaagaagaggcttgaaaaccgaaagagtgtggtcttcacctcagcacaagccacgactgaggcataaattccttctcagataccatggatgtggatgacttcccttcatgcctatcaggaagcctctaaaatggggtgtaggatctggccagaaacactgtaggagtagtaagcagatgtcctccttcccctggacatctcctagagaggaatggacccaggctgtcattccaggaagaactgcagagccttcagcctctccaaacatgtaggaggaaatgaggaaatcgctgtgttgttaatgcagagaacaaactctgtttagttgcaggggaagtttgggatataccccaaagtcctctaccccctcacttttatggccctttccctagatatactgcgggatctctccttaggataaagagttgctgttgaagttgtatatttttgatcaatatatttggaaattaaagtttctgactttaaaaaaaaa

SEQ ID NO: 20 - DNA, 2331 bp, Homo sapiens
  misc_feature: Incyte ID No 4974616CB1
gcgcctgctgggcttgatctgaggctgaatcccgtgtgtggaccgccgttccctcttcgcaggatcgttggccaggatagcagatgcatttttgtagaagcattttgaggatgaatgtttgcaaatcattttgaaattgccaagtgccacatacatggaatgtatcctgctttcatcttggagttcacctgtgtggcttggatttatcacagtagcatttgtcttcaatctgtgtgttaactagaaatcaaggaaagacatgaggagatttgtctactgcaaggtggttctagccacttcgctgatgtgggttcttgttgatgtcttcttactgctgtacttcagtgaatgtaacaaatgtgatgacaagaaggagagatctctgctgcctgcattgagggctgttatttcaagaaaccaagaagggccaggagaaatgggaaaagctgtgttgattcctaaagatgaccaggagaaaatgaaagagctgtttaaaatcaatcagtttaaccttatggccagtgatttgattgcccttaatagaagtctgccagatgtaagattagaaggatgtaagacaaaagtctaccctgatgaacttccaaacacaagtgtagtcattgtgtttcataatgaagcttggagcactctccttagaactgtttacagtgtgataaatcgttccccacactatctactctcagaggtcatcttggtagatgatgccagtgaaagagattttctcaagttgacattagagaattacgtgaaaaatttagaagtgccagtaaaaattattaggatggaagaacgctctgggttaatacgtgcccgtcttcgaggagcagctgcttcaaaagggcaggtcataacttttcttgatgcacactgtgaatgcacgttaggatggctggagcctttgctggcaagaataaaggaagacaggaaaacggttgtctgccctatcattgatgtgattagtgatgatacttttgaatatatggctgggtcagacatgacttatgggggttttaactggaaactgaatttccgctggtatcctgttccccaaagagaaatggacaggaggaaaggagacagaacattacctgtcaggacccctactatggctggtggcctattttctattgacagaaactactttgaagagataggaacttacgatgcaggaatggatatctggggtggagagaatcttgaaatgtcttttaggatttggcaatgtggaggctccttggagattgttacttgctcccatgttggtcatgtttttcggaaggcaactccatacacttttcctggtggcactggtcatgtcatcaacaagaacaacaggagactggcagaagtttggatggatgaatttaaagatttcttctacatcatatccccaggtgttgtcaaagtggattatggagatgtgtcagtcagaaaaacactaagagaaaatctgaagtgtaagcccttttcttggtacctagaaaacatctatccggactcccagatcccaagacgttattactcacttggtgagataagaaatgttgaaaccaatcagtgtttagacaacatgggccgcaaggaaaatgaaaaagtgggtatattcaactgtcatggtatgggaggaaatcaggtattttcttacactgctgacaaagaaatccgaaccgatgacttgtgcttggatgtttctagactcaatggacctgtaatcatgttaaaatgccaccatatgagaggaaatcagttatgggaatatgatgctgagagactcacgttgcgacatgttaacagtaaccaatgtctcgatgaaccttctgaagaagacaaaatggtgcctacaatgcaggactgtagtggaagcagatcccaacagtggctgctaaggaacatgaccttgggcacatgaagatcatgtcctccaagccatgaaagtgtctacgcttttgtttttccattatttcaattgggggaaaatattaactttgctgaattgaaagttttaaaaatccttttagtattctaaaacacaattgtttctaattcgtttctagaaatgtttgcttatttccctactaaaatttgtatctgatcaaagcacataagaatataaataatagcaaactactattaaacaacagaacaacttgtaaaacaaattgtgtttgctttaagaaaaatctttattgcactcatgtcatagggttaattggaggttattttatttttggttgtcatggtgattgaaagagataatgtaaatgccttataaaatcttcattatgaaatattatcagttgctttataaac

SEQ ID NO: 21 - DNA, 3439 bp, Homo sapiens
  misc_feature: Incyte ID No 70861047CB1
gcgattccgttctccccaccaccaatccgacctcccagccgtctccgccgccgagcatacttgaggtgggacgagcaggggcttggatccctgccggccgtctggtgtgtgaggcttgcacggcccctggctgccccgcgcctcgccggagcccgagggggcgcaggtccggggcgagggccggccgggctgtttgatggcttcactgagaagagtcaaagtgctgttggtgttgaacttgatcgcggtagccggcttcgtgctcttcctggccaagtgccggcccatcgcggtgcgcagcggagacgccttccacgagatccggccgcgcgccgaggtggccaacctcagcgcgcacagcgccagccccatccaggatgcggtcctgaagcgcctgtcgctgctggaggacatcgtgtaccggcagctgaatggcttatccaaatcccttgggctcattgaaggttatggtgggcggggtaaagggggccttccggctactctttccccggctgaagaagaaaaggctaagggaccccatgagaagtatggctacaattcatacctcagtgaaaaaatttcactggaccgttccattccggattatcgtcccaccaagtgtaaggagctcaagtactccaaggacctgccccagatatccatcatattcatcttcgtgaacgaggccctgtcggtgatcctgcggtccgtgcacagtgccgtcaatcacacgcccacacacctgctgaaggaaatcattctggtggatgacaacagcgacgaagaggagctgaaggtccccctagaggagtatgtccacaaacgctaccccgggctggtgaaggtggtaagaaatcagaagagggaaggcctgatccgcgctcgcattgagggctggaaggtggctaccgggcaggtcactggcttctttgatgcccacgtggaattcaccgctggctgggctgagccggttctatcccgcatccaggaaaaccggaagcgtgtgatcctcccctccattgacaacatcaaacaggacaactttgaggtgcagcggtacgagaactcggcccacgggtacagctgggagctgtggtgcatgtacatcagccccccaaaagactggtgggacgccggagacccttctctccccatcaggaccccagccatgataggctgctcgttcgtggtcaacaggaagttcttcggtgaaattggtcttctggatcctggcatggatgtatacggaggagaaaatattgaactgggaatcaaggtatggctctgtgggggcagcatggaggtccttccttgctcacgggtggcccacattgagcggaagaagaagccatataatagcaacattggcttctacaccaagaggaatgctcttcgcgttgctgaggtctggatggacgattacaagtctcatgtgtacatagcgtggaacctgccgctggagaatccgggaattgacatcggtgatgtctccgaaagaagagcattaaggaaaagtttaaagtgtaagaatttccagtggtacctggaccatgtttacccagaaatgagaagatacaataataccgttgcttacggggagcttcgcaacaacaaggcaaaagacgtctgcttggaccaggggccgctggagaaccacacagcaatattgtatccgtgccatggctggggaccacagcttgcccgctacaccaaggaaggcttcctgcacttgggtgccctggggaccaccacactcctccctgacacccgctgcctggtggacaactccaagagtcggctgccccagctcctggactgcgacaaggtcaagagcagcctgtacaagcgctggaacttcatccagaatggagccatcatgaacaagggcacgggacgctgcctggaggtggagaaccggggcctggctggcatcgacctcatcctccgcagctgcacaggtcagaggtggaccattaagaactccatcaagtagagggagggagctggggcactggagcctggcccccaggacatggctgctccccccaacatctggaccagctgccctggcggagagacagcaaggggccggcaggtgctcgatgggccccccagggcttctccagggcagcacagggaccccggatgaagactctgtcccccctcaggcattcagctgcccacaagtttcctgcaccctggaaaagccccccacccttcctctgggaaactgacagctgtcttccacagcctctgatgtggacctggtactgaggagcaagactgtccagttctcctccacatctcccatcccagaatcaggatctgggactggcagggtcccctcctgtgtctcatctcttgcagcagcagctgctgaactccagccatcaacacggtgggaggcagcgggggcttcagccatgtcctagctccccgccctaaaaggaggcagtgaggaccaggcactatttcctccgaggttacttctacccagatgacacctgcctgttcacgccccaaggcagctactgcccctaacccttcccaccagggtagctttgggcactgcagctctggacttttctggcccctcctgagatgacctgatggagctgatgctttctctcctaatccctgggcactaggctcttatcagtgtgcttgggccagctctcctgcctgtgtctagaggaagccagagacagaaataggctaagcctgcagtaggatctcagccacaagggccccgcaggatggagctgggtcaaggaccagggagccctgactcccagaggctgccaccggggagaagcagcggtcctccatccagaacctaagggctgaagcaaaggctgccaggacccttgaagatgcttttggctcacctcatttcaccccacgctctgctggctggcagaggagaaggcagtcgtttcctctctgaagagtatttttttcgattgccctctggttagggtgcacatataaatcagagttaatatatgaacgcgtgtgcatgcacaagtgtgtgtgtgcctgcgtgctgtgcgtggcagggtgtgtgtgtgtgtgtctggctgtgcgttccggagtgtgtgacgatgctgacctagctgtgtggccttgggcttgctgcttcattactcacctggatggggacgagggatgagaagggtgtgggtttggccccatgtcactggccggaaggatgtgtctcagccctgccctgtggggtgcccccgatgggaggctgtcccatctcccagtccccatctctttttccccacactgtccctggccaagccctgcccagagctgaaccctgtagctgcccccttgccctgtgtgggattcgcagtgtctcatttggtgacgtcttactggtgatcatctcctcaccccatctcccaccttgtggaataaatacatgttagcc

SEQ ID NO: 22 - DNA, 2749 bp, Homo sapiens
  misc_feature: Incyte ID No 7472794CB1
cacgagcgagcaagtgccgaaagttaatgccccgggaatgttcaatatagtggttcttacattttagtgtttatcagaatcacccagagggcaggttgcaacacacatcactaggcctctccttctacgaggtagggcccaaaatttgcatttctaacagcttcccactgcttatttgccttggatgaatgacaatatgggcattttgatgctataaacaaatgctgtcaccatagaactagactttacctataacctatttcagcccccttatttatagtctactttcccatataaaactaagatttatatataggggtgtttgggggtatgcaaatgaatatataacatacatgcatacacatatatatacattcccttcatttcttttatatgtataggtatatactcatagaattttgataagataataaattttaaccctttgattacatatgaaaaatttgaggaccagagaaaataaatgactttttcaagattatattctttataatcagtactggaggcaaagccagaatgctgccattttaattccaatctgttattttcactaaatcatgtatccttttttataatgaaaattaaaagggctactggactgatacacagctgaaaaccctcagttctggactgaactcccagcagtttctaattaggtgtggagttgcaagagctctggaaaagatgttggtgctaaactgttctaccaaattactgatactggagaaaatgttgaagagttgctttcctgaatctctcaaggtttacggagcggtgatgaacataaatcgtgggaacccctttcaaaaggaagtggtgttggattcatggccggatttcaaagctgttatcacccgacgacaaagagaggctgagacagataaccttgatcattatactaatgcctatgctgtgttctacaaggatgtcagggcttatcgacagctattggaagaatgtgatgtttttaactgggaccaagtttttcaaatacaaaaggggccttccccacgactaacctacctgagtgttgccaatgcggatctactcaaccggacttggtcccggggaggcaatgaacaatgtctccggtacatcgccaacctcatctcctgcttccctagtgtgtgtgtccgggatgagaagggaaacccggtctcctggtccatcacagaccagtttgccaccatgtgccatggctacaccctgccagaacatcgcaggaaaggttacagccggctggtggccctcacgctggccaggaagttgcaaagccggggattcccctctcaggggaacgtcctggatgacaacacggcgtctataagcctcctgaagagtctccatgctgagttcttgccttgtcgcttccacaggcttattctcacccctgcgactttctctggcctgcctcacctctagcccagtaaaaaactgcagtggttttattactttccctgagcatacacacactcttggctgccaacgaggggagagttaaaatgggaatcaggggactcttgagttgttggaaagggtctggagaatatatacaggatccacttgagaagccttaatttttcgtatctcaggtttctccagtaaatagctgtgggggtgaagagtagctgtggctgaagactgaggacgattgtcctcctgtaggatccactgtaggagaataggttctaaagccagcagttttagtgtactaggagaaattactgcatgagaacaaatgatttaacagaggaccacgtggctactgctttttgattgctgcttggacctctgctctgtattcttaaagccacaccgcttccctactgccatcatattcccctgtccccactgctatgtctcatcaacctctgttcctaacacctctgccaccaagttctctgtagagtaacctcctttttcccctttaattacttgctctttacttctgcctaggactctagcctatagttcactgccctgggaatgttcaaatatagtggttcttacattttagtgtttatcagaatcacccagagggcaggttgcaacacacatcactaggcctctccttctacgaggtagggcccaaaatttgcatttctaacagcttcccactgcttatttgccttggatgaatgacaatatgggcattttgatgctataaacaaatgctgtcaccatagaactagactttacctataacctatttcagcccccttatttatagtctactttcccatataaaactaagatttatatataggggtgtttgggggtatgcaaatgaatatataacatatatgcatacacatatatatacattctcttcatttcttttatatgtataggtatatactcatagaattttgataagataataaattttaaccctttgattacatatgaaaaatttgaggaccagagaaaataaatgactttttcaagattatattctttataatcagtactggaggcaaagccagaatgctgccattttaattccaatctgttattttcactaaatcatgtatccttttttataatgaaaattaaaagggctactggactgatacacagctgaaaaccctcagttctggactgaactcccagcagtttctaattaggtgtggagttgcaagagctctggaaaagatgttggtgctaaactgttctaccaaattactgatactggggaaaatgttgaagagttgctttcctgaatctc

(19) United States
(12) Patent Application Publication
(10) Pub. No.: US 2004/0265807 A1
(43) Pub. Date: Dec. 30, 2004

(54) Enzymes

(76) Inventors: Madhusudan M Sanjanwala, Los Altos, CA (US); Yan Lu, Mountain View, CA (US); Ernestine A Lee, Castro Valley, CA (US); April J A Hafalia, Daly City, CA (US); Bridget A Warren, San Marcos, CA (US); Mariah R Baughn, Los Angeles, CA (US); Y Tom Tang, San Jose, CA (US); Henry Yue, Sunnyvale, CA (US); Monique G Yao, Mountain View, CA (US); Sally Lee, San Jose, CA (US); Michael B Thornton, Oakland, CA (US); Narinder K Chawla, Union City, CA (US); Yuming Xu, Mountain View, CA (US); Uyen K Tran, San Jose, CA (US); Preeti G Lal, Santa Clara, CA (US); Dyung Aina M Lu, San Jose, CA (US); Anita Swarnakar, San Francisco, CA (US); Huijun Z Ring, Foster City, CA (US); Karen A Jones, Bollington (GB)

Correspondence Address: FOLEY AND LARDNER, SUITE 500, 3000 K STREET NW, WASHINGTON, DC, 20007, US

(21) Appl. No.: 10/467,903

(22) PCT Filed: Feb. 8, 2002
(86) PCT No.: PCT/US02/03814

Related U.S. Application Data
(60) Provisional application No. 60/268,113, filed on Feb. 9, 2001.
(60) Provisional application No. 60/269,215, filed on Feb. 15, 2001.
(60) Provisional application No. 60/272,271, filed on Feb. 27, 2001.
(60) Provisional application No. 60/274,091, filed on Mar. 7, 2001.
(60) Provisional application No. 60/274,423, filed on Mar. 9, 2001.
(60) Provisional application No. 60/278,480, filed on Mar. 23, 2001.
(60) Provisional application No. 60/278,479, filed on Mar. 23, 2001.

(51) Int. Cl. C12Q 1/68; C07H 21/04; C12N 9/00

(52) U.S. Cl. 435/6; 435/69.1; 435/183; 435/320.1; 435/325; 536/23.2

(57) ABSTRACT

The invention provides human enzymes (NZMS) and polynucleotides which identify and encode NZMS. The invention also provides expression vectors, host cells, antibodies, agonists, and antagonists. The invention also provides methods for diagnosing, treating, or preventing disorders associated with aberrant expression of NZMS.

### TECHNICAL FIELD

This invention relates to nucleic acid and amino acid sequences of enzymes and to the use of these sequences in the diagnosis, treatment, and prevention of immune system disorders, immune deficiencies, developmental disorders, eye disorders, metabolic disorders, smooth muscle disorders, neurological disorders, pulmonary disorders, parasitic infections, and cell proliferative disorders including cancer, and in the assessment of the effects of exogenous compounds on the expression of nucleic acid and amino acid sequences of enzymes.

### BACKGROUND OF THE INVENTION

Oxidoreductases

Eukaryotic cells extract energy and synthesize macromolecules by a complex series of oxidation-reduction reactions collectively referred to as aerobic metabolism. One consequence of aerobic metabolism is the production of free radicals in the form of superoxides (O₂.—) and hydroxyl ions (OH.). Superoxides are produced within cells by mitochondria and the endoplasmic reticulum as a consequence of “leakage” of electrons onto O₂ from their correct paths in electron transfer chains. Hydroxyl ions are produced by ionizing radiation and by the reaction of O₂.— with hydrogen peroxide (H₂O₂) at iron- or copper-containing sites. Free radicals, especially hydroxyl ions, are extremely reactive and can interact with almost all molecules, including proteins, carbohydrates, DNA, and lipids. These interactions can lead to the formation of nonradical hydroperoxides, such as phospholipid hydroperoxides. Interaction of hydroxyl ions with DNA may be a significant contributor to the age-dependent development of cancer. Cells also use free radicals and their derivatives in beneficial ways, such as cytochrome P45O-mediated oxidations, regulation of smooth muscle tone, and killing of microorganisms by macrophages and granulocytes (Bast, A. et al. (1991) Am. J. Med. 91(3C):2S-13S).

Defects in enzymes involved in oxidation and reduction reactions in cells (oxidoreductases) lead to imbalances in the oxidation potential within cells, frequently with clinical manifestations. For example, an excess of superoxide dismutase (SOD), an enzyme that detoxifies superoxide compounds, may be relevant to the clinical condition known as Down's Syndrome. Low antioxidant levels or high O₂.— and H₂O₂ levels produce oxidative stress. Oxidative stress induced by phagocytes at sites of chronic inflammation lead to rheumatoid arthritis in the joints and inflammatory bowel diseases in the intestine. Asthma is also a manifestation of an inflammatory reaction in the lung and is related to oxygen free radical formation (Sies, H. (1991) Am. J. Med. 91 (3C):31S-38S).

Proteins involved in oxidation and reduction also have specific functions in synthesis, catalysis, salvage, and detoxification within cells. Defects in these enzymes are likely to lead to the accumulation of toxic precursor molecules within cells or the failure to synthesize compounds critical for cell viability (see examples, below). In addition to their activities on naturally occurring substrates, oxidoreductases are also closely associated with drug metabolism and pharmacokinetics. Inherited differences in drug metabolism lead to drastically different levels of drug efficacy and toxicity among individuals. For drugs with narrow therapeutic indices, or drugs which require bioactivation (such as codeine), these polymorphisms can be critical. Moreover, promising new drugs are frequently eliminated in clinical trials based on toxicities which may only affect a segment of the patient group. Advances in pharmacogenomics research, of which drug metabolizing enzymes constitute an important part, are promising to expand the tools and information that can be brought to bear on questions of drug efficacy and toxicity (Evans, W. E. and R. V. Relling (1999) Science 286:487-491).

The properties of selected oxidoreductases (i.e., glutathione peroxidases, glutathione S-transferase, glutaredoxin, peroxisomal β-oxidation enzymes, protein disulfide isomerases, thioredoxins, aldo/keto reductases, aldehyde dehydrogenases, alcohol dehydrogenases, acyl-CoA dehydrogenase, 6-phosphogluconate dehydrogenase, ribonucleotide diphosphate reductase, dihydrodiol dehydrogenase, 15-oxoprostaglandin 13-reductase, 15-hydroxyprostaglandin dehydrogenase, glucose-methanol-choline oxidoreductases, and other secreted redox proteins) associated with acquired and inherited genetic diseases and drug metabolism, are described, below.

Glutathione Peroxidases

The family of glutathione peroxidases encompass three tetrameric glutathione peroxidases (GPx1-3) and the monomeric phospholipid hydroperoxide glutathione peroxidase (PHGPx/GPx4). Although the overall homology between tetrameric enzymes and GPx4 is less than 30% o, a pronounced similarity has been detected in clusters involved in the active site and a common catalytic triad has been defined by structural and kinetic data (Epp, O. et al. (1983) Eur. J. Biochem. 133:51-69). The family members show different tissue distributions. GPx1 is ubiquitously expressed in cells, whereas GPx2 is present in the liver and colon, and GPx3 is present in plasma. GPx4 is found at low levels in all tissues but is expressed at high level in the testis. These tissue localization patterns may be important for regulating the level and targets of glutathione peroxidase activity (Ursini, F. et al (1995) Meth. Enzymol. 252:38-53).

GPx4 is unique in both its structure and activity. GPx4 is the only monomeric glutathione peroxidase found in mammals. It is also the only mammalian glutathione peroxidase to show high affinity for and reactivity with phospholipid hydroperoxides, and to be membrane associated. The inhibition of lipid peroxidation by GPx4 requires glutathione and physiological levels of vitamin E, suggesting a tandem mechanism for the antioxidant activities of GPx4 and vitamin E. GPx4 also has alternative transcription and translation start sites which determine its subcellular localization (Esworthy, R. S. et al. (1994) Gene 144:317-318; and Maiorino, M. et al. (1990) Meth. Enzymol. 186:448-450).

Glutathione S-Transferases (GST)

The glutathione S-transferases (GST) are a ubiquitous family of enzymes with dual substrate specificities that perform important biochemical functions of xenobiotic biotransformation and detoxification, drug metabolism, and protection of tissues against peroxidative damage. The basic reaction catalyzed by these enzymes is the conjugation of an electrophile with reduced glutathione (GSH) and results in either activation or deactivation/detoxification of the chemical. The absolute requirement for binding reduced GSH to a wide variety of chemicals necessitates a diversity in GST structures in various organisms and cell types.

GSTs are homodimeric or heterodimeric proteins localized in the cell cytosol. The major isozymes share common structural and catalytic properties and, many have been classified into four major classes, Alpha, Mu, Pi, and Theta. The two largest classes, Alpha and Mu, are identified by their respective isoelectric points; pI˜7.5-9.0 (Alpha), and pI˜6.6 (Mu). Each GST possesses a common binding site for GSH and a variable hydrophobic binding site. The hydrophobic binding site in each isozyme is specific for particular electrophilic substrates. Specific amino acid residues within GSTs have been identified as important for these binding sites and for catalytic activity. Residues Q67, T68, D101, E104, and R131 are important for the binding of GSH (Lee, H.-C. et al. (1995) J. Biol. Chem. 270:99-109). Residues R13, R20, and R69 are important for the catalytic activity of GST (Stenberg, G. et al. (1991) Biochem. J. 274:549-555).

While GSTs normally perform the essential function of deactivation and detoxification of potentially mutagenic and carcinogenic chemicals, dysfunction or inappropriate expression of GSTs are detrimental. Some forms of rat and human GSTs are reliable preneoplastic markers of carcinogenesis. Expression of human GSTs in bacterial strains, such as Salmonella typhimurium, used in the well known Ames test for mutagenicity, has helped to establish the role of these enzymes in mutagenesis. Dihalomethanes, which produce liver tumors in mice, are believed to be activated by GST. This view is supported by the finding that dihalomethanes are more mutagenic in transformed bacterial cells expressing human GST than in non-transformed cells (Thier, R. et al. (1993) Proc. Natl. Acad. Sci. USA 90:8567-8580). The mutagenicity of ethylene dibromide and ethylene dichloride is increased in bacterial cells expressing the human Alpha GST, A1-1, while the mutagenicity of aflatoxin B1 is substantially reduced by enhancing the expression of GST (Simula, T. P. et al. (1993) Carcinogenesis 14:1371-1376). Thus, control of GST activity may be useful in the control of mutagenesis and carcinogenesis.

GST has been implicated in the acquired resistance of many cancers to drug treatment, the phenomenon known as multi-drug resistance (MDR). MDR occurs when a cancer patient is treated with a cytotoxic drug such as cyclophosphamide and subsequently becomes resistant to this drug and to a variety of other cytotoxic agents as well. Increased GST levels are associated with some of these drug resistant cancers, and it is believed that this increase occurs in response to the drug agent which is then deactivated by the GST catalyzed GSH conjugation reaction. The increased GST levels then protect the cancer cells from other cytotoxic agents for which GST has affinity increased levels of A1-1 in tumors has been linked to drug resistance induced by cyclophosphamide treatment (Dirven, H. A. et al. (1994) Cancer Res. 54:6215-6220). Thus control of GST activity in cancerous tissues may be useful in treating MDR in cancer patients.

Glutaredoxin

The reduction of ribonucleotides to the corresponding deoxyribonucleotides, needed for DNA synthesis during cell proliferation, is catalyzed by the enzyme ribonucleotide diphosphate reductase. Glutaredoxin is a glutathione (GSH)-dependent hydrogen donor for ribonucleotide diphosphate reductase and contains the active site consensus sequence C-P-Y-C-. This sequence is conserved in glutaredoxins from such different organisms as E. coli, vaccinia virus, yeast, plants, and mammalian cells. Glutaredoxin has inherent GSH-disulfide oxidoreductase (thioltransferase) activity in a coupled system with GSH, NADPH, and GSH-reductase, catalyzing the reduction of low molecular weight disulfides as well as proteins. Glutaredoxin has been proposed to exert a general thiol redox control of protein activity by acting both as an effective protein disulfide reductase, similar to thioredoxin, and as a specific GSH-mixed disulfide reductase (Padilla, C. A. et al. (1996) FEBS Lett. 378:69-73).

In addition to their important role in DNA synthesis and cell division, glutaredoxin and other thioproteins provide effective antioxidant defense against oxygen radicals and hydrogen peroxide (Schallreuter, K. U. and J. M. Wood (1991) Melanoma Res. 1:159-167). Glutaredoxin is the principal agent responsible for protein dethiolation in vivo and reduces dehydroascorbic acid in normal human neutrophils (Jung, C. H. and J. A. Thomas (1996) Arch. Biochem. Biophys. 335:61-72; Park, J. B. and M. Levine (1996) Biochem. J. 315:931-938).

Secreted Redox Proteins

Redox polypeptides are also released into the extracellular environment and may have similar or distinct functions compared to their intracellular homologues. Several cytokines or secreted cytokine-like factors such as adult T-cell leukemia-derived factor, 3B6-interleukin-1, T-hybridoma-derived (MP-6) B cell stimulatory factor, and early pregnancy factor have been reported to be identical to thioredoxin (Holmgren, A. (1985) Annu. Rev. Biochem. 54:237-271; Abate, C. et al. (1990) Science 249:1157-1161; Tagaya, Y. et al. (1989) EMBO J. 8:757-764; Wakasugi, H. (1987) Proc. Natl. Acad. Sci. USA 84:804-808; Rosen, A. et al. (1995) Int. Immunol. 7:625-633). Thus thioredoxin secreted by stimulated lymphocytes (Yodoi, J. and T. Tursz (1991) Adv. Cancer Res. 57:381-411; Tagaya, N. et al. (1990) Proc. Natl. Acad. Sci. USA 87:8282-8286) has extracellular activities including a role as a regulator of cell growth and a mediator in the immune system (Miranda-Vizuete, A. et al. (1996) J. Biol. Chem. 271:19099-19103; Yamauchi, A. et al. (1992) Mol. Immunol. 29:263-270).

The selenoprotein thioredoxin reductase is secreted by both normal and neoplastic cells and has been implicated as both a growth factor and as a polypeptide involved in apoptosis (Soderberg, A. et al. (2000) Cancer Res. 60:2281-2289). An extracellular plasmin reductase secreted by hamster ovary cells (T-1080) has been show to participate in the generation of angiostatin from plasmin. In this case, the reduction of the plasmin disulfide bonds triggers the proteolytic cleavage of plasmin which yields the angiogenesis inhibitor, angiostatin (Stathakis, P. et al (1997) J. Biol. Chem. 272:20641-20645). Low levels of reduced sulfhydryl groups in plasma has been associated with rheumatoid arthritis. The failure of these sulfhydryl groups to scavenge active oxygen species (e.g., hydrogen peroxide produced by activated neutrophils) results in oxidative damage to surrounding tissues and the resulting inflammation (Hall, N. D. et al. (1994) Rheumatol. Int. 4:35-38).

Protein Disulfide Isomerases, Thioredoxins, and Glutaredoxins

Cells contain a number of specialized molecules that assist in the formation of protein secondary and tertiary structure by orchestrating the formation of disulfide bonds. Although incubation of reduced, unfolded proteins in buffers with defined ratios of oxidized and reduced thiols can lead to native conformation, the rate of folding is slow and the attainment of native conformation decreases proportionately to the size and number of cysteines in the protein. Certain cellular compartments such as the endoplasmic reticulum of eukaryotes and the periplasmic space of prokaryotes are maintained in a more oxidized state than the surrounding cytosol. Correct disulfide formation can occur in these compartments but at a rate that is insufficient for normal cell processes and not adequate for synthesizing secreted proteins. The protein disulfide isomerases, thioredoxins and glutaredoxins are able to catalyze the formation of disulfide bonds and regulate the redox environment in cells to enable the necessary thiol:disulfide exchanges (Loferer, H. (1995) J. Biol. Chem. 270:26178-26183).

Each of these proteins have somewhat different functions but all belong to a group of disulfide-containing redox proteins that contain a conserved active-site sequence and are ubiquitously distributed in eukaryotes and prokaryotes. Protein disulfide isomerases are found in the endoplasmic reticulum of eukaryotes and in the periplasmic space of prokaryotes. They function by exchanging their own disulfide for a thiol in a folding peptide chain. In contrast, the reduced thioredoxins and glutaredoxins are generally found in the cytoplasm and function by directly reducing disulfides in the substrate proteins.

These catalytic molecules not only facilitate disulfide formation but also regulate and participate in a wide variety of physiological processes. The thioredoxin system serves, for example, as a hydrogen donor for ribonucleotide reductase and as a regulator of enzymes by redox control. It also modulates the activity of transcription factors such as NF-κB, AP-1, and steroid receptors.

Aldo/Keto Reductases

Aldo/keto reductases are monomeric NADPH-dependent oxidoreductases with broad substrate specificities (Bohren, K. M. et al. (1989) 3. Biol. Chem. 264:9547-9551). These enzymes catalyze the reduction of carbonyl-containing compounds, including carbonyl-containing sugars and aromatic compounds, to the corresponding alcohols. Therefore, a variety of carbonyl-containing drugs and xenobiotics are likely metabolized by enzymes of this class.

One known reaction catalyzed by a family member, aldose reductase, is the reduction of glucose to sorbitol, which is then further metabolized to fructose by sorbitol dehydrogenase. Under normal conditions, the reduction of glucose to sorbitol is a minor pathway. In hyperglycemic states, however, the accumulation of sorbitol is implicated in the development of diabetic complications (OMIM*103880 Aldo-keto reductase family 1, member B1). Members of this enzyme family are also highly expressed in some liver cancers (Cao, D. et al. (1998) J. Biol. Chem. 273:11429-11435).

Aldehyde Dehydrogenases

Aldehyde dehydrogenases catalyze the oxidation of aliphatic and aromatic aldehydes. The enzymes are present in most life forms. Representative enzymes include: (i) succinate-semialdehyde dehydrogenase, a NADP⁺-dependent enzyme in E. coli that reduces succinate semialdehyde to succinate, (ii) betaine-aldehyde dehydrogenase, an enzyme present in plants and bacteria that is involved in the biosythesis of betaine, a quaternary ammonium compound accumulated in response to dry conditions, (iii) delta-1-pyrroline-5-carboxylate dehydrogenase, an enzyme present in yeast that converts proline to glutamate, (iv) methylmalonate-semialdehyde dehydrogenase, an enzyme present in numerous species, from bacteria to mammals, that is involved in valine catabolism, and (v) formyltetrahydrofolate dehydrogenase, a cytosolic enzyme in mammals responsible for the NADP⁺-dependent decarboxylative reduction of 10-formyltetrahydrofolate to tetrahydrofolate and CO₂ as well as the NADP⁺-independent hydrolysis of 10-formyltetrahydrofolate to tetrahydrofolate and formate. The amino-terminal domain of rat liver 10-Formyltetrahydrofolate dehydrogenase (residues 1-203) is 24-30% identical to a group of glycinamide ribonucleotide transformylases (EC 2.1.2.1). The active site of these enzymes comprises a glutamic acid and a cysteine residue that are conserved in all enzymes of the family (Weretilnyk, E. A. and A. D. Hanson (1990) Proc. Natl. Acad. Sci. USA 87:2745-2749; Cook, R. J. et al. (1991) J. Biol. Chem. 266:4965-4973; Steele, M. I. et al. (1992) J. Biol. Chem. 267:13585-13592; and Krupenko, S. A. et al. (1995) 270:519-522).

Defects in members of the aldehyde dehydrogenase gene family have been linked directly to human diseases. For example, a defect in aldehyde dehydrogenase 10 (fatty aldehyde dehydrogenase) results in the autosomal recessive neurocutaneous disorder, Sjoegren-Larsson syndrome (SLS). This disease is characterized by severe mental retardation, spastic di or tetra-plegia and congenital ichthyosis (increased keratinization) which is usually evident at birth. Afflicted individuals may also present with white spots on the retina, seizures, short stature and speech defects (De Laurenzi, V. et al. (1996) Nat. Genet. 12:52-57). A defect in aldehyde dehydrogenase 4 (glutamate gamma-semialdehyde dehydrogenase; pyrroline-5-carboxylate dehydrogenase) results in hyperprolinemia, type II, an autosomal recessive disorder characterized by accumulation of plasma proline (10-15-fold excess). The clinical phenotype of this disorder varies from asymptomatic to neurological manifestations, including seizures and mental retardation (Hu, C. A. et al. (1996) J. Biol. Chem. 271:9795-9800).

In addition, the mitochondrial enzyme aldehyde dehydrogenase 2 catalyzes the second step in ethanol utilization:

Step 1: ethanol+NAD⁺→acetaldehyde+NADH (alcohol dehydrogenase)

Step 2: acetaldehyde+NAD⁺→acetic acid+NADH (aldehyde dehydrogenase)

Defects in aldehyde dehydrogenase result in acute alcohol intoxication. This genetic defect is very common in South-east Asians and South American Indians, while less common in Caucasians. The inactive variant allele encodes a single amino acid exchange (Hsu, L. C. et al. (1988) Genomics 2:57-65).

Alcohol Dehydrogenases

Alcohol dehydrogenases (ADHs) oxidize simple alcohols to the corresponding aldehydes. ADH is a cytosolic enzyme, prefers the cofactor NAD⁺, and also binds zinc ion. Liver contains the highest levels of ADH, with lower levels in kidney, lung, and the gastric mucosa.

Known ADH isoforms are dimeric proteins composed of 40 kDa subunits. There are five known gene loci which encode these subunits (a, b, g, p, c), and some of the loci have characterized allelic variants (b₁, b₂, b₃, g₁, g₂). The subunits can form homodimers and heterodimers; the subunit composition determines the specific properties of the active enzyme. The holoenzymes have therefore been categorized as Class I (subunit compositions aa, ab, ag, bg, gg), Class II (pp), and Class m (cc). Class I ADH isozymes oxidize ethanol and other small aliphatic alcohols, and are inhibited by pyrazole. Class II isozymes prefer longer chain aliphatic and aromatic alcohols, are unable to oxidize methanol, and are not inhibited by pyrazole. Class III isozymes prefer even longer chain aliphatic alcohols (five carbons and longer) and aromatic alcohols, and are not inhibited by pyrazole.

Peroxisomal β-Oxidation Enzymes

Another example of the importance of redox reactions in cell metabolism is the degradation of saturated and unsaturated fatty acids by mitochondrial and peroxisomal beta-oxidation enzymes which sequentially remove two-carbon units from Coenzyme A (CoA)-activated fatty acids. The main beta-oxidation pathway degrades both saturated and unsaturated fatty acids while the auxiliary pathway performs additional steps required for the degradation of unsaturated fatty acids.

The pathways of mitchondrial and peroxisomal beta-oxidation use similar enzymes, but have different substrate specificities and functions. Mitochondria oxidize short-, medium-, and long-chain fatty acids to produce energy for cells. Mitochondrial beta-oxidation is a major energy source for cardiac and skeletal muscle. In liver, it provides ketone bodies to the peripheral circulation when glucose levels are low as in starvation, endurance exercise, and diabetes (Eaton, S. et al. (1996) Biochem. J. 320:345-357). Peroxisomes oxidize medium-, long-, and very-long-chain fatty acids, dicarboxylic fatty acids, branched fatty acids, prostaglandins, xenobiotics, and bile acid intermediates. The chief roles of peroxisomal beta-oxidation are to shorten toxic lipophilic carboxylic acids to facilitate their excretion and to shorten very-long-chain fatty acids prior to mitochondrial beta-oxidation (Mannaerts, G. P. and PP. VanVeldhoven (1993) Biochimie 75:147-158).

The auxiliary beta-oxidation enzyme 2,4-dienoyl-CoA reductase catalyzes the following reaction:

trans-2, cis/trans-4-dienoyl-CoA+NADPH+H⁺--->trans-3-enoyl-CoA+NADP⁺

This reaction removes even-numbered double bonds from unsaturated fatty acids prior to their entry into the main beta-oxidation pathway (Koivuranta, K. T. et al. (1994) Biochem. J. 304:787-792). The enzyme may also remove odd-numbered double bonds from unsaturated fatty acids (Smeland, T. E. et al. (1992) Proc. Natl. Acad. Sci. USA 89:6673-6677).

Rat 2,4-dienoyl-CoA reductase is located in both mitochondria and peroxisomes (Dommes, V. et al. (1981) J. Biol. Chem. 256:8259-8262). Two immunologically different forms of rat mitochondrial enzyme exist with molecular masses of 60 kDa and 120 kDa (Hakkola, B. H. and J. K. Hiltunen (1993) Eur. J. Biochem. 215:199-204). The 120 kDa mitochondrial rat enzyme is synthesized as a 335 amino acid precursor with a 29 amino acid N-terminal leader peptide which is cleaved to form the mature enzyme (Hirose, A. et al. (1990) Biochim. Biophys. Acta 1049:346-349). A human mitochondrial enzyme 83% similar to rat enzyme is synthesized as a 335 amino acid residue precursor with a 19 amino acid N-terminal leader peptide (Koivuranta, supra). These cloned human and rat mitochondrial enzymes function as homotetramers (Koivuranta, supra). A Saccharomyces cerevisiae peroxisomal 2,4-dienoyl-CoA reductase is 295 amino acids long, contains a C-terminal peroxisomal targeting signal, and functions as a homodimer (Coe, J. G. S. et al. (1994) Mol. Gen. Genet. 244:661-672; and Gurvitz, A. et al. (1997) J. Biol. Chem. 272:22140-22147). All 2,4-dienoyl-CoA reductases have a fairly well conserved NADPH binding site motif of sequence (Koivuranta, supra).

The main pathway beta-oxidation enzyme enoyl-CoA hydratase catalyzes the following reaction: 2-trans-enoyl-CoA+H₂O<--->3-hydroxyacyl-CoA This reaction hydrates the double bond between C-2 and C-3 of 2-trans-enoyl-CoA, which is generated from saturated and unsaturated fatty acids (Engel, C. K. et al. (1996) EMBO J. 15:5135-5145). This step is downstream from the step catalyzed by 2,4-dienoyl-reductase. Different enoyl-CoA hydratases act on short-, medium-, and long-chain fatty acids (Eaton, supra). Mitochondrial and peroxisomal enoyl-CoA hydratases occur as both mono-functional enzymes and as part of multi-functional enzyme complexes. Human liver mitochondrial short-chain enoyl-CoA hydratase is synthesized as a 290 amino acid precursor with a 29 amino acid N-terminal leader peptide (Kanazawa, M. et al. (1993) Enzyme Protein 47:9-13; and Janssen, U. et at (1997) Genomics 40:470-475). Rat short-chain enoyl-CoA hydratase is 87% identical to the human sequence in the mature region of the protein and functions as a homohexamer (Kanazawa, supra; and Engel, supra). A mitochondrial trifunctional protein exists that has long-chain enoyl-CoA hydratase, 3-hydroxyacyl-CoA dehydrogenase, and long-chain 3-oxothiolase activities (Eaton, supra). In human peroxisomes, enoyl-CoA hydratase activity is found in both a 327 amino acid residue mono-functional enzyme and as part of a multi-functional enzyme, also known as bifunctional enzyme, which possesses enoyl-CoA hydratase, enoyl-CoA isomerase, and 3-hydroxyacyl-CoA hydrogenase activities (FitzPatrick, D. R. et al. (1995) Genomics 27:457-466; and Hoefler, G. et al. (1994) Genomics 19:60-67). A 339 amino acid residue human protein with short-chain enoyl-CoA hydratase activity also acts as an AU-specific RNA binding protein (Nakagawa, J. et al. (1995) Proc. Natl. Acad. Sci. USA 92:2051-2055). All enoyl-CoA hydratases share homology near two active site glutamic acid residues, with 17 amino acid residues highly conserved (Wu, W.-J. et al. (1997) Biochemistry 36:2211-2220).

Inherited deficiencies in mitochondrial and peroxisomal beta-oxidation enzymes are associated with severe diseases, some of which manifest themselves soon after birth and lead to death within a few years. Mitochondrial beta-oxidation associated deficiencies include, e.g., carnitine palmitoyl transferase and carnitine deficiency, very-long-chain acyl-CoA dehydrogenase deficiency, medium-chain acyl-CoA dehydrogenase deficiency, short-chain acyl-CoA dehydrogenase deficiency, electron transport flavoprotein and electron transport flavoproteinubiquinone oxidoreductase deficiency, trifrnctional protein deficiency, and short-chain 3-hydroxyacyl-CoA dehydrogenase deficiency (Eaton, supra). Mitochondrial trifunctional protein (including enoyl-CoA hydratase) deficient patients have reduced long-chain enoyl-CoA hydratase activities and suffer from non-ketotic hypoglycemia, sudden infant death syndrome, cardiomyopathy, hepatic dysfunction, and muscle weakness, and may die at an early age (Eaton, supra). A patient with a deficiency in mitochondrial 2,4-dienoyl-CoA reductase was hypotonic soon after birth, had feeding difficulties, and died at four months from respiratory acidosis (Roe, C. R. et al. (1990) J. Clin. Invest. 85:1703-1707).

Defects in mitochondrial beta-oxidation are associated with Reye's syndrome, a disease characterized by hepatic dysfunction and encephalopathy that sometimes follows viral infection in children. Reye's syndrome patients may have elevated serum levels of free fatty acids (Cotran, R. S. et al. (1994) Robbins Pathologic Basis of Disease, W. B. Saunders Co., Philadelphia Pa., p. 866). Patients with mitochondrial short-chain 3-hydroxyacyl-CoA dehydrogenase deficiency and medium-chain 3-hydroxyacyl-CoA dehydrogenase deficiency also exhibit Reye-like illnesses (Eaton, supra; and Egidio, R. J. et al. (1989) Am. Fam. Physician 39:221-226).

Inherited conditions associated with peroxisomal beta-oxidation include Zellweger syndrome, neonatal adrenoleukodystrophy, infantile Refsum's disease, acyl-CoA oxidase deficiency, peroxisomal thiolase deficiency, and bifunctional protein deficiency (Suzuki, Y. et al. (1994) Am. J. Hum. Genet 54:36-43; Hoefier, supra). Patients with peroxisomal bifunctional enzyme deficiency, including that of enoyl-CoA hydratase, suffer from hypotonia, seizures, psychomotor defects, and defective neuronal migration; accumulate very-long-chain fatty acids; and typically die within a few years of birth (Watkins, P. A. et al. (1989) J. Clin Invest. 83:771-777).

Peroxisomal beta-oxidation is impaired in cancerous tissue. Although neoplastic human breast epithelial cells have the same number of peroxisomes as do normal cells, fatty acyl-CoA oxidase activity is lower than in control tissue (e1 Bouhtoury, F. et al. (1992) J. Pathol. 166:27-35). Human colon carcinomas have fewer peroxisomes than normal colon tissue and have lower fatty-acyl-CoA oxidase and bifunctional enzyme (including enoyl-CoA hydratase) activities than normal tissue (Cable, S. et al. (1992) Virchows Arch. B Cell Pathol. Incl. Mol. Pathol. 62:221-226).

Acyl-CoA Dehydrogenases

The acyl-CoA dehydrogenase family comprises at least seven members of which four are involved in beta-oxidation of fatty acids (see above). Very long chain fatty acids, dicarboxylic fatty acids, some prostanoids, pristanic acid, bile acid intermediates, and xenobiotic compounds are degraded by beta-oxidation in mammalian peroxisomes (Van Veldhoven, P. P. et al. (1999) Adv. Exp. Med. Biol. 466:261-272). For example, very long chain acyl-CoA dehydrogenase (VLCAD), a homodimer of a 70-kDa mitochondrial membrane-associated protein (Souri, M. et al. (1998) FEBS Lett. 426:187-190), catalyzes die initial flavin-dependent oxidation of acyl-CoA fatty acids in the mitochondria. In the process, electrons are transferred to an electron-transferring flavoprotein. Patients with VLCAD deficiency present with early onset cardiomyopathy that results in a high incidence of hypoketotic hypoglycemia and infant death. These conditions may result from the failure to express VLCAD or the expression of mutated forms of VLCAD. In at least one case, a nucleotide change in the gene for VLCAD resulted in the incorrect splicing of the mRNA and the synthesis of a defective protein (Watanabe, H. et al. (2000) Hum. Mutat. 15:430-438). Mutations in exons 10 and 12 that result in amino acid substitutions or nonsense mutations have also been reported (He, G. et al. (1999) Biochem. Biophys. Res. Commun. 264:483-487). A lethal genetic illness has also been associated with a single amino acid substitution in a medium-chain acyl-CoA dehydrogenase (Yang, B. Z. (2000) Mol. Genet. Metab. 69:259-262).

The remaining three enzymes of the acyl-CoA dehydrogenase family are involved in the catabolism of amino acids (i.e., isovaleryl-CoA dehydrogenase, short/branched chain acyl-CoA dehydrogenase, and glutaryl-CoA dehydrogenase). Isovaleryl-CoA dehydrogenase (IVD), for example, catalyzes the conversion of isovaleryl-CoA to methylcrotonyl-CoA in the leucine catabolic pathway. IVD is a homotetramer of 175 kDa that contains one PAD prosthetic group per subunit. The subunits are synthesized with a 2 kDa N-terminal leader sequence that is proteolytically processed to yield the mature polypeptide. The gene encoding IVD maps to human chromosome 15 and spans 15 kilobases, consisting of 12 exons and 11 introns. Five different classes of mutations have been identified in cell lines from patients with isovaleric acidemia, a disease caused by a deficiency of IVD (Volchenboum, S. L. and J. Vockley (2000) J. Biol. Chem. 275:7958-7963 and Reinard, T. et al. (2000) J. Biol. Chem. 275:33738-33743).

6-Phosphogluconate Dehydrogenase

6-phosphogluconate dehydrogenase (6-PGDH) catalyses the NADP⁺-dependent oxidative decarboxylation of 6-phosphogluconate to ribulose 5-phosphate with the production of NADPH. The absence or inibition of 6-PGDH results in the accumulation of 6-phosphogluconate to toxic levels in eukaryotic cells. 6-PGDH is the third enzyme of the pentose phosphate pathway (PPP) and is ubiquitous in nature. In some heterofermentatative species, NAD+ is used as a cofactor with the subsequent production of NADH.

The reaction proceeds through a 3-keto intermediate which is decarboxylated to give the enol of ribulose 5-phosphate, then converted to the keto product following tautomerization of the enol (Berdis A. J. and P. F. Cook (1993) Biochemistry 32:2041-2046). 6-PGDH activity is regulated by the inhibitory effect of NADPH, and the activating effect of 6-phosphogluconate (Rippa, M. et al. (1998) Biochim. Biophys. Acta 1429:83-92). Deficiencies in 6-PGDH activity have been linked to chronic hemolytic anemia.

The targeting of specific forms of 6-PGDH (e.g., enzymes found in trypanosomes) has been suggested as a means for controlling parasitic infections (Tetaud, E. et al. (1999) Biochem. J. 338:55-60). For example, the T. brucei enzyme is markedly more sensitive to inhibition by the substrate analogue 6-phospho-2-deoxygluconate and the coenzyme analogue adenosine 2′,5′-bisphosphate, compared to the mammalian enzyme (Hanau, S. et al. (1996) Eur. J. Biochem. 240:592-599).

Ribonucleotide Diphosphate Reductase

Ribonucleotide diphosphate reductase catalyzes the reduction of ribonucleotide diphosphates (i.e., ADP, GDP, CDP, and UDP) to their corresponding deoxyribonucleotide diphosphates (i.e., dADP, dGDP, dCDP, and dUDP) which are used for the synthesis of DNA. Ribonucleotide diphosphate reductase thereby performs a crucial role in the de novo synthesis of deoxynucleotide precursors. Deoxynucleotides are also produced from deoxynucleosides by nucleoside kinases via the salvage pathway.

Mammalian ribonucleotide diphosphate reductase comprises two components, an effector-binding component (E) and a non-heme iron component (F). Component E binds the nucleoside triphosphate effectors while component F contains the iron radical necessary for catalysis. Molecular weight determinations of the E and F components, as well as the holoenzyme, vary according to the methods used in purification of the proteins and the particular laboratory. Component E is approximately 90-100 kDa, component F is approximately 100-120 kDa, and the holoenzyme is 200-250 kDa.

Ribonucleotide diphosphate reductase activity is adversely effected by iron chelators, such as thiosemicarbazones, as well as EDTA. Deoxyribonucleotide diphosphates also appear to be negative allosteric effectors of ribonucleotide diphosphate reductase. Nucleotide triphosphates (both ribo- and deoxyribo-) appear to stimulate the activity of the enzyme. 3-methyl-4-nitrophenol, a metabolite of widely used organophosphate pesticides, is a potent inhibitor of ribonucleotide diphosphate reductase in mammalian cells. Some evidence suggests that ribonucleotide diphosphate reductase activity in DNA virus (e.g., herpes virus)-infected cells and in cancer cells is less sensitive to regulation by allosteric regulators and a correlation exists between high ribonucleotide diphosphate reductase activity levels and high rates of cell proliferation (e.g., in hepatomas). This observation suggests that virus-encoded ribonucleotide diphosphate reductases, and those present in cancer cells, are capable of maintaining an increased supply deoxyribonucleotide pool for the production of virus genomes or for the increased DNA synthesis which characterizes cancers cells. Ribonucleotide diphosphate reductase is thus a target for therapeutic intervention cutter, L. M. and Y.-C. Cheng (1984) Pharmac. Ther. 26:191-207; and Wright, J. A. (1983) Pharmac. Ther. 22:81-102).

Dihydrodiol Dehydrogenase

Dihydrodiol dehydrogenases (DD) are monomeric, NAD(P)⁺-dependent, 34-37 kDa enzymes responsible for the detoxification trans-dihydrodiol and anti-diol epoxide metabolites of polycyclic aromatic hydrocarbons (PAH) such as benzo[a]yrene, benz[a]anthracene, 7-methyl-benz[alanthracene, 7,12-dimethyl-benz[a]anthracene, chrysene, and 5-methyl-chrysene. In mammalian cells, an environmental PAH toxin such as benzo[a]yrene is initially epoxidated by a microsomal cytochrome P450 to yield 7R,8R-arene-oxide and subsequently (−)-7R,8R-dihydrodiol ((−)-trans-7,8-dihydroxy-7,8-dihydrobenzo[a]pyrene or (−)-trans-B[a]P-diol) This latter compound is further transformed to the anti-diol epoxide of benzo[a]pyrene (i.e., (±)-anti-7β, 8-αdihydroxy-9α,10α-epoxy-7,8,9,10-tetrahydrobenzo[a]pyrene), by the same enzyme of a different enzyme, depending on the species. This resulting anti-diol epoxide of benzo[a]yrene, or the corresponding derivative from another PAH compound, is highly mutagenic.

DD efficiently oxidizes the precursor of the anti-diol epoxide (i.e., trans-dihydrodiol) to transient catechols which auto-oxidize to quinones, also producing hydrogen peroxide and semiquinone radicals. This reaction prevents the formation of the highly carcinogenic anti-diol. Anti-diols are not themselves substrates for DD yet the addition of DD to a sample comprising an anti-diol compound results in a significant decrease in the induced mutation rate observed in the Ames test. In this instance, DD is able to bind to and sequester the anti-diol, even though it is not oxidized. Whether through oxidation or sequestration, DD plays an important role in the detoxification of metabolites of xenobiotic polycyclic compounds (Penning, T. M. (1993) Chemico-Biological Interactions 89:1-34).

15-Oxoprostaglandin 13-Reductase

15-oxoprostaglandin 13-reductase (PGR) and 15-hydroxyprostaglandin dehydrogenase (15-PGDH) are enzymes present in the lung that are responsible for degrading circulating prostaglandins. Oxidative catabolism via passage through the pulmonary system is a common means of reducing the concentration of circulating prostaglandins. 15-PGDH oxidizes the 15-hydroxyl group of a variety of prostaglandins to produce the corresponding 15-oxo compounds. The 15-oxo derivatives usually have reduced biological activity compared to the 15-hydroxyl molecule. PGR further reduces the 13,14 double bond of the 15-oxo compound which typically leads to a further decrease in biological activity. PGR is a monomer with a molecular weight of approximately 36 kDa. The enzyme requires NADH or NADPH as a cofactor with a preference for NADH. The 15-oxo derivatives of prostaglandins PGE₁, PGE₂, and PGE_{2α}, are all substrates for PGR; however, the non-derivatized prostaglandins (i.e., PGE₁, PGE₂, and PGE_{2α}) are not substrates (Ensor, C. M. et al. (1998) Biochem. J. 330:103-108).

15-PGDH and PGR also catalyze the metabolism of lipoxin A₄ (LXA₄). Lipoxins (LX) are autacoids, lipids produced at the sites of localized inflammation, which down-regulate polymorphonuclear leukocyte (PMN) function and promote resolution of localized trauma. Lipoxin production is stimulated by the administration of aspirin in that cells displaying cyclooxygenase II (COX II) that has been acetylated by aspirin and cells that possess 5-lipoxygenase (5-LO) interact and produce lipoxin. 15-PGDH generates 15-oxo-LXA₄ with PGR further converting the 15-oxo compound to 13,14-dihydro-15-oxo-LXA₄ (Clish, C. B. et al. (2000) J. Biol. Chem. 275:25372-25380). This finding suggests a broad substrate specificity of the prostaglandin dehydrogenases and has implications for these enzymes in drug metabolism and as targets for therapeutic intervention to regulate inflammation.

GMC Oxidoreductases

The GMC (glucose-methanol-choline) oxidoreductase family of enzymes was defined based on sequence alignments of Drosophila melanogaster glucose dehydrogenase, Escherichia coli choline dehydrogenase, Aspergillus niger glucose oxidase, and Hansenula polymorpha methanol oxidase. Despite their different sources and substrate specificities, these four flavoproteins are homologous, being characterized by the presence of several distinctive sequence and structural features. Each molecule contains a canonical ADP-binding, beta-alpha-beta mononucleotide-binding motif close to the amino terminus. This fold comprises a four-stranded parallel beta-sheet sandwiched between a three-stranded antiparallel beta-sheet and alpha-helices. Nucleotides bind in similar positions relative to this chain fold (Cavener, D. R. (1992) J. Mol. Biol. 223:811-814; and Wierenga, R. K. et al. (1986) J. Mol. Biol. 187:101-107). Members of the GMC oxidoreductase family also share a consensus sequence near the central region of the polypeptide. Additional members of the GMC oxidoreductase family include cholesterol oxidases from Brevibacterium sterolicum and Streotomyces; and an alcohol dehydrogenase from Pseudomonas oleovorans (Cavener, D. R., supra; Henikoff, S. and J. G. Henikoff (1994) Genomics 19:97-107; van Beilen, J. B. et al. (1992) Mol. Microbiol. 6:3121-3136).

IMP Dehydrogenase/GMP Reductase

IMP dehydrogenase and GMP reductase are two oxidoreductases which share many regions of sequence similarity. EIP dehydrogenase (EC 1.1.1.205) catalyes the NAD-dependent reduction of IMP (inosine monophosphate) into XMP (xanthine monophosphate) as part of de novo GTP biosynthesis (Collart, P. R. and E. Huberman (1988) J. Biol. Chem. 263:15769-15772). GMP reductase catalyzes the NADPH-dependent reductive deamination of GMP into IMP, helping to maintain the intracellular balance of adenine and guanine nucleotides (Andrews, S. C. and J. R. Guest (1988) Biochem. J. 255:35-43).

Pyridine Nucleotide-Disulphide Oxidoreductases

Pyridine nucleotide-disulphide oxidoreductases are FAD flavoproteins involved in the transfer of reducing equivalents from FAD to a substrate. These flavoproteins contain a pair of redox-active cysteines contained within a consensus sequence which is characteristic of this protein family (Kurlyan, J. et al. (1991) Nature 352:172-174). Members of this family of oxidoreductases include glutathione reductase (EC 1.6.4.2); thioredoxin reductase of higher eukaryotes (EC 1.6.4.5); trypanothione reductase (EC 1.6.4.8); lipoamide dehydrogenase (EC 1.8.1.4), the B3 component of alpha-ketoacid dehydrogenase complexes; and mercuric reductase (EC 1.16.1.1).

Lactate Ferricytochrome C Oxidoreductase

The utilization of lactate requires two enzymes, the D and L-lactate ferricytochrome c oxidoreductase (D and L-LCR; EXPASY B. C. 1123 and E. C. 1124), which stereo-specifically oxidize D- and L-lactate to pyruvate (Lodi, T. et al. (1994) Mol. Gen. Genet. 244: 622-629). In yeast, these enzymes are nuclearly encoded and localized in mitochondria (Alberti A. et al. (2000) Yeast 16:657-665). D-LCR is linked to the respiratory chain with cytochrome C as the electron acceptor of the redox reaction. Both D- and L-LCR genes are controlled by the carbon source, being induced by the substrate lactate and repressed by glucose. (Lodi, T. et al. (1994) Mol. Gen. Genet. 244: 622-629).

Hydrolases

Hydrolases are a class of enzymes that catalyze the cleavage of various covalent bonds in a substrate by the introduction of a molecule of water. The reaction involves a nucleophilic attack by the water molecule's oxygen atom on a target bond in the substrate. The water molecule is split across the target bond, breaking the bond and generating two product molecules. Hydrolases participate in reactions essential to such functions as synthesis and degradation of cell components, and for regulation of cell functions including cell signaling, cell proliferation, inflamation, apoptosis, secretion and excretion. Hydrolases are involved in key steps in disease processes involving these functions. Hydrolytic enzymes, or hydrolases, may be grouped by substrate specificity into subclasses including phosphatases, peptidases, lysophospholipases, phosphodiesterases, glycosidases, glyoxalases, nibonucleases, thioether hydrolases, and hydrolases which act on carbon-nitrogen (C—N) bonds other than peptide bonds.

Phosphatases hydrolytically remove phosphate groups from proteins, an energy-providing step that regulates many cellular processes, including intracellular signaling pathways that in turn control cell growth and differentiation, cell-cell contact, the cell cycle, and oncogenesis.

Peptidases, also called proteases, cleave peptide bonds that form the backbone of peptide or protein chains. Proteolytic processing is essential to cell growth, differentiation, remodeling, and homeostasis as well as inflammation and the immune response. Since typical protein half-lives range from hours to a few days, peptidases are continually cleaving precursor proteins to their active form, removing signal sequences from targeted proteins, and degrading aged or defective proteins. Peptidases function in bacterial, parasitic, and viral invasion and replication within a host. Examples of peptidases include trypsin and chymotrypsin, components of the complement cascade and the blood-clotting cascade, lysosomal cathepsins, calpains, pepsin, renin, and chymosin (Beynon, R. J. and J. S. Bond (1994) Proteolytic Enzymes: A Practical Approach. Oxford University Press, New York, N.Y., pp. 1-5).

Lysophospholipases (LPLs) regulate intracellular lipids by catalyzing the hydrolysis of ester bonds to remove an acyl group, a key step in lipid degradation. Small LPL isoforms, approximately 15-30 kD, function as hydrolases; larger isoforms function both as hydrolases and transacylases. A particular substrate for LPLs, lysophosphatidylcholine, causes lysis of cell membranes. LPL activity is regulated by signaling molecules important in numerous pathways, including the inflammatory response.

The phosphodiesterases catalyze the hydrolysis of one of the two ester bonds in a phosphodiester compound. Phosphodiesterases are therefore crucial to a variety of cellular processes. Phosphodiesterases include DNA and RNA endo- and exo-nucleases, which are essential to cell growth and replication as well as protein synthesis. Endonuclease V (deoxyinosine 3′-endonuclease) is an example of a type II site-specific deoxyribonuclease, a putative DNA repair enzyme that cleaves DNAs containing hypoxanthine, uracil, or mismatched bases. Escherichia coli endonuclease V has been shown to cleave DNA containing deoxyxanthosine at the second phosphodiester bond 3′ to deoxyxanthosine, generating a 3′-hydroxyl and a 5′-phosphoryl group at the nick site (He, B. et al. (2000) Mutat. Res. 459:109-114). It has been suggested that Escherichia coli endonuclease V plays a role in the removal of deaminated guanine, i.e., xanthine, from DNA, thus helping to protect the cell against the mutagenic effects of nitrosative deamination (Schouten K A and Weiss B (1999) Mutat. Res. 435:245-254). In eukaryotes, the process of tRNA splicing requires the removal of small tRNA introns that interrupt the anticodon loop 1 base 3′ to the anticodon. This process requires the stepwise action of an endonuclease, a ligase, and a phosphotransferase (Hong, L. et al. (1998) Science 280:279-284). Ribonuclease P(RNase P) is a ubiquitous RNA processing endonuclease that is required for generating the mature tRNA 5′-end during the tRNA splicing process. This is accomplished through the catalysis of the cleavage of P-3′O bonds to produce 5′-phosphate and 3′-hydroxyl end groups at a specific site on pre-tRNA. Catalysis by RNase P is absolutely dependent on divalent cations such as Mg²⁺ or Mn²⁺ (Kurz, J. C. et al. (2000) Curr. Opin. Chem. Biol. 4:553-558). Substrate recognition mechanisms of RNase P have been demonstrated to be well conserved among the Eucarya, the Archaea, and the Bacteria (Fabbri, S. et al. (1998) Science 280:284-286). In S. cerevisiae, a gene designated POP1 for ‘processing of precursor RNAs’, encodes a protein component of both RNase P and RNase MRP, another RNA processing protein. Mutations in yeast POP1 have been shown to be lethal (Lygerou, Z. et al. (1994) Genes Dev. 8:1423-1433). Another phosphodiesterase is acid sphingomyelinase, which hydrolyzes the membrane phospholipid sphingomyelin to ceramide and phosphorylcholine. Phosphorylcholine is used in the synthesis of phosphatidylcholine, which is involved in numerous intracellular signaling pathways. Ceramide is an essential precursor for the generation of gangliosides, membrane lipids found in high concentration in neural tissue. Defective acid sphingomyelinase phosphodiesterase leads to a build-up of sphingomyelin molecules in lysosomes, resulting in Niemann-Pick disease.

Glycosidases catalyze the cleavage of hemiacetyl bonds of glycosides, which are compounds that contain one or more sugar. Mammalian lactase-phlorizin hydrolase, for example, is an intestinal enzyme that splits lactose. Mammalian beta-galactosidase removes the terminal galactose from gangliosides, glycoproteins, and glycosaminoglycans, and deficiency of this enzyme is associated with a gangliosidosis known as Morquio disease type B. Vertebrate lysosomal alpha-glucosidase, which hydrolyzes glycogen, maltose, and isomaltose, and vertebrate intestinal sucrase-isomaltase, which hydrolyzes sucrose, maltose, and isomaltose, are widely distributed members of this family with highly conserved sequences at their active sites.

The glyoxylase system is involved in gluconeogenesis, the production of glucose from storage compounds in the body. It consists of glyoxylase I, which catalyzes the formation of S-D-lactoylglutathione from methyglyoxal, a side product of triose-phosphate energy metabolism, and glyoxylase II, which hydrolyzes S-D-lactoylglutathione to D-lactic acid and reduced glutathione. Glyoxylases are involved in hyperglycemia, non-insulin-dependent diabetes mellitus, the detoxification of bacterial toxins, and in the control of cell proliferation and microtubule assembly.

Ribonucleases are enzymes which hydrolyze RNA and oligoribonucleotides. Ribonuclease T2 catalyzes the two-stage endonucleolytic cleavage of RNA to 3′-phosphomononucleotides and 3′-phosphooligonucleotides with 2′,3′-cyclic phosphate intermediates. Pancreatic ribonucleases (RNAse) (EC 3.1.27.5) are pyrimidine-specific endonucleases present in high quantity in the pancreas of a number of mammalian taxa and of a few reptiles. A number of other proteins belonging to the pancreatic RNAse family include kidney non-secretory ribonucleases (eosinophil-derived neurotoxin, EDN), liver-type ribonucleases, angiogenin, and eosinophil cationic protein (ECP) (PROSITE:PDOC00118). EDN is a distinct cationic protein of the eosinophil's large specific granule known primarily for its ability to induce ataxia, paralysis, and central nervous system cellular degeneration in experimental animals (Rosenberg, H. F. et al. (1989) PNAS 86:4460-4464).

A small subclass of hydrolases acting on ether bonds includes the thioether hydrolases. S-adenosyl-L-homocysteine hydrolase, also known as AdoHcyase or SAHH(PROSITE PDOC00603; EC 3.3.1.1), is a thioether hydrolase first described in rat liver extracts as the activity responsible for the reversible hydrolysis of S-adenosyl-L-homocysteine (AdoHcy) to adenosine and homocysteine (Sganga, M. W. et al. (1992) PNAS 89:6328-6332). SAHH is a cytosolic enzyme that has been found in all cells that have been tested, with the exception of Escherichia coli and certain related bacteria (Walker, R. D. et al. (1975) Can. J. Biochem. 53:312-319; Shimizu, S. et al. (1988) FEMS Microbiol. Lett. 51:177-180; Shimizu, S. et al. (1984) Eur. J. Biochem. 141:385-392). SAHH activity is dependent on NAD⁺ as a cofactor. Deficiency of SAHH is associated with hypermethioninemia (One Mendelian Inheritance in Man (OMIM) #180960 Hypermethioninemia), a pathologic condition characterized by neonatal cholestasis, failure to thrive, mental and motor retardation, facial dysmorphism with abnormal hair and teeth, and myocaridopathy (Labrune, P. et al. (1990) J. Pediat. 117:220-226).

Another subclass of hydrolases includes those enzymes which act on carbon-nitrogen (C—N) bonds other than peptide bonds. To this subclass belong those enzymes hydrolyzing amides, amidines, and other C—N bonds. This subclass is further subdivided on the basis of substrate specificity such as linear amides, cyclic amides, linear amidines, cyclic amidines, nitriles and other compounds.

A hydrolase belonging to the sub-subclass of enzymes acting on the cyclic amidines is adenosine deaminase (ADA). ADA catalyzes the breakdown of adenosine to inosine. ADA is present in many mammalian tissues, including placenta, muscle, lung, stomach, digestive diverticulum, spleen, erythrocytes, thymus, seminal plasma, thyroid, T-cells, bone marrow stem cells, and liver. A subclass of ADAs, ADAR, act on RNA and are classified as RNA editases. An ADAR from Drosophila, dADAR, has been shown to be expressed in the developing nervous system, making it a candidate for the editase that acts on para voltage-gated Na⁺ channel transcripts in the central nervous system (Palladino, M. J. et al. (2000) RNA 6:1004-1018). A deficiency of ADA causes profound lymphopenia with severe combined immunodeficiency (SCID). Cells from patients with ADA deficiency contain less than normal, and sometimes undetectable, amounts of ADA catalytic activity and ADA protein. It has been shown that ADA deficiency stems from genetic mutations in the ADA gene, resulting in SCID (Hershfield, M. S. (1998) Semin. Hematol. 4:291-298). Metabolic consequences of ADA deficiency in mice have been found to be associated with defects in alveogenesis, pulmonary inflammation, and airway obstruction (Blackburn, M. R. et al. (2000) J. Exp. Med. 192:159-170).

Pancreatic ribonucleases (RNase) are pyrimidine-specific endonucleases found in high quantity in the pancreas of certain mammalian taxa and of some reptiles (Beintema, J. J. et al (1988) Prog. Biophys. Mol. Biol. 51:165-192). Proteins in the mammalian pancreatic RNase superfamily are noncytosolic endonucleases that degrade RNA through a two-step transphosphorolytic-hydrolytic reaction (Beintema, J. J. et al. (1986) Mol. Biol. Evol. 3:262-275). Specifically, the enzymes are involved in endonucleolytic cleavage of 3′-phosphomononucleotides and 3′-phosphooligonucleotides ending in C—P or U—P with 2-3′-cyclic phosphate intermediates. Ribonucleases can unwind the DNA helix by complexing with single-stranded DNA; the complex arises by an extended multi-site cation-anion interaction between lysine and arginine residues of the enzyme and phosphate groups of the nucleotides. Some of the enzymes belonging to this family appear to play a purely digestive role, whereas others exhibit potent and unusual biological activities (D'Alessio, G. (1993) Trends Cell Biol. 3:106-109). Proteins belonging to the pancreatic RNase family include: bovine seminal vesicle and brain ribonucleases; kidney non-secretory ribonucleases (Beintema, J. J. et al (1986) FEBS Lett. 194:338-343); liver-type ribonucleases (Rosenberg, H. F. et al. (1989) PNAS U.S.A. 86:4460-4464); angiogenin, which induces vascularisation of normal and malignant tissues; eosinophil cationic protein (Hofsteenge, J. et al. (1989) Biochemistry 28:9806-9813), a cytotoxin and helminthotoxin with ribonuclease activity; and frog liver ribonuclease and frog sialic acid-binding lectin. The sequences of pancreatic RNases contain 4 conserved disulphide bonds and 3 amino acid residues involved in the catalytic activity.

A hydrolase belonging to the sub-subclass of enzymes acting only on asparagine-oligosaccharides containing one amino acid is N⁴-(β-N-acetylglucosaminyl)-L-asparaginase, or aspartylglucosylaminidase (AGA; EC 3.5.1.26. AGA is a key enzyme in the catabolism of N-linked oligosaccharides of glycoproteins. It cleaves the asparagine from the residual N-acetylglucosamines as one of the final steps in the lysosomal breakdown of glycoproteins. AGA is an enzyme of lysosomal origin that has been found in worms, rats, mice, pigs, humans, and flavobacteria (ExPASy Enzyme View of ENZYME: 3.5.1.2; SWISS-PROT P20933). A deficiency of AGA causes a lysosomal disease known as aspartylglucosaminuria (AGU) (Online Mendelian Inheritance in Man (OMIM) #208400 Aspartylglucosaminuria; Jenner, F. A. et al. (1967) Biochem. J. 103:48P-49P; Pollitt, R. J. et al. (1968) Lancet 11:253-255). Patients with AGU exhibit severe mental retardation, cranial asymmetry, scoliosis, periodic hyperactivity, and vacuolated lymphocytes. AGU in infants is characterized by diarrhea and frequent infections (Palo, J. et al. (1970) J. Ment. Defic. Res. 14:168-173). It has been shown that AGU stems from genetic mutations in the AGU gene, which probably affects the folding and stability of the AGA molecule (Ikonen, E. et al. (1991) PNAS 88:11222-11226; Ikonen, E. et al. (1991) EMBO J. 10:51-58; Ikonen, E. et al. (1991) Genomics 11:206-211). Metabolic consequences of AGA deficiency in mice have been found to be associated with defects in neuromotor coordination, including impaired bladder function and severe ataxic gait in older mice (Tenhunen, K. et al. (1995) Genomics 30:244-250; Gonzalez-Gomez, I. et al. (1998) Am. J. Path. 153:1293-1300).

Transferases

Transferases are enzymes that catalyze the transfer of molecular groups. The reaction may involve an oxidation, reduction, or cleavage of covalent bonds, and is often specific to a substrate or to particular sites on a type of substrate. Transferases participate in reactions essential to such functions as synthesis and degradation of cell components, regulation of cell functions including cell signaling, cell proliferation, inflammation, apoptosis, secretion and excretion. Transferases are involved in key steps in disease processes involving these functions. Transferases are frequently classified according to the type of group transferred. For example, methyl transferases transfer one-carbon methyl groups, amino transferases transfer nitrogenous amino groups, and similarly denominated enzymes transfer aldehyde or ketone, acyl, glycosyl, alkyl or aryl, isoprenyl, saccharyl, phosphorous-containing, sulfur-containing, or selenium-containing groups, as well as small enzymatic groups such as Coenzyme A.

Acyl transferases include peroxisomal carnitine octanoyl transferase, which is involved in the fatty acid beta-oxidation pathway, and mitochondrial carnitine palmitoyl transferases, involved in fatty acid metabolism and transport Choline O-acetyl transferase catalyzes the biosynthesis of the neurotransmitter acetylcholine. N-acyltransferase enzymes catalyze the transfer of an amino acid conjugate to an activated carboxylic group. Endogenous compounds and xenobiotics are activated by acyl-CoA synthetases in the cytosol, microsomes, and mitochondria. The acyl-CoA intermediates are then conjugated with an amino acid (typically glycine, glutamine, or taurine, but also ornithine, arginine, histidine, serine, aspartic acid, and several dipeptides) by N-acyltransferases in the cytosol or mitochondria to form a metabolite with an amide bond. One well-characterized enzyme of this class is the bile acid-CoA:amino acid N-acyltransferase (BAT) responsible for generating the bile acid conjugates which serve as detergents in the gastrointestinal tract (Falany, C. N. et al. (1994) J. Biol. Chem. 269:19375-9; Johnson, M. R. et al. (1991) J. Biol. Chem. 266:10227-33). BAT is also useful as a predictive indicator for prognosis of hepatocellular carcinoma patients after partial hepatectomy (Furutani, M. et al. (1996) Hepatology 24:1441-S).

N-acetyltransferases are cytosolic enzymes which utilize the cofactor acetyl-coenzyme A (acetyl-CoA) to transfer the acetyl group to aromatic amines and hydrazine containing compounds. In humans, there are two highly similar N-acetyltransferase enzymes, NAT1 and NAT2; mice appear to have a third form of the enzyme, NAT3. The human forms of N-acetyltransferase have independent regulation (NAT1 is widely-expressed, whereas NAT2 is in liver and gut only) and overlapping substrate preferences. Both enzymes appear to accept most substrates to some extent, but NAT1 does prefer some substrates (para-aminobenzoic acid, para-aminosalicylic acid, sulfamethoxazole, and sulfanilamide), while NAT2 prefers others (isoniazid, hydralazine, procainamide, dapsone, aminoglutethimide, and sulfamethazine). A recently isolated human gene, tubedown-1, is homologous to the yeast NAT-1 N-acetyltransferases and encodes a protein associated with acetyltransferase activity. The expression patterns of tubedown-1 suggest that it may be involved in regulating vascular and hematopoietic development (Gendron, R. L. et al. (2000) Dev. Dyn. 218:300-315).

Lysophosphatidic acid acyltransferase (LPAAT) catalyzes the acylation of lysophosphatidic acid (LPA) to phosphatidic acid. LPA is the simplest glycerophospholipid, consisting of a glycerol molecule, a phosphate group, and a mono-saturated fatty acyl chain. LPAAT adds a second fatty acyl chain to LPA, producing phosphatidic acid (PA). PA is the precursor molecule for diacylglycerols, which are necessary for the production of phospholipids, and for triacylglycerols, which are essential biological fuel molecules. In addition to being a crucial precursor molecule in biosynthetic reactions, LPA has recently been added to the list of intercellular lipid messenger molecules. LPA interacts with G protein-coupled receptors, coupling to various independent effector pathways including inilbition of adenylate cyclase, stimulation of phospholipse C, activation of MAP kinases, and activation of the small GTP-binding proteins Ras and Rho. (Moolenaar, W. H. (1995) J. Biol. Chem 28-:12949-12952.) The physiological effects of LPA have not been fully characterized yet, but they include promoting growth and invasion of tumor cells. PA, the product of LPAAT, is a key messenger in a common signaling pathway activated by proinflammatory mediators such as interleukin-1β, tumor necrosis factor α, platelet activating factor, and lipid A. (Bursten, S. L. et al. (1992) Am. J. Physiol. 262:C328-C338; Bursten S. L. et al. (1991) J. Biol. Chem. 255:20732-20743; Kester, M. (1993) J. Cell Physiol. 156:317-325.) Thus, LPAAT activity may mediate inflammatory responses to various proinflammatory agents.

Aminotransferases comprise a family of pyridoxal 5′-phosphate (PLP)-dependent enzymes that catalyze transformations of amino acids. Amino transferases play key roles in protein synthesis and degradation, and they contribute to other processes as well. For example, GABA aminotransferase (GABA-T) catalyzes the degradation of GABA, the major inhibitory amino acid neurotransmitter. The activity of GABA-T is correlated to neuropsycbiatric disorders such as alcoholism, epilepsy, and Alzheimer's disease (Sherif, F. M. and Ahmed, S. S. (1995) Clin. Biochem. 28:145-154). Other members of the family include pyruvate aminotransferase, branched-chain amino acid aminotransferase, tyrosine aminotransferase, aromatic aminotransferase, alanine:glyoxylate minotransferase (AGT), and kynurenine aminotransferase (Vacca, R. A. et al. (1997) J. Biol. Chem. 272:21932-21937). Kynurenine aminotransferase catalyzes the irreversible transamination of the L-tryptophan metabolite L-kynurenine to form kynurenic acid. The enzyme may also catalyzes the reversible transamination reaction between L-2-aminoadipate and 2-oxoglutarate to produce 2-oxoadipate and L-glutamate. Kynurenic acid is a putative modulator of glutamatergic neurotransmission, thus a deficiency in kynurenine aminotransferase may be associated with pleiotropic effects (Buchli, R. et al. (1995) J. Biol. Chem. 270:29330-29335).

Glycosyl transferases include the mammalian UDP-glucouronosyl transferases, a family of membrane-bound microsomal enzymes catalyzing the transfer of glucouronic acid to lipophilic substrates in reactions that play important roles in detoxification and excretion of drugs, carcinogens, and other foreign substances. Another mammalian glycosyl transferase, mammalian UDP-galactose-ceramide galactosyl transferase, catalyzes the transfer of galactose to ceramide in the synthesis of galactocerebrosides in myelin membranes of the nervous system. Galactosyl transferases are a subset of glycosyl transferases that transfer galactose (Gal) to the terminal N-acetylglucosamine (GlcNAc) oligosaccharide chains that are part of glycoproteins or glycolipids that are free in solution (Kolbinger, F. et al. (1998) J. Biol. Choem. 273:433-440; Amado, M. et al. (1999) Biochini Biophys. Acta 1473:35-53). β1,3-galactosyltransferases form Type I carbohydrate chains with Gal (β1-3) GkcNAc linkages. Known human and mouse β1,3-galactosyltransferases appear to have a short cytosolic domain, a single transmembrane domain, and a catalytic domain with eight conserved regions. (Kolbinger, F. supra and Hennet, T. et al. (1998) J. Biol. Chem. 273:58-65). A variant of a sequence found within mouse UDP-galactose:β-N-acetylglucosamine β1,3-galactosyltransferase-I region 8 is also found in bacterial galactosyltransferases, suggesting that this sequence defines a galactosyltransferase sequence motif (Hennet, T. supra).

Methyl transferases are involved in a variety of pharmacologically important processes. Nicotinamide N-methyl transferase catalyzes the N-methylation of nicotinamides and other pyridines, an important step in the cellular handling of drugs and other foreign compounds. Phenylethanolamine N-methyl transferase catalyzes the conversion of noradrenalin to adrenalin. 6-O-methylguanine-DNA methyl transferase reverses DNA methylation, an important step in carcinogenesis. Uroporphyrin-III C-methyl transferase, which catalyzes the transfer of two methyl groups from S-adenosyl-L-methionine to uroporphyrinogen III, is the first specific enzyme in the biosynthesis of cobalamin, a dietary enzyme whose uptake is deficient in pernicious anemia. Protein-arginine methyl transferases catalyze the posttranslational methylation of arginine residues in proteins, resulting in the mono- and dimethylation of arginine on the guanidino group. Substrates include histones, myelin basic protein, and heterogeneous nuclear ribonucleoproteins involved in mRNA processing, splicing, and transport. Protein-arginine methyl transferase interacts with proteins upregulated by mitogens, with proteins involved in chronic lymphocytic leukemia, and with interferon, suggesting an important role for methylation in cytokine receptor signaling (in, W.-J. et al. (1996) J. Biol. Chem. 271:15034-15044; Abramovich, C. et al. (1997) EMBO J. 16:260-266; and Scott, H. S. et al. (1998) Genomics 48:330-340).

Phospho transferases catalyze the transfer of high-energy phosphate groups and are important in energy-requiring and releasing reactions. The metabolic enzyme creatine kinase catalyzes the reversible phosphate transfer between creatine/creatine phosphate and ATP/ADP. Glycocyamine kinase catalyzes phosphate transfer from ATP to guanidoacetate, and arginine kinase catalyzes phosphate transfer from ATP to arginine. A cysteine-containing active site is conserved in this family (PROSITE:PDOC00103).

Prenyl transferases are heterodimers, consisting of an alpha and a beta subunit, that catalyze the transfer of an isoprenyl group. A particularly important member of this group is the Ras farnesyltransferase (FTase) enzyme, which transfers a farnesyl moiety from cytosolic farnesylpyrophosphate to a cysteine residue at the carboxyl terminus of the Ras oncogene protein. This modification is required to anchor Ras to the cell membrane so that it can perform its role in signal transduction. FTase inhibitors have been shown to be effective in blocking Ras function, and demonstrate antitumor activity in vitro and in vivo (Buolamwini, J. K. (1999) Curr. Opin. Chem. Biol. 3:500-509). FTase shares structural similarity with geranylgeranyl transferase, or Rab GG transferase. This enzyme prenylates Rab proteins, allowing them to perform their roles in regulating vesicle transport (Seabra, M. C. (1996) J. Biol. Chem. 271:14398-14404). The enzyme para-hydroxybenzoate (PHB) polyprenyl diphosphate transferase catalyzes the condensation of PHB and polyprenyl diphosphate in the synthesis of ubiquinone, an essential component of the electron transfer system.

Saccharyl transferases are glycating enzymes involved in a variety of metabolic processes. Oligosacchryl transferase-48, for example, is a receptor for advanced glycation endproducts. Accumulation of these endproducts is observed in vascular complications of diabetes, macrovascular disease, renal insufficiency, and Alzheimer's disease (Thornalley, P. J. (1998) Cell Mol. Biol. (Noisy-Le-Grand) 44:1013-1023).

Coenzyme A (CoA) transferase catalyzes the transfer of CoA between two carboxylic acids. Succinyl CoA:3-oxoacid CoA transferase, for example, transfers CoA from succinyl-CoA to a recipient such as acetoacetate. Acetoacetate is essential to the metabolism of ketone bodies, which accumulate in tissues affected by metabolic disorders such as diabetes (PROSITE: PDOC00980).

NAD:arginine mono-ADP-ribosyltransferases catalyse the transfer of ADP-ribose from NAD to the guanido group of arginine on a target protein. Substrates for these enzymes have been identified in myotubes and activated lymphocytes, and include alpha integrin subunits. These proteins contain characteristic domains involved in NAD binding and ADP-ribose transfer, including a highly acidic region near the carboxy terminus which is required for enzymatic activity (Moss, J. et al (1999) Mol. Cell. Biochem. 193:109-113).

Phosphoribosyltransferases catalyze the synthesis of beta-n-5′-monophosphates from phosphoribosylpyrophosphate and an amine. These enzymes are involved in the biosynthesis of purine and pyrimidine nucleotides, and in the purine and pyrimidine salvage pathways. For example, the enzyme hypoxanthine-guanine phosphoribosyltransferase (HGPRT) is a purine salvage enzyme that catalyzes the conversion of hypoxanthine and guanine to their respective mononucleotides. HGPRT is ubiquitous, is known as a ‘housekeeping’ gene, and is frequently used as an internal control for reverse transcriptase polymerase chain reactions. There is a serine-tyrosine dipeptide that is conserved among all members of the HGPRT family and is essential for the phosphoribosylation of purine bases (Jardim, A. and Ullman, B. (1997) J. Biol. Chem. 272:8967-8973). A partial deficiency of HGPRT can lead to overproduction of uric acid, causing a severe form of gout. An absence of HGPRT causes Lesch-Nyhan syndrome, characterized by hyperuricaemia, mental retardation, choreoathetosis, and compulsive self-mutilation (Sculley, D. G. et al. (1992) Hum. Genet. 90:195-207). Many parasitic organisms are unable to synthesize purines de novo and must rely on the enzymes in salvage pathways for the synthesis of purine nucleotides; thus these enzymes are potential targets for the treatment of parasitic infections (Craig, S. P., and Eakin, A. R. (2000) J. Biol. Chem. 275:20231-20234).

Transglutaminase (Tgases) transferases are Cads dependent enzymes capable of forming isopeptide bonds by catalyzing the transfer of the γ-carboxy group from protein-bound glutamine to the ε-amino group of protein-bound lysine residues or other primary amines. TGases are the enzymes responsible for the cross-linking of cornified envelope (CE), the highly insoluble protein structure on the surface of the corneocytes, into a chemically and mechanically resistant protein polymer. Seven known human Tgases have been identified. Individual transglutaminase gene products are specialized in the cross-linking of specific proteins or tissue structures, such as factor XIIIa which stabilizes the fibrin clot inhemostasis, prostrate transglutaminase which functions in semen coagulation, and tissue transglutaminase which is involved in GTP-binding in receptor signaling. Pour (Tgases 1, 2,3, and X) are expressed in terminally differentiating epithelia such as the epidermis. Tgases are critical for the proper cross-linking of the CE as seen in the pathology of patients suffering from one form of the skin diseases referred to as congenital ichthyosis which has been linked to mutations in the keratinocyte transglutaminase (TGₖ) gene (Nemes, Z. et al., (1999) Proc. Natl. Acad. Sci. U.S.A. 96:8402-8407, Aeschlimann, D. et al., (1998) J. Biol. Chem. 273:3452-3460.)

Lyases

Lyases are a class of enzymes that catalyze the cleavage of C—C, C—O, C—N, C—S, C-(halide), P—O, or other bonds without hydrolysis or oxidation to form two molecules, at least one of which contains a double bond (Stryer, L. (1995) Biochemistry, W.H. Freeman and Co., New York N.Y., p.620). Under the International Classification of Enzymes (Webb, E. C. (1992) Enzyme Nomenclature 1992: Recommendations of the Nomenclature Committee of the International Union of Biochemistry and Molecular Biology on the Nomenclature and Classification of Enzymes, Academic Press, San Diego Calif.), lyases form a distinct class designated by the numeral 4 in the first digit of the enzyme number (i.e., EC 4.x.x x).

Further classification of lyases reflects the type of bond cleaved as well as the nature of the cleaved group. The group of C—C lyases includes carboxyl-lyases (decarboxylases), aldehyde-lyases (aldolases), oxo-acid-lyases, and other lyases. The C—O lyase group includes hydro-lyases, lyases acting on polysaccharides, and other lyases. The C—N lyase group includes ammonia-lyases, amidine-lyases, amine-lyases (deaminases), and other lyases. Lyases are critical components of cellular biochemistry, with roles in metabolic energy production, including fatty acid metabolism and the tricarboxylic acid cycle, as well as other diverse enzymatic processes.

One important family of lyases are the carbonic anhydrases (CA), also called carbonate dehydratases, which catalyze the hydration of carbon dioxide in the reaction H₂O+ [symbol] CO₂. HCO₃⁻+H⁺. CA accelerates this reaction by a factor of over 10⁶ by virtue of a zinc ion located in a deep cleft about 15 Å below the protein's surface and coordinated to the imidazole groups of three His residues. Water bound to the zinc ion is rapidly converted to HCO₃⁻.

Eight enzymatic and evolutionarily related forms of carbonic anhydrase are currently known to exist in humans: three cytosolic isozymes (CAI, CAII, and CAIII), two membrane-bound forms (CAIV and CAVIII), a mitochondrial form (CAV), a secreted salivary form (CAVI) and a yet uncharacterized isozyme (Prosite PDOC00146 Eukaryotic-type carbonic anhydrases signature). Though the isoenzymes CAI, CAII, and bovine CAIII have similar secondary structure and polypeptide-chain fold, CAI has 6 tryptophans, CAII has 7 and CAIII has 8 (Boren, K. et al. (1996) Protein Sci. 5:2479-2484). CAII is the predominant CA isoenzyme in the brain of mammals.

CAs participate in a variety of physiological processes that involve pH regulation, CO₂ and HCO₃⁻; transport, ion transport, and water and electrolyte balance. For example, CAII contributes to H⁺ secretion by gastric parietal cells, by renal tubular cells, and by osteoclasts that secrete H⁺ to acidify the bone-resorbing compartment. In addition, CAII promotes HCO₃⁻ secretion by pancreatic duct cells, cilary body epithelium, choroid plexus, salivary gland acinar cells, and distal colonal epithelium, thus playing a role in the production of pancreatic juice, aqueous humor, cerebrospinal fluid, and saliva, and contributing to electrolyte and water balance. CAII also promotes CO₂ exchange in proximal tubules in the kidney, in erythrocytes, and in lung. CAIV has, roles in several tissues: it facilitates HCO₃⁻ reabsorption in the kidney; promotes CO₂ flux in tissues including brain, skeletal muscle, and heart muscle; and promotes CO₂ exchange from the blood to the alveoli in the lung. CAVI probably plays a role in pH regulation in saliva, along with CAII, and may have a protective effect in the esophagus and stomach. Mitochondrial CAV appears to play important roles in gluconeogenesis and ureagenesis, based on the effects of CA inibbitors on these pathways. (Sly, W. S. and Hu, P. Y. (1995) Ann. Rev. Biochem. 64:375-401.) A number of disease states are marked by variations in CA activity. Mutations in CAII which lead to CAII deficiency are the cause of osteopetrosis with renal tubular acidosis (Online Medelian Inheritance in Man 259730 Osteopetrosis with Renal Tubular Acidosis). The concentration of CAII in the cerebrospinal fluid (CSF) appears to mark disease activity in patients with brain damage. High CA concentrations have been observed in patients with brain infarction. Patients with transient ischemic attack, multiple sclerosis, or epilepsy usually have CAII concentrations in the normal range, but higher CAII levels have been observed in the CSF of those with central nervous system infection, dementia, or trigeminal neuralgia (Parkkila, A. K. et al. (1997) Eur. J. Clin. Invest. 27:392-397). Colonic adenomas and adenocarcinomas have been observed to fail to stain for CA, whereas non-neoplastic controls showed CAI and CAII in the cytoplasm of the columnar cells lining the upper half of colonic crypts. The neoplasms show staining patterns similar to less mature cells lining the base of normal crypts (Gramlich T. L. et al. (1990) Arch. Pathol. Lab. Med. 114:415-419).

Therapeutic interventions in a number of diseases involve altering CA activity. CA inhibitors such as acetazolamide are used in the treatment of glaucoma (Stewart, W. C. (1999) Curr. Opin. Opthamol. 10:99-108), essential tremor and Parkinson's disease (Uitti, R. J. (1998) Geriatrics 53:46-48, 53-57), intermittent ataxia (Singhvi, J. P. et al. (2000) Neurology India 48:78-80), and altitude related illnesses (Klocke, D. L. et al. (1998) Mayo Clin. Proc. 73:988-992).

CA activity can be particularly useful as an indicator of longterm disease condition, since the enzyme reacts relatively slowly to physiological changes. CAI and zinc concentrations have been observed to decrease in hyperthyroid Graves' disease (Yoshida, K. (1996) Tohoku J. Exp. Med. 178:345-356) and glycosylated CAI is observed in diabetes merfitus (Kondo, T. et al. (1987) Clin. Chim. Acta 166:227-236). A positive correlation has been observed between CAI and CAII reactivity and endometriosis (Brinton, D. A. et al. (1996) Ann. Clin. Lab. Sci. 26:409-420; D'Cruz, O. J. et al. (1996) Fertil. Steril. 66:547-556).

Another important member of the lyase family is omithine decarboxylase (ODC), the initial rate-limiting enzyme in polyamine biosynthesis. ODC catalyses the transformation of ornithine into putrescine in the reaction L-ornithine [symbol] putrescine+CO₂. Polyauines, which include putrescine and the subsequent metabolic pathway products spermidine and spermine, are ubiquitous cell components essential for DNA synthesis, cell differentiation, and proliferation. Thus the polyamines play a key role in tumor proliferation (Medina, M. A. et al. (1999) Biochem. Pharmacol. 57:1341-1344).

ODC is a pyridoxal-5′-phosphate (PLP)-dependent enzyme which is active as a homodimer. Conserved residues include those at the PLP binding site and a stretch of glycine residues thought to be part of a substrate binding region (Prosite PDOC00685 Orn/DAP/Arg decarboxylase family 2 signatures). Mammalian ODCs also contain PEST regions, sequence fragments enriched in proline, glutamic acid, serine, and threonine residues that act as signals for intracellular degradation (Medina, sura).

Many chemical carcinogens and tumor promoters increase ODC levels and activity. Several known oncogenes may increase ODC levels by enhancing transcription of the ODC gene, and ODC itself may act as an oncogene when expressed at very high levels. A high level of ODC is found in a number of precancerous conditions, and elevation of ODC levels has been used as part of a screen for tumor-promoing compounds (Pegg, A. E. et al. (1995) J. Cell. Biochem. Suppl. 22:132-138).

Inhibitors of ODC have been used to treat tumors in animal models and human clinical trials, and have been shown to reduce development of tumors of the bladder, brain, esophagus, gastrointestinal tract, lung, oral cavity, mammary gland, stomach, skin and trachea (Pegg, supra; McCann, P. P. and A. E. Pegg (1992) Pharmac. Ther. 54:195-215). ODC also shows promise as a target for chemoprevention (Pegg, supra). ODC inhibitors have also been used to treat infections by African trypanosomes, malaria, and Pneumocystis carinii, and are potentially useful for treatment of autoimmune diseases such as lupus and rheumatoid arthritis (McCann, supra).

Another family of pyridoxal-dependent decarboxylases are the group II decarboxylases. This family includes glutamate decarboxylase (GAD) which catalyzes the decarboxylation of glutamate into the neurotransmitter GABA; histidine decarboxylase (EDC), which catalyzes the decarboxylation of histidine to histamine; aromatic-L-amino-acid decarboxylase (DDC), also known as L-dopa decarboxylase or tryptophan decarboxylase, which catalyzes the decarboxylation of tryptophan to tryptamine and also acts on 5-hydroxy-tryptophan and dihydroxyphenylalanine (L-dopa); and cysteine sulfinic acid decarboxylase (CSD), the rate-limiting enzyme in the synthesis of taurine from cysteine (PROSITE PDOC00329 DDC/GAD/HDC/TyrDC pyridoxal-phosphate attachment site). Taurine is an abundant sulfonic amino acid in brain and is thought to act as an osmoregulator in brain cells (Bitoun, M. and Tappaz, M. (2000) J. Neurochem. 75:919-924).

TNF-Alpha Treatment

Tumor necrosis factor-alpha (TNF-alpha) is a proinflammatory cytokine. It mediates immune regulation and inflammatory responses through various intermediates, including protein kinases, protein phosphatases, reactive oxygen intermediates, phospholipases, proteases, sphingomyelinases and transcription factors. TNF-α-related cytokines generate cellular responses including differentiation, proliferation, cell death, and activation of nuclear factor-κB (NF-κB) (Smith, C. A. et al. (1994) Cell 76:959-962), through its interaction with distinct cell surface receptors (TNRs). NF-κB is a transcription factor that induces genes involved in physiological processes such as response to injury and infection. (For a review of TNF-α in the NF-κB activation pathway see Bowie and O'Neil (2000) Biochem Pharmacol 59:13-23.)

TNF-alpha is upregulated when the endothelium is physically disrupted or functionally perturbed by events such as postischemic reperfusion, acute and chronic inflammation, atherosclerosis, diabetes and chronic arterial hypertension. Inflammatory stimulation sets the stage for later tissue repair. Elevated TNF-alpha initially increases, and then inhibits, the activity of a number of key enzymes including protein-tyrosine kinase (PIKase) and protein-tyrosine phosphatase (Holden, R. J. et al. (1999) Med. Hypotheses 52:319-23).

Development of atherosclerosis involves inflammatory responses induced by circulating lipoprotein. Lipoproteins, such as low-density lipoprotein (LDL), accumulate in the extracellular space of the vascular intima and undergo modifications including oxidation of LDL to Ox-LDL, most avidly in the sub-endothelial space where circulating antioxidant defenses are less effective. Mononuclear phagocytes enter the intima, differentiate into macrophages, and ingest modified lipids including Ox-LDL. During Ox-LDL uptake, macrophages produce cytokines including TNFα, as well as interleukin-1 and growth factors (e.g. M-CSF, VEGF, and PDGF-BB), that elicit further events in atherogenesis such as smooth muscle cell proliferation and production of extracellular matrix by vascular endothelium. These macrophages may also activate genes in endothelium and smooth muscle tissue involved in inflammation and tissue differentiation, including superoxide dismutatse (SOD), IL-8, and ICAM-1.

Non-atherosclerotic vascular endothelium not only mediates vascular dilatation but prevents platelet adhesion and activation, blocks thrombin formation, mitigates fibrin deposition, and attenuates adhesion and transmigration of inflammatory leukocytes. When the endothelium is physically disrupted, or perturbed by events such as postischemic reperfusion, acute and chronic inflammation, atherosclerosis, diabetes and chronic arterial hypertension, it acts in the opposite manner. The perturbed or proinflammatory state is characterised by vaso-constriction, platelet and leukocyte activation and adhesion (involving externalisation, expression and upregulation of, for example, von Willebrand factor, platelet activating factor, P-selectin, ICAM-1, IL-8, MCP-1, and TNF-α), promotion of thrombin formation, coagulation and deposition of fibrin at the vascular wall (expression of tissue factor, PAI-1, and phosphatidyl serine) and, in platelet-leukocyte coaggregates, additional inflammatory interactions via attachment of platelet CD40-ligand to endothelial, monocyte and B-cell CD40. Thrombin formation and inflammatory stimulation set the stage for later tissue repair, but limiting procoagulatory, prothrombotic actions of a dysfunctional vascular endothelium may be the goal of clinical interventions (for review Becker et al. (2000) Z Kardiol 89:160-167).

The discovery of new enzymes, and the polynucleotides encoding them, satisfies a need in the art by providing new compositions which are useful in the diagnosis, prevention, and treatment of immune system disorders, immune deficiencies, developmental disorders, metabolic disorders, smooth muscle disorders, neurological disorders, pulmonary disorders, parasitic infections, and cell proliferative disorders including cancer, and in the assessment of the effects of exogenous compounds on the expression of nucleic acid and amino acid sequences of enzymes.

### SUMMARY OF THE INVENTION

The invention features purified polypeptides, enzymes, referred to collectively as “NZMS” and individually as “NZMS-1,” “NZMS-2,” “NZMS-3,” “NZMS-4,” “NZMS-5,” “NZMS-6,” “NZMS-7,” “NZMS-8,” “NZMS-9,” “NZMS-10,” and “NZMS-11.” In one aspect, the invention provides an isolated polypeptide selected from the group consisting of a) a polypeptide comprising an amino acid sequence selected from the group consisting of SEQ ID NO:1-11, b) a polypeptide comprising a naturally occurring amino acid sequence at least 90% identical to an amino acid sequence selected from the group consisting of SEQ ID NO:1-11, c) a biologically active fragment of a polypeptide having an amino acid sequence selected from the group consisting of SEQ ID NO:1-11, and d) an immunogenic fragment of a polypeptide having an amino acid sequence selected from the group consisting of SEQ ID NO:1-11. In one alternative, the invention provides an isolated polypeptide comprising the amino acid sequence of SEQ ID NO:1-11.

The invention further provides an isolated polynucleotide encoding a polypeptide selected from the group consisting of a) a polypeptide comprising an amino acid sequence selected from the group consisting of SEQ ID NO:1-11, b) a polypeptide comprising a naturally occurring amino acid sequence at least 90% identical to an amino acid sequence selected from the group consisting of SEQ ID NO:1-11, c) a biologically active fragment of a polypeptide having an amino acid sequence selected from the group consisting of SEQ ID NO:1-11, and d) an immunogenic fragment of a polypeptide having an amino acid sequence selected from the group consisting of SEQ ID NO:1-11. In one alternative, the polynucleotide encodes a polypeptide selected from the group consisting of SEQ ID NO:1-11. In another alternative, the polynucleotide is selected from the group consisting of SEQ ID NO:12-22.

Additionally, the invention provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-11, b) a polypeptide comprising a naturally occurring amino acid sequence at least 90% identical to an amino acid sequence selected from the group consisting of SEQ ID NO:1-11, c) a biologically active fragment of a polypeptide having an amino acid sequence selected from the group consisting of SEQ ID NO:1-11, and d) an immunogenic fragment of a polypeptide having an amino acid sequence selected from the group consisting of SEQ ID NO:1-11. In one alternative, the invention provides a cell transformed with the recombinant polynucleotide. In another alternative, the invention provides a transgenic organism comprising the recombinant polynucleotide.

The invention also provides a method for producing a polypeptide selected from the group consisting of a) a polypeptide comprising an amino acid sequence selected from the group consisting of SEQ ID NO:1-11, b) a polypeptide comprising a naturally occurring amino acid sequence at least 90% identical to an amino acid sequence selected from the group consisting of SEQ ID NO:1-11, c) a biologically active fragment of a polypeptide having an amino acid sequence selected from the group consisting of SEQ ID NO:1-11, and d) an immunogenic fragment of a polypeptide having an amino acid sequence selected from the group consisting of SEQ ID NO:1-11. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Additionally, the invention provides an isolated antibody which specifically binds to a polypeptide selected from the group consisting of a) a polypeptide comprising an amino acid sequence selected from the group consisting of SEQ ID NO:1-11, b) a polypeptide comprising a naturally occurring amino acid sequence at least 90% identical to an amino acid sequence selected from the group consisting of SEQ ID NO:1-11, c) a biologically active fragment of a polypeptide having an amino acid sequence selected from the group consisting of SEQ ID NO:1-11, and d) an immunogenic fragment of a polypeptide having an amino acid sequence selected from the group consisting of SEQ ID NO:1-11.

The invention further provides an isolated polynucleotide selected from the group consisting of a) a polynucleotide comprising a polynucleotide sequence selected from the group consisting of SEQ ID NO:12-22, b) a polynucleotide comprising a naturally occurring polynucleotide sequence at least 90% identical to a polynucleotide sequence selected from the group consisting of SEQ ID NO:12-22, c) a polynucleotide complementary to the polynucleotide of a), d) a polynucleotide complementary to the polynucleotide of b), and e) an RNA equivalent of a)-d). In one alternative, the polynucleotide comprises at least 60 contiguous nucleotides.

Additionally, the invention provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO:12-22, b) a polynucleotide comprising a naturally occurring polynucleotide sequence at least 90% identical to a polynucleotide sequence selected from the group consisting of SEQ ID NO:12-22, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex, and optionally, if present, the amount thereof. In one alternative, the probe comprises at least 60 contiguous nucleotides.

The invention further provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO:12-22, b) a polynucleotide comprising a naturally occurring polynucleotide sequence at least 90% identical to a polynucleotide sequence selected from the group consisting of SEQ ID NO:12-22, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof, and, optionally, if present, the amount thereof.

The invention further provides a composition comprising an effective amount of a polypeptide selected from the group consisting of a) a polypeptide comprising an amino acid sequence selected from the group consisting of SEQ ID NO:1-11, b) a polypeptide comprising a naturally occurring amino acid sequence at least 90% identical to an amino acid sequence selected from the group consisting of SEQ ID NO:1-11, c) a biologically active fragment of a polypeptide having an amino acid sequence selected from the group consisting of SEQ ID NO:1-11, and d) an immunogenic fragment of a polypeptide having an amino acid sequence selected from the group consisting of SEQ ID NO:1-11, and a pharmaceutically acceptable excipient In one embodiment, the composition comprises an amino acid sequence selected from the group consisting of SEQ ID NO:1-11. The invention additionally provides a method of treating a disease or condition associated with decreased expression of functional NZMS, comprising administering to a patient in need of such treatment the composition.

The invention also provides a method for screening a compound for effectiveness as an agonist of a polypeptide selected from the group consisting of a) a polypeptide comprising an amino acid sequence selected from the group consisting of SEQ ID NO:1-11, b) a polypeptide comprising a naturally occurring amino acid sequence at least 90% identical to an amino acid sequence selected from the group consisting of SEQ ID NO:1-11, c) a biologically active fragment of a polypeptide having an amino acid sequence selected from the group consisting of SEQ ID NO:1-11, and d) an immunogenic fragment of a polypeptide having an amino acid sequence selected from the group consisting of SEQ ID NO:1-11. The method comprises a) exposing a sample comprising the polypeptide to a compound, and b) detecting agonist activity in the sample. In one alternative, the invention provides a composition comprising an agonist compound identified by the method and a pharmaceutically acceptable excipient. In another alternative, the invention provides a method of treating a disease or condition associated with decreased expression of functional NZMS, comprising administering to a patient in need of such treatment the composition.

Additionally, the invention provides a method for screening a compound for effectiveness as an antagonist of a polypeptide selected from the group consisting of a) a polypeptide comprising an amino acid sequence selected from the group consisting of SEQ ID NO:1-11, b) a polypeptide comprising a naturally occurring amino acid sequence at least 90% identical to an amino acid sequence selected from the group consisting of SEQ ID NO:1-11, c) a biologically active fragment of a polypeptide having an amino acid sequence selected from the group consisting of SEQ ID NO:1-L1, and d) an immunogenic fragment of a polypeptide having an amino acid sequence selected from the group consisting of SEQ ID NO:1-11. The method comprises a) exposing a sample comprising the polypeptide to a compound, and b) detecting antagonist activity in the sample. In one alternative, the invention provides a composition comprising an antagonist compound identified by the method and a pharmaceutically acceptable excipient. In another alternative, the invention provides a method of treating a disease or condition associated with overexpression of functional NZMS, comprising administering to a patient in need of such treatment the composition.

The invention further provides a method of screening for a compound that specifically binds to a polypeptide selected from the group consisting of a) a polypeptide comprising an amino acid sequence selected from the group consisting of SEQ ID NO:1-11, b) a polypeptide comprising a naturally occurring amino acid sequence at least 90% identical to an amino acid sequence selected from the group consisting of SEQ ID NO:1-L1, c) a biologically active fragment of a polypeptide having an amino acid sequence selected from the group consisting of SEQ ID NO:1-11, and d) an immunogenic fragment of a polypeptide having an amino acid sequence selected from the group consisting of SEQ ID NO:1-11. The method comprises a) combining the polypeptide with at least one test compound under suitable conditions, and b) detecting binding of the polypeptide to the test compound, thereby identifying a compound that specifically binds to the polypeptide.

The invention further provides a method of screening for a compound that modulates the activity of a polypeptide selected from the group consisting of a) a polypeptide comprising an amino acid sequence selected from the group consisting of SEQ ID NO:1-11, b) a polypeptide comprising a naturally occurring amino acid sequence at least 90% identical to an amino acid sequence selected from the group consisting of SEQ ID NO:1-11, c) a biologically active fragment of a polypeptide having an amino acid sequence selected from the group consisting of SEQ ID NO:1-11, and d) an immunogenic fragment of a polypeptide having an amino acid sequence selected from the group consisting of SEQ ID NO:1-11. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

The invention further provides a method for screening a compound for effectiveness in altering expression of a target polynucleotide, wherein said target polynucleotide comprises a polynucleotide sequence selected from the group consisting of SEQ ID NO:12-22, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

The invention further provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:12-22, ii) a polynucleotide comprising a naturally occurring polynucleotide sequence at least 90% identical to a polynucleotide sequence selected from the group consisting of SEQ ID NO:12-22, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:12-22, ii) a polynucleotide comprising a naturally occurring polynucleotide sequence at least 90% identical to a polynucleotide sequence selected from the group consisting of SEQ ID NO:12-22, iii) a polynucleotide complementary to the polynucleotide of i), iv) a polynucleotide complementary to the polynucleotide of ii), and v) an RNA equivalent of i)-iv). Alternatively, the target polynucleotide comprises a fragment of a polynucleotide sequence selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for the full length polynucleotide and polypeptide sequences of the present invention.

Table 2 shows the GenBank identification number and annotation of the nearest GenBank homolog for polypeptides of the invention. The probability scores for the matches between each polypeptide and its homolog(s) are also shown.

Table 3 shows structural features of polypeptide sequences of the invention, including predicted motifs and domains, along with the methods, algorithms, and searchable databases used for analysis of the polypeptides.

Table 4 lists the cDNA and/or genomic DNA fragments which were used to assemble polynucleotide sequences of the invention, along with selected fragments of the polynucleotide sequences.

Table 5 shows the representative cDNA library for polynucleotides of the invention.

Table 6 provides an appendix which describes the tissues and vectors used for construction of the cDNA libraries shown in Table 5.

Table 7 shows the tools, programs, and algorithms used to analyze the polynucleotides and polypeptides of the invention, along with applicable descriptions, references, and threshold parameters.

### DESCRIPTION OF THE INVENTION

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“NZMS” refers to the amino acid sequences of substantially purified NZMS obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of NZMS. Agonists may include proteins, nucleic acids, carbohydrates, small molecules, or any other compound or composition which modulates the activity of NZMS either by directly interacting with NZMS or by acting on components of the biological pathway in which NZMS participates.

An “allelic variant” is an alternative form of the gene encoding NZMS. Allelic variants may result from at least one mutation in the nucleic acid sequence and may result in altered mRNAs or in polypeptides whose structure or function may or may not be altered. A gene may have none, one, or many, allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding NZMS include those sequences with deletions, insertions, or substitutions of different nucleotides, resulting in a polypeptide the same as NZMS or a polypeptide with at least one functional characteristic of NZMS. Included within this definition are polymorphisms which may or may not be readily detectable using a particular oligonucleotide probe of the polynucleotide encoding NZMS, and improper or unexpected hybridization to allelic variants, with a locus other than the normal chromosomal locus for the polynucleotide sequence encoding NZMS. The encoded protein may also be “altered,” and may contain deletions, insertions, or substitutions of amino acid residues which produce a silent change and result in a functionally equivalent NZMS. Deliberate amino acid substitutions may be made on the basis of similarity in polarity, charge, solubility, hydrophobicity, hydrophilicity, and/or the amphipathic nature of the residues, as long as the biological or immunological activity of NZMS is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies well known in the art.

The term “antagonist” refers to a molecule which inhibits or attenuates the biological activity of NZMS. Antagonists may include proteins such as antibodies, nucleic acids, carbohydrates, small molecules, or any other compound or composition which modulates the activity of NZMS either by directly interacting with NZMS or by acting on components of the biological pathway in which NZMS participates.

The term “antibody” refers to intact immunoglobulin molecules as well as to fragments thereof, such as Fab, F(ab′)₂, and Fv fragments, which are capable of binding an epitopic determinant. Antibodies that bind NZMS polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH. The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker. (See, e.g., Brody, E. N. and L. Gold (2000) J. Biotechnol. 74:5-13.)

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer”-refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic NZMS, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide sequence” and a “composition comprising a given amino acid sequence” refer broadly to any composition containing the given polynucleotide or amino acid sequence. The composition may comprise a dry formulation or an aqueous solution. Compositions comprising polynucleotide sequences encoding NZMS or fragments of NZMS may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| | |
|---|---|
| Original Residue | Conservative Substitution |

| | |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassorttnent of stable substructures, thus allowing acceleration of the evolution of new protein functions.

A “fragment” is a unique portion of NZMS or the polynucleotide encoding NZMS which is identical in sequence to but shorter in length than the parent sequence. A fragment may comprise up to the entire length of the defined sequence, minus one nucleotide/amino acid residue. For example, a fragment may comprise from 5 to 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:12-22 comprises a region of unique polynucleotide sequence that specifically identifies SEQ ID NO:12-22, for example, as distinct from any other sequence in the genome from which the fragment was obtained. A fragment of SEQ ID NO:12-22 is useful, for example, in hybridization and amplification technologies and in analogous methods that distinguish SEQ ID NO:12-22 from related polynucleotide sequences. The precise length of a fragment of SEQ ID NO:12-22 and the region of SEQ ID NO:12-22 to which the fragment corresponds are routinely determinable by one of ordinary skill in the art based on the intended purpose for the fragment.

A fragment of SEQ ID NO:1-11 is encoded by a fragment of SEQ ID NO:12-22. A fragment of SEQ ID NO:1-11 comprises a region of unique amino acid sequence that specifically identifies SEQ ID NO:1-11. For example, a fragment of SEQ ID NO:1-11 is useful as an immunogenic peptide for the development of antibodies that specifically recognize SEQ ID NO:1-1. The precise length of a fragment of SEQ ID NO:1-11 and the region of SEQ ID NO:1-11 to which the fragment corresponds are routinely determinable by one of ordinary skill in the art based on the intended purpose for the fragment.

A “full length” polynucleotide sequence is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:

Matrix: BLOSUM62

Reward for match: 1

Penalty for mismatch:−2

Open Gap: 5 and Extension Gap: 2 penalties

Gap×drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty-3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:

Matrix: BLOSUM62

Open Gap: 11 and Extension Gap: 1 penalties

Gap×drop-off. 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The This the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of NZMS which is capable of eliciting an immune response when introduced into a living organism, for example, a mammal. The term “immunogenic fragment” also includes any polypeptide or oligopeptide fragment of NZMS which is useful in any of the antibody production methods disclosed herein or known in the art.

The term “microarray” refers to an arrangement of a plurality of polynucleotides, polypeptides, or other chemical compounds on a substrate.

The terms “element” and “array element” refer to a polynucleotide, polypeptide, or other chemical compound having a unique and defined position on a microarray.

The term “modulate” refers to a change in the activity of NZMS. For example, modulation may cause an increase or a decrease in protein activity, binding characteristics, or any other biological, functional, or immunological properties of NZMS.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Teptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an NZMS may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of NZMS.

“Probe” refers to nucleic acid sequences encoding NZMS, their complements, or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989) Molecular Cloning: A Laboratory Manual, 2^{nd} ed., vol. 1-3, Cold Spring Harbor Press, Plainview N.Y.; Ausubel, F. M. et al. (1987) Current Protocols in Molecular Biology, Greene Publ. Assoc. & Wiley-Intersciences, New York N.Y.; Innis, M. et al. (1990) PCR Protocols. A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer 3 primer selection program (available to the public from the Whitehead Institute/MIT Center for Genome Research, Cambridge Mass.) allows the user to input a “mispriming library,” in which sequences to avoid as primer binding sites are user-specified. Primer 3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource: Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemilumninescent, or chromogenic agents; substrates; cofactors; inhibitors; magnetic particles; and other moieties known in the art

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

The term “sample” is used in its broadest sense. A sample suspected of containing NZMS, nucleic acids encoding NZMS, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody win reduce the amount of labeled A that binds to the antibody.

The term “substantially purified” refers to nucleic acid or amino acid sequences that are removed from their natural environment and are isolated or separated, and are at least 60% free, preferably at least 75% free, and most preferably at least 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 07, 1999) set at default parameters. Such a pair of nucleic acids may show, for example, at least 50%, at least 60%, at least 70%, at least 80%, at least 85%, at least 90%, at least 91%, at least 92%, at least 930%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotide sequences that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 07, 1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

### THE INVENTION

The invention is based on the discovery of new human enzymes (NZMS), the polynucleotides encoding NZMS, and the use of these compositions for the diagnosis, treatment, or prevention of immune system disorders, immune deficiencies, developmental disorders, metabolic disorders, smooth muscle disorders, neurological disorders, pulmonary disorders, parasitic infections, and cell proliferative disorders including cancer.

Table 1 summarizes the nomenclature for the full length polynucleotide and polypeptide sequences of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown.

Table 2 shows sequences with homology to the polypeptides of the invention as identified by BLAST analysis against the GenBank protein (genpept) database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog. Column 4 shows the probability scores for the matches between each polypeptide and its homolog(s). Column 5 shows the annotation of the GenBank homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites and potential glycosylation sites, as determined by the MOTIFS program of the GCG sequence analysis software package (Genetics Computer Group, Madison Wis.), as well as amino acid residues comprising signature sequences, domains, and motifs. Column 5 shows analytical methods for protein structure/function analysis and in some cases, searchable databases to which the is analytical methods were applied.

Together, Tables 2 and 3 summarize the properties of polypeptides of the invention, and these properties establish that the claimed polypeptides are enzymes.

For example, SEQ ID NO:3 is 64% identical, from residue P68 to residue S297, to Arabidopsis thaliana para-hydroxy benzoate polyprenyl diphosphate transferase (GenBank ID g12082328) as determined by the Basic Local Alignment Search Tool (BLAST). (See Table 2.) The BLAST probability score is 1.6e-78, which indicates the probability of obtaining the observed polypeptide sequence alignment by chance. SEQ ID NO:3 also contains an UbiA prenyltransferase family domain as determined by searching for statistically significant matches in the hidden Markov model (BMM)-based PFAM database of conserved protein family domains. (See Table 3.)

As another example, SEQ ID NO:4 is 55% identical, from residue Q44 to residue C377, to human beta-1,3-N-acetylglucosaminyltransferase bGnT-3 (GenBank ID g12619296) as determined by the Basic Local Alignment Search Tool (BLAST). (See Table 2.) The BLAST probability score is 7.14e-94, which indicates the probability of obtaining the observed polypeptide sequence alignment by chance. SEQ ID NO:4 also contains a glycosyltransferase domain as determined by searching for statistically significant matches in the hidden Markov model (HMM)-based PFAM database of conserved protein family domains. (See Table 3.)

As another example, SEQ ID NO:5 is 41% identical, from residue P66 to residue V483, to aerobic yeast [Kluyveromyces lactis] D-lactate dehydrogenase (cytochrome) (GenBank ID g⁶02029) as determined by the Basic Local Alignment Search Tool (BLAST). (See Table 2.) The BLAST probability score is 8.9e-87, which indicates the probability of obtaining the observed polypeptide sequence alignment by chance. SEQ ID NO:5 also contains a PAD binding domain as determined by searching for statistically significant matches in the hidden Markov model (HMM)-based PFAM database of conserved protein family domains. (See Table 3.)

As another example, SEQ ID NO:7 is 50% identical, from residue P27 to residue E508, to Oryctolasus cuniculus lactase-phlorizin hydrolase (GenBank ID g415865) as determined by the Basic Local Alignment Search Tool (BLAST). (See Table 2.) The BLAST probability score is 3.1e-131, which indicates the probability of obtaining the observed polypeptide sequence alignment by chance. SEQ ID NO:7 also contains a glycosyl hydrolase family 1 domain as determined by searching for statistically significant matches in the hidden Markov model (HMM)-based PFAM database of conserved protein family domains. (See Table 3.) Data from BLIMPS, PROFILESCAN, MOTIFS, and additional BLAST analyses provide further corroborative evidence that SEQ ID NO:7 is a glycosyl hydrolase.

As another example, SEQ ID NO:8 is 99% identical, from residue M1 to residue G287, to human carbonic anhydrase 14 (GenBank ID g6009640) as determined by the Basic Local Alignment Search Tool (BLAST). (See Table 2.) The BLAST probability score is 4.5e-156, which indicates the probability of obtaining the observed polypeptide sequence alignment by chance. SEQ ID NO:8 also contains a eukaryotic-type carbonic anhydrase domain as determined by searching for statistically significant matches in the hidden Markov model (M)-based PFAM database of conserved protein family domains. (See Table 3.) Data from BLIMPS, PROFILESCAN and additional BLAST analyses provide further corroborative evidence that SEQ ID NO:8 is a carbonic anhydrase.

As another example, SEQ ID NO:9 is 85% identical, from residue M1 to residue L554, to Bos taurus UDP_Gal NAC: polypeptide N-acetylgalactosaminyl transferase (GenBank ID g289412) as determined by the Basic Local Alignment Search Tool (BLAST). (See Table 2.) The BLAST probability score is 4.9e-269, which indicates the probability of obtaining the observed polypeptide sequence alignment by chance. SEQ ID NO:9 also contains a glycosyl transferase domain as determined by searching for statistically significant matches in the hidden Markov model (HMM)-based PFAM database of conserved protein family domains. (See Table 3.) Data from BUMPS and additional BLAST analyses provide further corroborative evidence that SEQ ID NO:9 is a glycosyl transferase.

SEQ ID NO:1-2, SEQ ID NO:6, and SEQ ID NO:10-11 were analyzed and annotated in a similar manner. The algorithms and parameters for the analysis of SEQ ID NO:1-11 are described in Table 7.

As shown in Table 4, the full length polynucleotide sequences of the present invention were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide sequences of the invention, and of fragments of the polynucleotide sequences which are useful, for example, in hybridization or amplification technologies that identify SEQ ID NO:12-22 or that distinguish between SEQ ID NO:12-22 and related polynucleotide sequences.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotide sequences. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}YYYYY_N_{3—}N₄ represents a “stitched” sequence in which XXXXXX is the identification number of the cluster of sequences to which the algorithm was applied, and YYYYY is the number of the prediction generated by the algorithm, and N_{1,2,3} . . . , if present, represent specific exons that may have been manually edited during analysis (See Example V). Alternatively, the polynucleotide fragments in column 2 may refer to assemblages of exons brought together by an “exon-stretching” algorithm For example, a polynucleotide sequence identified as FLXXXXX_gAAAA_gBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).]

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, | Exon prediction from genomic sequences using, for |
| GFG, | example, GENSCAN (Stanford University, CA, USA) |
| ENST | or FGENES (Computer Genomics Group, The Sanger |
| | Centre, Cambridge, UK) |
| GBI | Hand-edited analysis of genomic sequences. |
| FL | Stitched or stretched genomic sequences |
| | (see Example V). |
| INCY | Full length transcript and exon prediction from |
| | mapping of EST sequences to the genome. Genomic |
| | location and EST composition data are combined |
| | to predict the exons and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotide sequences which were assembled using Incyte cDNA sequences. The representative cDNA library is the Incyte cDNA library which is most frequently represented by the Incyte cDNA sequences which were used to assemble and confirm the above polynucleotide sequences. The tissues and vectors which were used to construct the cDNA libraries shown in Table 5 are described in Table 6.

The invention also encompasses NZMS variants. A preferred NZMS variant is one which has at least about 80%, or alternatively at least about 90%, or even at least about 95% amino acid sequence identity to the NZMS amino acid sequence, and which contains at least one functional or structural characteristic of NZMS.

The invention also encompasses polynucleotides which encode NZMS. In a particular embodiment, the invention encompasses a polynucleotide sequence comprising a sequence selected from the group consisting of SEQ ID NO:12-22, which encodes NZMS. The polynucleotide sequences of SEQ ID NO:12-22, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses a variant of a polynucleotide sequence encoding NZMS. In particular, such a variant polynucleotide sequence will have at least about 70%, or alternatively at least about 85%, or even at least about 95% polynucleotide sequence identity to the polynucleotide sequence encoding NZMS. A particular aspect of the invention encompasses a variant of a polynucleotide sequence comprising a sequence selected from the group consisting of SEQ ID NO:12-22 which has at least about 70%, or alternatively at least about 85%, or even at least about 95% polynucleotide sequence identity to a nucleic acid sequence selected from the group consisting of SEQ ID NO:12-22. Any one of the polynucleotide variants described above can encode an amino acid sequence which contains at least one functional or structural characteristic of NZMS.

In addition, or in the alternative, a polynucleotide variant of the invention is a splice variant of a polynucleotide sequence encoding NZMS. A splice variant may have portions which have significant sequence identity to the polynucleotide sequence encoding NZMS, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to the polynucleotide sequence encoding NZMS over its entire length; however, portions of the splice variant will have at least about 70%, or alternatively at least about 85%, or alternatively at least about 95%, or alternatively 100% polynucleotide sequence identity to portions of the polynucleotide sequence encoding NZMS. Any one of the splice variants described above can encode an amino acid sequence which contains at least one functional or structural characteristic of NZMS.

It will be appreciated by those skilled in the art that as a result of the degeneracy of the genetic code, a multitude of polynucleotide sequences encoding NZMS, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring NZMS, and all such variations are to be considered as being specifically disclosed.

Although nucleotide sequences which encode NZMS and its variants are generally capable of hybridizing to the nucleotide sequence of the naturally occurring NZMS under appropriately selected conditions of stringency, it may be advantageous to produce nucleotide sequences encoding NZMS or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding NZMS and its derivatives without altering the encoded amino acid sequences include the production of RNA transcripts having more desirable properties, such as a greater half-life, than transcripts produced from the naturally occurring sequence.

The invention also encompasses production of DNA sequences which encode NZMS and NZMS derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding NZMS or any fragment thereof.

Also encompassed by the invention are polynucleotide sequences that are capable of hybridizing to the claimed polynucleotide sequences, and, in particular, to those shown in SEQ ID NO:12-22 and fragments thereof under various conditions of stringency. (See, e.g., Wabl, G. M. and S. L. Berger (1987) Methods Enzymol. 152:399-407; Kimmel, A. R. (1987) Methods Enzymol. 152:507-511.) Hybridization conditions, including annealing and wash conditions, are described in “Defnitions.”

Methods for DNA sequencing are well known in the art and may be used to practice any of the embodiments of the invention. The methods may employ such enzymes as the Kienow fragment of DNA polymerase I, SEQLTENASE (US Biochemical, Cleveland 011), Taq polymerase (Applied Biosystems), thermostable 17 polymerase (Amersham Pharmacia Biotech, Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 liquid transfer system Hilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Molecular Dynamics, Sunnyvale Calif.), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.) The nucleic acid sequences encoding NZMS may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode NZMS may be cloned in recombinant DNA molecules that direct expression of NZMS, or fragments or functional equivalents thereof, in appropriate host cells. Due to the inherent degeneracy of the genetic code, other DNA sequences which encode substantially the same or a functionally equivalent amino acid sequence may be produced and used to express NZMS.

The nucleotide sequences of the present invention can be engineered using methods generally known in the art in order to alter NZMS-encoding sequences for a variety of purposes including, but not limited to, modification of the cloning, processing, and/or expression of the gene product DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of NZMS, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, sequences encoding NZMS may be synthesized, in whole or in part, using chemical methods well known in the art. (See, e.g., Caruthers, M. R et al. (1980) Nucleic Acids Symp. Ser. 7:215-223; and Horn, T. et al. (1980) Nucleic Acids Symp. Ser. 7:225-232.) Alternatively, NZMS itself or a fragment thereof may be synthesized using chemical methods. For example, peptide synthesis can be performed using various solution-phase or solid-phase techniques. (See, e.g., Creighton, T. (1984) Proteins, Structures and Molecular Properties, WH Freeman, New York N.Y., pp. 55-60; and Roberge, J. Y. et al. (1995) Science 269:202-204.) Automated synthesis may be achieved using the ABI 431A peptide synthesizer (Applied Biosystems). Additionally, the amino acid sequence of NZMS, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and P. Z. Regnier (1990) Methods Enzymol. 182:392-421.) The composition of the synthetic peptides may be confirmed by amino acid analysis or by sequencing. (See, e.g., Creighton, supra, pp. 28-53.)

In order to express a biologically active NZMS, the nucleotide sequences encoding NZMS or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding NZMS. Such elements may vary in their strength and specificity. Specific initiation signals may also be used to achieve more efficient translation of sequences encoding NZMS. Such signals include the ATG initiation codon and adjacent sequences, e.g. the Kozak sequence. In cases where sequences encoding NZMS and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl.

Cell Differ. 20:125-162.) Methods which are well known to those skilled in the art may be used to construct expression vectors containing sequences encoding NZMS and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, P. M. et al. (1995) Current Protocols in Molecular Biology, John Wiley & Sons, New York N.Y., ch. 9, 13, and 16.)

A variety of expression vector/host systems may be utilized to contain and express sequences encoding NZMS. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M. and N. Somia (1997) Nature 389:239-242.) The invention is not limited by the host cell employed.

In bacterial systems, a number of cloning and expression vectors may be selected depending upon the use intended for polynucleotide sequences encoding NZMS. For example, routine cloning, subcloning, and propagation of polynucleotide sequences encoding NZMS can be achieved using a multifunctional E. coli vector such as PBLUESCRIPT (Stratagene, La Jolla Calif.) or PSPORT1 plasmid (Life Technologies). Ligation of sequences encoding NZMS into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of NZMS are needed, e.g. for the production of antibodies, vectors which direct high level expression of NZMS may be used. For example, vectors containing the strong, inducible SP6 or 17 bacteriophage promoter may be used.

Yeast expression systems may be used for production of NZMS. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of NZMS. Transcription of sequences encoding NZMS may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, sequences encoding NZMS may be ligated into an adenovirus transcription/translation complex consisting of the late promoter and tripartite leader sequence. Insertion in a non-essential E1 or E3 region of the viral genome may be used to obtain infective virus which expresses NZMS in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355.)

For long term production of recombinant proteins in mammalian systems, stable expression of NZMS in cell lines is preferred. For example, sequences encoding NZMS can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk and api cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e.g., Hartman, S. C. and R. C. Mulligan (1988) Proc. Natl. Acad. Sci. USA 85:8047-8051.) Visible markers, e.g., anthocyanins, green fluorescent proteins (GFP; Clontech), B glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding NZMS is inserted within a marker gene sequence, transformed cells containing sequences encoding NZMS can be identified by the absence of marker gene function. Alternatively, a marker gene can be placed in tandem with a sequence encoding NZMS under the control of a single promoter. Expression of the marker gene in response to induction or selection usually indicates expression of the tandem gene as well.

In general, host cells that contain the nucleic acid sequence encoding NZMS and that express NZMS may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of NZMS using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on NZMS is preferred, but a competitive binding assay may be employed. These and other assays are well known in the art. (See, e.g., Hampton, R. et al. (1990) Serological Methods, a Laboratory Manual, APS Press, St. Paul M Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding NZMS include oligolabeling, nick translation, end-labeling, or PCR amplification using a labeled nucleotide. Alternatively, the sequences encoding NZMS, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding NZMS may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode NZMS may be designed to contain signal sequences which direct secretion of NZMS through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, NDCK, HBE293, and WI38) are available from the American Type Culture Collection (ATCC, Manassas Va.) and may be chosen to ensure the correct modification and processing of the foreign protein.

In another embodiment of the invention, natural, modified, or recombinant nucleic acid sequences encoding NZMS may be ligated to a heterologous sequence resulting in translation of a fusion protein in any of the aforementioned host systems. For example, a chimeric NZMS protein containing a heterologous moiety that can be recognized by a commercially available antibody may facilitate the screening of peptide libraries for inhibitors of NZMS activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the NZMS encoding sequence and the heterologous protein sequence, so that NZMS may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In a further embodiment of the invention, synthesis of radiolabeled NZMS may be achieved in vitro using the TNT rabbit reticulocyte lysate or wheat germ extract system (Promega). These systems couple transcription and translation of protein-coding sequences operably associated with the 17, T3, or SP6 promoters. Translation takes place in the presence of a radiolabeled amino acid precursor, for example, ³⁵S-methionine.

NZMS of the present invention or fragments thereof may be used to screen for compounds that specifically bind to NZMS. At least one and up to a plurality of test compounds may be screened for specific binding to NZMS. Examples of test compounds include antibodies, oligonucleotides, proteins (e.g., receptors), or small molecules.

In one embodiment, the compound thus identified is closely related to the natural ligand of NZMS, e.g., a ligand or fragment thereof, a natural substrate, a structural or functional mimetic, or a natural binding partner. (See, e.g., Coligan, J. E. et al. (1991) Current Protocols in Immunology 1(2): Chapter 5.) Similarly, the compound can be closely related to the natural receptor to which NZMS binds, or to at least a fragment of the receptor, e.g., the ligand binding site. In either case, the compound can be rationally designed using known techniques. In one embodiment, screening for these compounds involves producing appropriate cells which express NZMS, either as a secreted protein or on the cell membrane. Preferred cells include cells from mammals, yeast, Drosophila, or E. coli. Cells expressing NZMS or cell membrane fractions which contain NZMS are then contacted with a test compound and binding, stimulation, or inhibition of activity of either NZMS or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with NZMS, either in solution or affixed to a solid support, and detecting the binding of NZMS to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

NZMS of the present invention or fragments thereof may be used to screen for compounds that modulate the activity of NZMS. Such compounds may include agonists, antagonists, or partial or inverse agonists. In one embodiment, an assay is performed under conditions permissive for NZMS activity, wherein NZMS is combined with at least one test compound, and the activity of NZMS in the presence of a test compound is compared with the activity of NZMS in the absence of the test compound. A change in the activity of NZMS in the presence of the test compound is indicative of a compound that modulates the activity of NZMS. Alternatively, a test compound is combined with an in vitro or cell-free system comprising NZMS under conditions suitable for NZMS activity, and the assay is performed. In either of these assays, a test compound which modulates the activity of NZMS may do so indirectly and need not come in direct contact with the test compound. At least one and up to a plurality of test compounds may be screened.

In another embodiment, polynucleotides encoding NZMS or their mammalian homologs may be “Knocked out” in an animal model system using homologous recombination in embryonic stem (ES) cells. Such techniques are well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding NZMS may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding NZMS can also be used to create “knockin” humanized animals (pigs) or transgenic animals (mice or rats) to model human disease. With knockin technology, a region of a polynucleotide encoding NZMS is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress NZMS, e.g., by secreting NZMS in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of NZMS and enzymes. In addition, the expression of NZMS is closely associated with brain tissue, kidney tissue, lung tissue, ventricle tissue, esophageal tumor tissue, and prostate tumor tissue. Therefore, NZMS appears to play a role in immune system disorders, immune deficiencies, developmental disorders, metabolic disorders, smooth muscle disorders, neurological disorders, cardiac disorders, pulmonary disorders, parasitic infections, and cell proliferative disorders including cancer. In the treatment of disorders associated with increased NZMS expression or activity, it is desirable to decrease the expression or activity of NZMS. In the treatment of disorders associated with decreased NZMS expression or activity, it is desirable to increase the expression or activity of NZMS.

Therefore, in one embodiment, NZMS or a fragment or derivative thereof may be administered to a subject to treat or prevent a disorder associated with decreased expression or activity of NZMS. Examples of such disorders include, but are not limited to, an immune system disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; an immune deficiency such as acquired immunodeficiency syndrome (AIDS), X-linked agammaglobinemia of Bruton, common variable immunodeficiency (CVI), DiGeorge's syndrome (thymic hypoplasia), thymic dysplasia, isolated IgA deficiency, severe combined immunodeficiency disease (SCBD), immunodeficiency with thrombocytopenia and eczema (Wiskott-Aldrich syndrome), Chediak-Higashi syndrome, chronic granulomatous diseases, hereditary angioneurotic edema, and immunodeficiency associated with Cushing's disease; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; an eye disorder such as ocular hypertension and glaucoma; a metabolic disorder such as Sjoegren-Larsson syndrome (SLS), hyperprolinemia, type II, acute alcohol intoxication, adrenoleukodystrophy, Alport's syndrome, choroideremia, Duchenne and Becker muscular dystrophy, Down's syndrome, cystic fibrosis, chronic granulomatous disease, Gaucher's disease, Huntington's chorea, Marfan's syndrome, muscular dystrophy, myotonic dystrophy, pycnodysostosis, Refsum's syndrome, retinoblastoma, sickle cell anemia, thalassemia, Werner syndrome, von Willebrand's disease, Wilms' tumor, Zellweger syndrome, peroxisomal acyl-CoA oxidase deficiency, peroxisomal thiolase deficiency, peroxisomal bifunctional protein deficiency, mitochondrial carnitine palmitoyl transferase and carnitine deficiency, mitochondrial very-long-chain acyl-CoA dehydrogenase deficiency, mitochondrial medium-chain acyl-CoA dehydrogenase deficiency, mitochondrial short-chain acyl-CoA dehydrogenase deficiency, mitochondrial electron transport flavoprotein and electron transport flavoprotein ubiquinone oxidoreductase deficiency, mitochondrial trifunctional protein deficiency, and mitochondrial short-chain 3-hydroxyacyl-CoA dehydrogenase deficiency; and a smooth muscle disorder such as angina, anaphylactic shock, arrhythmias, asthma, cardiovascular shock, Cushing's syndrome, hypertension, hypoglycemia, myocardial infarction, migraine, and pheochromocytoma, and myopathies including cardiomyopathy, encephalopathy, epilepsy, Kearns-Sayre syndrome, lactic acidosis, myoclonic disorder, and ophthalmoplegia, hyperammonemia, trimethylaminuria (fish-odor syndrome), 3-hydroxydicarboxylic aciduria, dicarboxylic aciduria, xanthinuria, congenital lipoid adrenal hyperplasia (CLAH), albinism type III, hyperinsulinism-hyperammonemia syndrome (HIS), glutaric acidemia type I (GA-I), familial recurrent myoglobinuria, insulin resistance, hereditary thymine-uraciluria (familial pyrimidinemia), idiopathic sidereoblastic anemia (AISA), neonatal adrenoleukodystrophy, hypoxia, increased damage to tissues caused by trauma, radiation and ultraviolet exposure, liver dysfunction, marked obesity, methemoglobinemia (HM1, HM2, and HM3), hypertrophic hirsutism with amenorrhea, and Hermansky-Pudlack syndrome, Reye's syndrome, hypoketotic hypoglycemia, isovaleric acidemia, and chronic hemolytic anemia; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including karu, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, familial frontotemporal dementia, and Lesch-Nyan syndrome; a cardiovascular disorder such as Raynaud's disease, aneurysms, arterial dissections, varicose veins, thrombophlebitis and phlebothrombosis, vascular, tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation; a pulmonary disorder such as congenital lung anomalies, atelectasis, pulmonary congestion and edema; pulmonary embolism, pulmonary hemorrhage, pulmonary infarction, pulmonary hypertension, vascular sclerosis, obstructive pulmonary disease, restrictive pulmonary disease, chronic obstructive pulmonary disease, emphysema, chronic bronchitis, bronchial asthma, bronchiectasis, bacterial pneumonia, viral and mycoplasmal pneumonia, lung abscess, pulmonary tuberculosis, diffuse interstitial diseases, pneumoconioses, sarcoidosis, idiopathic pulmonary fibrosis, desquamative interstitial pneumonitis, hypersensitivity pneumonitis, pulmonary eosinophilia bronchiolitis obliterans-organizing pneumonia, diffuse pulmonary hemorrhage syndromes, Goodpastare's syndromes, idiopathic pulmonary hemosiderosis, pulmonary involvement in collagen-vascular disorders, pulmonary alveolar proteinosis, lung tumors, inflammatory and noninflammatory pleural effusions, pneumothorax, pleural tumors, drug-induced lung disease, radiation-induced lung disease, and complications of lung transplantation; an infection by parasites classified as plasmodium or malaria-causing, parasitic entamoeba, leishmania, trypanosoma, toxoplasma, pneumocystis carinii, intestinal protozoa such as giardia, trichomonas, tissue nematodes such as trichinella, intestinal nematodes such as ascaris, lymphatic filarial nematodes, trematodes such as schistosoma, and cestodes (tapeworm); and a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycytiemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus.

In another embodiment, a vector capable of expressing NZMS or a fragment or derivative thereof may be administered to a subject to treat or prevent a disorder associated with decreased expression or activity of NZMS including, but not limited to, those described above.

In a further embodiment, a composition comprising a substantially purified NZMS in conjunction with a suitable pharmaceutical carrier may be administered to a subject to treat or prevent a disorder associated with decreased expression or activity of NZMS including, but not limited to, those provided above.

In still another embodiment, an agonist which modulates the activity of NZMS may be administered to a subject to treat or prevent a disorder associated with decreased expression or activity of NZMS including, but not limited to, those listed above.

In a further embodiment, an antagonist of NZMS may be administered to a subject to treat or prevent a disorder associated with increased expression or activity of NZMS. Examples of such disorders include, but are not limited to, those immune system disorders, immune deficiencies, developmental disorders, metabolic disorders, neurological disorders, pulmonary disorders, parasitic infections, and cell proliferative disorders including cancer described above. In one aspect, an antibody which specifically binds NZMS may be used directly as an antagonist or indirectly as a targeting or delivery mechanism for bringing a pharmaceutical agent to cells or tissues which express NZMS.

In an additional embodiment, a vector expressing the complement of the polynucleotide encoding NZMS may be administered to a subject to treat or prevent a disorder associated with increased expression or activity of NZMS including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of NZMS may be produced using methods which are generally known in the art. In particular, purified NZMS may be used to produce antibodies or to screen libraries of pharmaceutical agents to identify those which specifically bind NZMS. Antibodies to NZMS may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with NZMS or with any fragment or oligopeptide thereof which has immunogenic properties. Depending on the host species, various adjuvants may be used to increase immunological response. Such adjuvants include, but are not limited to, Preund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to NZMS have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of NZMS amino acids may be fused with those of another protein, such as KLH, and antibodies to the chimeric molecule may be produced.

Monoclonal antibodies to NZMS may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce NZMS-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for NZMS may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between NZMS and its specific antibody. A two-site, monoclonal-based immunoassay utilizing monoclonal antibodies reactive to two non-interfering NZMS epitopes is generally used, but a competitive binding assay may also be employed (Pound, supra).

Various methods such as Scatchard analysis in conjunction with radioimmunoassay techniques may be used to assess the affinity of antibodies for NZMS. Affinity is expressed as an association constant, Kₐ which is defined as the molar concentration of NZMS-antibody complex divided by the molar concentrations of free antigen and free antibody under equilibrium conditions. The Kₐ determined for a preparation of polyclonal antibodies, which are heterogeneous in their affinities for multiple NZMS epitopes, represents the average affinity, or avidity, of the antibodies for NZMS. The Kₐ determined for a preparation of monoclonal antibodies, which are monospecific for a particular NZMS epitope, represents a true measure of affinity. High-affinity antibody preparations with Kₐ ranging from about 10⁹ to 10¹² L/mole are preferred for use in immunoassays in which the NZMS-antibody complex must withstand rigorous manipulations. Low-affinity antibody preparations with Kₐ ranging from about 10⁶ to 10⁷ L/mole are preferred for use in immunopurification and similar procedures which ultimately require dissociation of NZMS, preferably in active form, from the antibody (Catty, D. (1988) Antibodies. Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of NZMS-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, the polynucleotides encoding NZMS, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding NZMS. Such technology is well known in the art, and antisense oligonucleotides or larger fragments can be designed from various locations along the coding or control regions of sequences encoding NZMS. (See, e.g., Agrawal, S., ed. (1996) Antisense Therapeutics, Humana Press Inc., Totawa N.J..)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E. et al. (1998) J. Allergy Clin. Immunol 102(3):469-475; and Scanlon, K. J. et al. (1995) 9(13):1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Pharmacol. Ther. 63(3):323-347.) Other gene delivery mechanisms include liposome-derived systems, artificial viral envelopes, and other systems known in the art. (See, e.g., Rossi, J. J. (1995) Br. Med. Bull. 51(1):217-225; Boado, R. J. et al. (1998) J. Pharm. Sci. 87(11):1308-1315; and Morris, M. C. et al. (1997) Nucleic Acids Res. 25(14):2730-2736.)

In another embodiment of the invention, polynucleotides encoding NZMS may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, famrial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (BBV, HCV); fungal parasites, such as Candida albicans and Paracoccidioides brasiliensis; and protozoan parasites such as Plasmodium falciparum and Trypanosoma cruzi). In the case where a genetic deficiency in NZMS expression or regulation causes disease, the expression of NZMS from an appropriate population of transduced cells may alleviate the clinical manifestations caused by the genetic deficiency.

In a further embodiment of the invention, diseases or disorders caused by deficiencies in NZMS are treated by constructing mammalian expression vectors encoding NZMS and introducing these vectors by mechanical means into NZMS-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J-L. and H. Récipon (1998) Curr. Opin. Biotechnol. 9:445-450).

Expression vectors that may be effective for the expression of NZMS include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo. Alto Calif.). NZMS may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and R Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid Invitrogen)); the ecdysone-inducible promoter (available in the plasmids PVGRXR and PMND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding NZMS from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, B. et al. (1982) EMBO J. 1:841-845). The introduction of DNA to primary cells requires modification of these standardized mammalian transfection protocols.

In another embodiment of the invention, diseases or disorders caused by genetic defects with respect to NZMS expression are treated by constructing a retrovirus vector consisting of (i) the polynucleotide encoding NZMS under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In the alternative, an adenovirus-based gene therapy delivery system is used to deliver polynucleotides encoding NZMS to cells which have one or more genetic abnormalities with respect to the expression of NZMS. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and N. Somia (1997) Nature 18:389:239-242, both incorporated by reference herein.

In another alternative, a herpes-based, gene therapy delivery system is used to deliver polynucleotides encoding NZMS to target cells which have one or more genetic abnormalities with respect to the expression of NZMS. The use of herpes simplex virus (HSV)-based vectors may be especially valuable for introducing NZMS to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another alternative, an alphavirus (positive, single-stranded RNA virus) vector is used to deliver polynucleotides encoding NZMS to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for NZMS into the alphavirus genome in place of the capsid-coding region results in the production of a large number of NZMS-coding RNAs and the synthesis of high levels of NZMS in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of NZMS into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing, Mt. Kisco N.Y., pp.163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding NZMS.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding NZMS. Such DNA sequences may be incorporated into a wide variety of vectors with suitable RNA polymerase promoters such as 17 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding NZMS. Compounds which may be effective in altering expression of a specific polynucleotide may include, but are not limited to, oligonucleotides, antisense oligonucleotides, triple helic-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased NZMS expression or activity, a compound which specifically inhibits expression of the polynucleotide encoding NZMS may be therapeutically useful, and in the treatment of disorders associated with decreased NZMS expression or activity, a compound which specifically promotes expression of the polynucleotide encoding NZMS may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding NZMS is exposed to at least one test compound thus obtained. The sample may comprise, for example, an intact or permeabilized cell, or an in vitro cell-free or reconstituted biochemical system. Alterations in the expression of a polynucleotide encoding NZMS are assayed by any method commonly known in the art. Typically, the expression of a specific nucleotide is detected by hybridization with a probe having a nucleotide sequence complementary to the sequence of the polynucleotide encoding NZMS. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of NZMS, antibodies to NZMS, and mimetics, agonists, antagonists, or inhibitors of NZMS.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising NZMS or fragments thereof. For example, liposome preparations containing a cell-impermeable macromolecule may promote cell fusion and intracellular delivery of the macromolecule. Alternatively, NZMS or a fragment thereof may be joined to a short cationic N-terminal portion from the MV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example NZMS or fragments thereof, antibodies of NZMS, and agonists, antagonists or inhibitors of NZMS, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the ID₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000/g, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind NZMS may be used for the diagnosis of disorders characterized by expression of NZMS, or in assays to monitor patients being treated with NZMS or agonists, antagonists, or inhibitors of NZMS. Antibodies useful for diagnostic purposes may be prepared in the same manner as described above for therapeutics. Diagnostic assays for NZMS include methods which utilize the antibody and a label to detect NZMS in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring NZMS, including ELISAs, RIAs, and FACS, are known in the art and provide a basis for diagnosing altered or abnormal levels of NZMS expression. Normal or standard values for NZMS expression are established by combining body fluids or cell extracts taken from normal mammalian subjects, for example, human subjects, with antibodies to NZMS under conditions suitable for complex formation. The amount of standard complex formation may be quantitated by various methods, such as photometric means. Quantities of NZMS expressed in subject, control, and disease samples from biopsied tissues are compared with the standard values. Deviation between standard and subject values establishes the parameters for diagnosing disease.

In another embodiment of the invention, the polynucleotides encoding NZMS may be used for diagnostic purposes. The polynucleotides which may be used include oligonucleotide sequences, complementary RNA and DNA molecules, and PNAs. The polynucleotides may be used to detect and quantify gene expression in biopsied tissues in which expression of NZMS may be correlated with disease. The diagnostic assay may be used to determine absence, presence, and excess expression of NZMS, and to monitor regulation of NZMS levels during therapeutic intervention.

In one aspect, hybridization with PCR probes which are capable of detecting polynucleotide sequences, including genomic sequences, encoding NZMS or closely related molecules may be used to identify nucleic acid sequences which encode NZMS. The specificity of the probe, whether it is made from a highly specific region, e.g., the 5′regulatory region, or from a less specific region, e.g., a conserved motif, and the stringency of the hybridization or amplification will determine whether the probe identifies only naturally occurring sequences encoding NZMS, allelic variants, or related sequences.

Probes may also be used for the detection of related sequences, and may have at least 50% sequence identity to any of the NZMS encoding sequences. The hybridization probes of the subject invention may be DNA or RNA and may be derived from the sequence of SEQ ID NO:12-22 or from genomic sequences including promoters, enhancers, and introns of the NZMS gene.

Means for producing specific hybridization probes for DNAs encoding NZMS include the cloning of polynucleotide sequences encoding NZMS or NZMS derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding NZMS may be used for the diagnosis of disorders associated with expression of NZMS. Examples of such disorders include, but are not limited to, an immune system disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophliha, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; an immune deficiency such as acquired immunodeficiency syndrome (AIDS), X-linked agammaglobinemia of Bruton, common variable immunodeficiency (CVI), DiGeorge's syndrome (thymic hypoplasia), thymic dysplasia, isolated IgA deficiency, severe combined immunodeficiency disease (SCID), immunodeficiency with thrombocytopenia and eczema (Wiskott-Aldrich syndrome), Chediak-Higashi syndrome, chronic granulomatous diseases, hereditary angioneurotic edema, and immunodeficiency associated with Cushing's disease; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; an eye disorder such as ocular hypertension and glaucoma; a metabolic disorder such as Sjoegren-Larsson syndrome (SLS), hyperprolinemia, type II, acute alcohol intoxication, adrenoleukodystrophy, Alport's syndrome, choroideremia, Duchenne and Becker muscular dystrophy, Down's syndrome, cystic fibrosis, chronic granulomatous disease, Gaucher's disease, Huntington's chorea, Marfan's syndrome, muscular dystrophy, myotonic dystrophy, pycnodysostosis, Refsum's syndrome, retinoblastoma, sickle cell anemia, thalassemia, Werner syndrome, von Willebrand's disease, Wilms' tumor, Zellweger syndrome, peroxisomal acyl-CoA oxidase deficiency, peroxisomal thiolase deficiency, peroxisomal bifunctional protein deficiency, mitochondrial carnitine palmitoyl transferase and carnitine deficiency, mitochondrial very-long-chain acyl-CoA dehydrogenase deficiency, mitochondrial medium-chain acyl-CoA dehydrogenase deficiency, mitochondrial short-chain acyl-CoA dehydrogenase deficiency, mitochondrial electron transport flavoprotein and electron transport flavoprotein-ubiquinone oxidoreductase deficiency, mitochondrial trifunctional protein deficiency, and mitochondrial short-chain 3-hydroxyacyl-CoA dehydrogenase deficiency; and a smooth muscle disorder such as angina, anaphylactic shock, arrhythmias, asthma, cardiovascular shock, Cushing's syndrome, hypertension, hypoglycemia, myocardial infarction, migraine, and pheochromocytoma, and myopathies including cardiomyopathy, encephalopathy, epilepsy, Kearns-Sayre syndrome, lactic acidosis, myoclonic disorder, and ophthalmoplegia, hyperammonemia, trimethylaminuria (fish-odor syndrome), 3-hydroxydicarboxylic aciduria, dicarboxylic aciduria, xanthinuria, congenital lipoid adrenal hyperplasia (CLAH, albinism type m, hyperinsulinism-hyperammonemia syndrome (BHS), glutaric acidemia type I (GA-I), familial recurrent myoglobinuria, insulin resistance, hereditary thymine-uraciluria (familial pyrimidinemia), idiopathic sidereoblastic anemia (AISA), neonatal adrenoleukodystrophy, hypoxia, increased damage to tissues caused by trauma, radiation and ultraviolet exposure, liver dysfunction, marked obesity, methemoglobinemia (HM1, HM2, and HM3), hypertrophic hirsutism with amenorrhea, and Hermansky-Pudlack syndrome, Reye's syndrome, hypoketotic hypoglycemia, isovaleric acidemia, and chronic hemolytic anemia; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, familial frontotemporal dementia, and Lesch-Nyan syndrome; a cardiovascular disorder such as Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation; a pulmonary disorder such as congenital lung anomalies, atelectasis, pulmonary congestion and edema, pulmonary embolism, pulmonary hemorrhage, pulmonary infarction, pulmonary hypertension, vascular sclerosis, obstructive pulmonary disease, restrictive pulmonary disease, chronic obstructive pulmonary disease, emphysema, chronic bronchitis, bronchial asthma, bronchiectasis, bacterial pneumonia, viral and mycoplasmal pneumonia, lung abscess, pulmonary tuberculosis, diffuse interstitial diseases, pneumoconioses, sarcoidosis, idiopathic pulmonary fibrosis, desquamative interstitial pneumonitis, hypersensitivity pneumonitis, pulmonary eosinophilia bronchiolitis obliterans-organizing pneumonia, diffuse pulmonary hemorrhage syndromes, Goodpasture's syndromes, idiopathic pulmonary hemosiderosis, pulmonary involvement in collagen-vascular disorders, pulmonary alveolar proteinosis, lung tumors, inflammatory and noninflammatory pleural effusions, pneumothorax, pleural tumors, drug-induced lung disease, radiation-induced lung disease, and complications of lung transplantation; an infection by parasites classified as plasmodium or malaria-causing, parasitic entamoeba, leishmania, trypanosoma, toxoplasma, pneumocystis carinii, intestinal protozoa such as giardia, trichomonas, tissue nematodes such as trichinella, intestinal nematodes such as ascaris, lymphatic filarial nematodes, trematodes such as schistosoma, and cestodes (tapeworm); and a cell proliferative disorder such as actinic keratosis, arteriosclerosis, atherosclerosis, bursitis, cirrhosis, hepatitis, mixed connective tissue disease (MCID), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. The polynucleotide sequences encoding NZMS may be used in Southern or northern analysis, dot blot, or other membrane-based technologies; in PCR technologies; in dipstick, pin, and multiformat ELISA-like assays; and in microarrays utilizing fluids or tissues from patients to detect altered NZMS expression. Such qualitative or quantitative methods are well known in the art.

In a particular aspect, the nucleotide sequences encoding NZMS may be useful in assays that detect the presence of associated disorders, particularly those mentioned above. The nucleotide sequences encoding NZMS may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding NZMS in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of NZMS, a normal or standard profile for expression is established. This may be accomplished by combining body fluids or cell extracts taken from normal subjects, either animal or human, with a sequence, or a fragment thereof, encoding NZMS, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding NZMS may involve the use of PCR. These oligomers may be chemically synthesized, generated enzymatically, or produced in vitro. Oligomers will preferably contain a fragment of a polynucleotide encoding NZMS, or a fragment of a polynucleotide complementary to the polynucleotide encoding NZMS, and will be employed under optimized conditions for identification of a specific gene or condition. Oligomers may also be employed under less stringent conditions for detection or quantification of closely related DNA or RNA sequences.

In a particular aspect, oligonucleotide primers derived from the polynucleotide sequences encoding NZMS may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from the polynucleotide sequences encoding NZMS are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (is SNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectromety using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations. (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641.) Methods which may also be used to quantify the expression of NZMS include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotide sequences described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, NZMS, fragments of NZMS, or antibodies specific for NZMS may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471, expressly incorporated by reference herein). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http://www.niehs.nih.gov/oc/news/toxchip.htm.) Therefore, it is important and desirable in toxicological screening using toxicant signatures to include all expressed gene sequences.

In one embodiment, the toxicity of a test compound is assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another particular embodiment relates to the use of the polypeptide sequences of the present invention to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of the present invention. In some cases, further sequence data may be obtained for definitive protein identification.

A proteomic profile may also be generated using antibodies specific for NZMS to quantify the levels of NZMS expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Micro arrays: A Practical Approach, M. Schena, ed. (1999) Oxford University Press, London, hereby expressly incorporated by reference.

In another embodiment of the invention, nucleic acid sequences encoding NZMS may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (YACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.) Once mapped, the nucleic acid sequences of the invention may be used to develop genetic linkage maps, for example, which correlate the inheritance of a disease state with the inheritance of a particular chromosome region or restriction fragment length polymorphism (RFLP). (See, for example, Lander, E. S. and D. Botstein (1986) Proc. Natl. Acad. Sci. USA 83:7353-7357.) Fluorescent in situ hybridization (FISH) may be correlated with other physical and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding NZMS on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, NZMS, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between NZMS and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with NZMS, or fragments thereof, and washed. Bound NZMS is then detected by methods well known in the art. Purified NZMS can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding NZMS specifically compete with a test compound for binding NZMS. In this manner, antibodies can be used to detect the presence of any peptide which shares one or more antigenic determinants with NZMS.

In additional embodiments, the nucleotide sequences which encode NZMS may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The disclosures of all patents, applications, and publications mentioned above and below, including U.S. Ser. No. 60/268,113, U.S. Ser. No. 60/269,215, U.S. Ser. No. 60/272,271, U.S. Ser. No. 60/274,091, U.S. Ser. No. 60/274,423, U.S. Ser. No. 60/278,480, and U.S. Ser. No. 60/278,479, are hereby expressly incorporated by reference.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Life Technologies), PCDNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Life Technologies.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWEIL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Pharmacia Biotech or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM; and HMM-based protein domain databases such as SMART (Schultz et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families. See, for example, Eddy, S. R. (1996) Curr. Opin. Struct. Biol. 6:361-365.) The queries were performed using programs based on BLAST, FASTA, BLMPS, and HMMER. The Incyte cDNA sequences were assembled to produce full length polynucleotide sequences. Alternatively, Genank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding fall length polypeptide sequences. Alternatively, a polypeptide of the invention may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM; and HMM-based protein domain databases such as SMART. Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and fall length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:12-22. Fragments from about 20 to about 4000 nucleotides which are useful in hybridization and amplification technologies are described in Table 4, column 2.

IV. Identification and Editing of Coding Sequences from Genomic DNA

Putative enzymes were initially identified by running the Genscan gene identification program against public genomic sequence databases (e.g., gbpri and gbhtg). Genscan is a general-purpose gene identification program which analyzes genomic DNA sequences from a variety of organisms (See Burge, C. and S. Karlin (1997) J. Mol. Biol 268:78-94, and Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode enzymes, the encoded polypeptides were analyzed by querying against PFAM models for enzymes. Potential enzymes were also identified by homology to Incyte cDNA sequences that had been annotated as enzymes. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Pull length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomic Sequence Data with cDNA Sequence Data “Stitched” Sequences

Partial cDNA sequences were extended with exons predicted by the Genscan gene identification program described in Example IV. Partial cDNAs assembled as described in Example m were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a fall length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of NZMS Encoding Polynucleotides

The sequences which were used to assemble SEQ ID NO:12-22 were compared with sequences from the Incyte LIFESEQ database and public domain databases using BLAST and other implementations of the Smith-Waterman algorithm. Sequences from these databases that matched SEQ ID NO:12-22 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel (1995) supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotide sequences encoding NZMS are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding NZMS. cDNA sequences and cDNA library/tissue information are found in the LIFESEQ GOLD database (Incyte Genomics, Palo Alto Calif.).

VIII. Extension of NZMS Encoding Polynucleotides

Full length polynucleotide sequences were also produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄₁ and 2-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Pharmacia Biotech), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2×carb liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Applied Biosystems).

In like manner, full length polynucleotide sequences are verified using the above procedure or are used to obtain 5′ regulatory sequences using the above procedure along with oligonucleotides designed for such extension, and an appropriate genomic library.

IX. Identification of Single Nucleotide Polymorphisms in NZMS Encoding Polynucleotides

Common DNA sequence variants known as single nucleotide polymorphisms (SNPs) were identified in SEQ ID NO:12-22 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:12-22 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²p] adenosine triphosphate (Amersham Pharmacia Biotech), and T4 polynucleotide kinase (DuPont NEN, Boston Mass.). The labeled oligonucleotides are substantially purified using a SEPHADEX G-25 superfine size exclusion dextran bead column (Amersham Pharmacia Biotech). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu I (DuPont NEN).

The DNA from each digest is fractionated on a 0.7% agarose gel and transferred to nylon membranes (Nytran Plus, Schleicher & Schuell, Durham N.Mex. Hybridization is carried out for 16 hours at 40° C. To remove nonspecific signals, blots are sequentially washed at room temperature under conditions of up to, for example, 0.1×saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing, See, e.g., Baldeschweiler, supra.), mechanical microspotting technologies, and derivatives thereof. The substrate in each of the aforementioned technologies should be uniform and solid with a non-porous surface (Schena (1999), supra). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements. (See, e.g., Schena, M. et al. (1995) Science 270:467-470; Shalon, D. et al. (1996) Genome Res. 6:639-645; Marshall, A. and J. Hodgson (1998) Nat. Biotechnol. 16:27-31.)

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Bach poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Pharmacia Biotech). The reverse transcription reaction is performed in a 25 ml volume containing 200 ng poly(A)⁺ RNA with GEMBRIGHT kits (Incyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Pharmacia Biotech).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Micro arrays are UV-crosslinked using a STRATALINKER UV-crosslinker (Stratagene). Microarrays are washed at room temperature once in 0.2% SDS and three times in distilled water. Non-specific binding sitesare blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

For SEQ ID NO:21, for example, CASMCs were maintained in SmGM-2 medium containing 5% fetal bovine serum (FBS), recombinant hEGF (0.5 ng.ml⁻¹), insulin (5 ng.ml⁻¹), hFGF-B (4 ng.ml⁻¹), Gentamicin (50 μg.ml⁻¹), and Amphotericin-B (50 ng.ml⁻¹) (as supplied by Clonetics, San Diego Calif.), at 37° C. in a 5% CO₂ atmosphere. The cells were grown to 85% confluency and then treated with TNF-α (10 ng.ml⁻¹) for 1, 2, 4, 6, 8, 10, 24, and 48 hours. These TNF-α treated cells were compared to untreated CASMCs collected at 85% confluency (t=0 hour). In this manner, it was demonstrated that the presence of TNF alpha alters expression in vascular tissue of component 2191340 of SEQ ID NO:21 by a factor of at least 2.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte).

XIII. Complementary Polynucleotides

Sequences complementary to the NZMS-encoding sequences, or any parts thereof, are used to detect, decrease, or inhibit expression of naturally occurring NZMS. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of NZMS. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the NZMS-encoding transcript.

XIII. Expression of NZMS

Expression and purification of NZMS is achieved using bacterial or virus-based expression systems. For expression of NZMS in bacteria, cDNA is subcloned into an appropriate vector containing an antibiotic resistance gene and an inducible promoter that directs high levels of cDNA transcription. Examples of such promoters include, but are not limited to, the tp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express NZMS upon induction with isopropyl beta-D-thiogalactopyranoside (IPTG). Expression of NZMS in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding NZMS by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, NZMS is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Pharmacia Biotech). Following purification, the GST moiety can be proteolytically cleaved from NZMS at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified NZMS obtained by these methods can be used directly in the assays shown in Examples XVII, XVIII and XIX, where applicable.

XIV. Functional Assays

NZMS function is assessed by expressing the sequences encoding NZMS at physiologically elevated levels in mammalian cell culture systems. cDNA is subcloned into a mammalian expression vector containing a strong promoter that drives high levels of cDNA expression. Vectors of choice include PCMV SPORT (Life Technologies) and PCR3.1 (Invitrogen, Carlsbad Calif.), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of NZMS on gene expression can be assessed using highly purified populations of cells transfected with sequences encoding NZMS and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding NZMS and other genes of interest can be analyzed by northern analysis or microarray techniques.

XV. Production of NZMS Specific Antibodies

NZMS substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the NZMS amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, ch. 11.) Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide and anti-NZMS activity by, for example, binding the peptide or NZMS to a substrate, blocking with 1% BSA, reacting with rabbit antisera, washing, and reacting with radio-iodinated goat anti-rabbit IgG.

XVI. Purification of Naturally Occurring NZMS Using Specific Antibodies

Naturally occurring or recombinant NZMS is substantially purified by immunoaffinity chromatography using antibodies specific for NZMS. An immunoaffinity column is constructed by covalently coupling anti-NZMS antibody to an activated chromatographic resin, such as CNBr-activated SEPHAROSE (Amersham Pharmacia Biotech). After the coupling, the resin is blocked and washed according to the manufacturer's instructions.

Media containing NZMS are passed over the immunoaffinity column, and the column is washed under conditions that allow the preferential absorbance of NZMS (e.g., high ionic strength buffers in the presence of detergent). The column is eluted under conditions that disrupt antibody/NZMS binding (e.g., a buffer of pH 2 to pH 3, or a high concentration of a chaotrope, such as urea or thiocyanate ion), and NZMS is collected.

XVII. Identification of Molecules Which Interact with NZMS

NZMS, or biologically active fragments thereof, are labeled with ¹²⁵I Bolton-Hunter reagent (See, e.g., Bolton, A. E. and W. M. Hunter (1973) Biochem. J. 133:529-539.) Candidate molecules previously arrayed in the wells of a multi-well plate are incubated with the labeled NZMS, washed, and any wells with labeled NZMS complex are assayed. Data obtained using different concentrations of NZMS are used to calculate values for the number, affinity, and association of NZMS with the candidate molecules.

Alternatively, molecules interacting with NZMS are analyzed using the yeast two-hybrid system as described in Fields, S. and O. Song (1989) Nature 340:245-246, or using commercially available kits based on the two-hybrid system, such as the MATCHMAKER system (Clontech).

NZMS may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of NZMS Activity

NZMS activity is demonstrated through a variety of specific enzyme assays, some of which are outlined below.

NZMS activity can be measured spectrophotometrically by determining the amount of solubilized RNA that is produced as a result of incubation of RNA substrate with NZMS. 5 μl (20 μg) of a 4 mg/ml solution of yeast tRNA (Sigma) is added to 0.8 ml of 40 mM sodium phosphate, pH 7.5, containing NZMS. The reaction is incubated at 25° C. for 15 minutes. The reaction is stopped by addition of 0.5 ml of an ice-cold fresh solution of 20 mM lanthanum nitrate plus 3% perchloric acid. The stopped reaction is incubated on ice for at least 15 min, and the insoluble tRNA is removed by centrifugation for 5 min at 10,000 g. Solubilized tRNA is determined as UV absorbance (260 nm) of the remaining supernatant, with A₂₆₀ of 1.0 corresponding to 40 μg of solubilized RNA (Rosenberg, H. F. et al. (1996) Nucleic Acids Research 24:3507-3513).

NZMS activity in the hydrolytic direction is performed spectroscopically by measuring the rate of the product (homocysteine) formed by reaction with 5,5′-Dithiobis(2-nitrobenzoic acid) (DTNB). To 800 It of an enzyme solution containing 4.7 μg of NZMS-1 and 4 units of adenosine deaminase in 50 mM potassium phosphate buffer, pH 7.2, containing 1 mM EDTA (buffer A), is added 200 μl of S-Adenosyl-L-homocysteine (500 μM) containing 250 μM DTNB in buffer A. The reaction mixture is incubated at 37° C. for 2 minutes. Hydrolytic activity is monitored at 412 nm continuously using a diode array UV spectrophotometer. Enzyme activity is defined as the amount of enzyme that can hydrolyze 1 μmol of S-Adenosyl-L-homocysteine/minute (Yuan, C—S et al. (1996) J. Biol. Chem. 271:28009-28015).

Alternatively, NZMS activity can be measured in the synthetic direction as the production of S-adenosyl homocysteine using 3-deazaadenosine as a substrate (Sganga, M. W. et al. supra). Briefly, NZMS-1 is incubated in a 100 μl volume containing 0.1 mM 3-deazaadenosine, 5 nM homocysteine, 20 mM Hepes (pH 7.2). The assay mixture is incubated at 37° C. for 15 minutes. The reaction is terminated by the addition of 10 μl of 3 M perchloric acid. After incubation on ice for 15 minutes, the mixture is centrifuged for 5 minutes at 18,000×g in a microcentrifuge at 4° C. The supernatant is removed, neutralized by the addition of 1 M potassium carbonate, and centrifuged again. A 50 μl aliquot of supernatant is then chromatographed on an Altex Ultrasphere ODS column (5 μm particles, 4.6×250 mm) by isocratic elution with 0.2 M ammonium dihydrogen phosphate (Aldrich) at a flow rate of 1 ml/min. Protein is determined by the bicinchoninic acid assay (Pierce).

Alternatively, NZMS activity can be measured in the synthetic direction by a TLC method (Hershfield, M. S. et al. (1979) J. Biol. Chem. 254:22-25). In a preincubation step, 50 μM [8-¹⁴C]adenosine is incubated with 5 molar equivalents of NAD⁺ for 15 minutes at 22° C. Assay samples containing NZMS in a 50 μl final volume of 50 mM potassium phosphate buffer, pH 7.4, 1 mM DTT, and 5 mM homocysteine, are mixed with the preincubated [8-¹⁴C]adenosine/NAD⁺ to initiate the reaction. The reaction is incubated at 37° C., and 4 μl samples are spotted on TLC plates at 5 minute intervals for 30 minutes. The chromatograms are developed in butanol-1/glacial acetic acid/water (12:3:5, v/v) and dried. Standards are used to identify substrate and products under ultraviolet light. The complete spots containing [¹⁴C]adenosine and [¹⁴C]SAH are then detected by exposing x-ray film to the TLC plate. The radiolabeled substrate and product are then cut from the chromatograms and counted by liquid scintillation spectrometry. Specific activity of the enzyme is determined from the linear least squares slopes of the product vs time plots and the milligrams of protein in the sample (Bethin, K. E. et al. (1995) J. Biol. Chem 270:20698-20702).

NZMS transferase activity is measured through assays such as a methyl transferase assay in which the transfer of radiolabeled methyl groups between a donor substrate and an acceptor substrate is measured (Bokar, J. A. et al. (1994) J. Biol. Chem. 269:17697-17704). Reaction mixtures (50 μl final volume) contain 15 mM HEPES, pH 7.9, 1.5 mM MgCl₂, 10 mM dithiothreitol, 3% polyvinylalcohol, 1.5 μCi [methyl-³H]AdoMet (0.375 μM AdoMet) (DuPont-NEN), 0.6 μg HEM, and acceptor substrate (0.4 μg [³⁵S]RNA or 6-mercaptopurine (6-MP) to 1 nM final concentration). Reaction mixtures are incubated at 30° C. for 30 minutes, then 65° C. for 5 minutes. The products are separated by chromatography or electrophoresis and the level of methyl transferase activity is determined by quantification of methyl-3H recovery.

Lysophosphatidic acid acyltransferase activity of NZMS is measured by incubating samples containing NZMS with 1 mM of e thiol reagent 5,5′-dithiobis(2-nitrobenzoic acid) (DTNB), 50 μm LPA, and 50 μm acyl-CoA in 100 mM Tris-HCl, pH 7.4. The reaction is initiated by addition of acyl-CoA, and allowed to reach equilibrium. Transfer of the acyl group from acyl-CoA to LPA releases free CoA, which reacts with DTNB. The product of the reaction between DTNB and free CoA absorbs at 413 nm. The change in absorbance at 413 nm is measured using a spectrophotometer, and is proportional to the lysophosphatidic acid acyltransferase activity of NZMS in the sample.

N-acyltransferase activity of NZMS is measured using radiolabeled amino acid substrates and measuring radiolabel incorporation into conjugated products. NZMS is incubated in a reaction buffer containing an unlabeled acyl-CoA compound and radiolabeled amino acid, and the radiolabeled acyl-conjugates are separated from the unreacted amino acid by extraction into n-butanol or other appropriate organic solvent. For example, Johnson, M. R. et al. (1990; J. Biol. Chem. 266:10227-10233) measured bile acid-CoA:amino acid N-acyltransferase activity by incubating the enzyme with cholyl-CoA and ³H-glycine or ³H-taurine, separating the tritiated cholate conjugate by extraction into n-butanol, and measuring the radioactivity in the extracted product by scintillation. Alternatively, N-acyltransferase activity is measured using the spectrophotometric determination of reduced CoA (CoASH) described below.

N-acetyltransferase activity of NZMS is measured using the transfer of radiolabel from [¹⁴C]acetyl-CoA to a substrate molecule (for example, see Deguchi, T. (1975) J. Neurochem. 24:1083-5). Alternatively, a newer spectrophotometric assay based on DTNB reaction with CoASH may be used. Free thiol-containing CoASH is formed during N-acetyltransferase catalyzed transfer of an acetyl group to a substrate. CoASH is detected using the absorbance of DTNB conjugate at 412 nm (De Angelis, J. et at. (1997) J. Biol. Chem. 273:3045-3050). NZMS activity is proportional to the rate of radioactivity incorporation into substrate, or the rate of absorbance increase in the spectrophotometric assay.

Aminotransferase activity of NZMS is measured by determining the activity of purified NZMS or crude samples containing NZMS toward various amino and oxo acid substrates under single turnover conditions by monitoring the changes in the UV/VIS absorption spectrum of the enzyme-bound cofactor, PLP. The reactions are performed at 25° C. in 50 mM 4-methybmorpholine (pH 7.5) containing 9 μM purified NZMS or NZMS containing samples and substrate to be tested (amino and oxo acid substrates). The half-reaction from amino acid to oxo acid is followed by measuring the decrease in absorbance at 360 nm and the increase in absorbance at 330 nm due to the conversion of enzyme-bound PLP to PMP. The specificity and relative activity of NZMS is determined by the activity of the enzyme preparation against specific substrates (Vacca, R. A. et al. (1997) J. Biol. Chem. 272:21932-21937).

Galactosyltransferase activity of NZMS is determined by measuring the transfer of galactose from UDP-galactose to a GlcNAc-terminated oligosaccharide chain in a radioactive assay. (Kolbinger, F. et al (1998) J. Biol. Chem. 273:58-65.) The NZMS sample is incubated with 14 μof assay stock solution (180 mM sodium cacodylate, pH 6.5, 1 mg/ml bovine serum albumin, 0.26 mM UDP-galactose, 2 μl of UDP-[³H]galactose), 1 μl of MnCl₂ (500 mM), and 2.5 μl of GlcNAcβO—(CH₂)_{B}—CO₂Me (37 mg/ml in dimethyl sulfoxide), for 60 minutes at 37° C. The reaction is quenched by the addition of 1 ml of water and loaded on a C18 Sep-Pak cartridge (Waters), and the column is washed twice with 5 ml of water to remove unreacted UDP-[³H]galactose. The [³H]galactosylated GkcNAcβO—(CH₂)₈—CO₂Me remains bound to the column during the water washes and is eluted with 5 ml of methanol. Radioactivity in the eluted material is measured by liquid scintillation counting and is proportional to galactosyltransferase activity of NZMS in the starting sample.

Phosphoribosyltransferase activity of NZMS is measured as the transfer of a phosphoribosyl group from phosphoribosylpyrophosphate (PRPP) to a purine or pyrimidine base. Assay mixture (20 μl) containing 50 mM Tris acetate, pH 9.0, 20 mM 2-mercaptoethanol, 12.5 mM MgCl₂, and 0.1 mM labeled substrate, for example, [¹⁴C]uracil, is mixed with 20 μl of NZMS diluted in 0.1 M Tris acetate, pH 9.7, and 1 mg/ml bovine serum albumin. Reactions are preheated for 1 min at 37° C., initiated with 10 μl of 6 mM PRPP, and incubated for 5 min at 37° C. The reaction is stopped by heating at 100° C. for 1 min. The product [¹⁴C]UMP is separated from [¹⁴C]uracil on DEAE-cellulose paper (Turner, R. J. et al. (1998) J. Biol. Chem. 273:5932-5938). The amount of [¹⁴C]UMP produced is proportional to the phosphoribosyltransferase activity of NZMS.

ADP-ribosyltransferase activity of NZMS is measured as the transfer of radiolabel from adenine-NAD to agmatine (Weng, B. et al. (1999) J. Biol. Chem. 274:31797-31803). Purified NZMS is incubated at 30° C. for 1 hr in a total volume of 300 μl containing 50 mM potassium phosphate (pH. 7.5), 20 mM agmatine, and 0.1 mM [adenine-U-¹⁴C]NAD (0.05 mCi). Samples (100 μl) are applied to Dowex columns and [¹⁴C]ADP-ribosylagmatine eluted with 5 ml of water for liquid scintillation counting. The amount of radioactivity recovered is proportional to ADP-ribosyltransferase activity of NZMS.

Aldo/keto reductase activity of NZMS is proportional to the decrease in absorbance at 340 nm as NADPH is consumed (or increased absorbance if NADPH is produced, i.e., if the reverse reaction is monitored). A standard reaction mixture is 135 mM sodium phosphate buffer (pH 6.2-7.2 depending on enzyme), 0.2 mM NADPH, 0.3 M lithium sulfate, 0.5-2.5 mg NZMS and an appropriate level of substrate. The reaction is incubated at 30° C. and the reaction is monitored continuously with a spectrophotometer. NZMS activity is calculated as mol NADPH consumed/mg of NZMS.

Acyl-CoA dehydrogenase activity of NZMS is measured using an anaerobic electron transferring flavoprotein (ETF) assay. The reaction mixture comprises 50 mM Tris-HCl (pH 8.0), 0.5% glucose, and 50 μM acyl-CoA substrate (i.e., isovaleryl-CoA) that is pre-warmed to 32° C. The mixture is depleted of oxygen by repeated exposure to vacuum followed by layering with argon. Trace amounts of oxygen are removed by the addition of glucose oxidase and catalase followed by the addition of ETF to a final concentration of 1 μM. The reaction is initiated by addition of purified NZMS or a sample containing NZMS and exciting the reaction at 342 nm. Quenching of fluorescence caused by the transfer of electron from the substrate to ETF is monitored at 496 nm. 1 unit of acyl-CoA dehydrogenase activity is defined as the amount of NZMS required to reduce 1 μmol of ETF per minute (Reinard, T. et al. (2000) J. Biol. Chem. 275:33738-33743).

Alcohol dehydrogenase activity of NZMS is measured by following the conversion of NAD⁺ to NADH at 340 nm (ε₃₄₀=6.22 mM⁻¹ cm⁻¹) at 25° C. in 0.1 M potassium phosphate (pH 7.5), 0.1 M glycine (pH 10.0), and 2.4 mM NAD⁺. Substrate (e.g., ethanol) and NZMS are then added to the reaction. The production of NADH results in an increase in absorbance at 340 nm and correlates with the oxidation of the alcohol substrate and the amount of alcohol dehydrogenase activity in the NZMS sample (Svensson, S. (1999) J. Biol. Chem. 274:29712-29719).

Aldehyde dehydrogenase activity of NZMS is measured by determining the total hydrolase+dehydrogenase activity of NZMS and subtracting the hydrolase activity. Hydrolase activity is first determined in a reaction mixture containing 0.05 M Tris-HCl (pH 7.8), 100 mM 2-mercaptoethanol, and 0.5-18 μM substrate, e.g., 10-HCO—HPteGlu (10-formyltetrahydrofolate; HPteGlu, tetrahydrofolate) or 10-FDDF (10-formyl-5,8-dideazafolate). Approximately 1 μg of NZMS is added in a final volume of 1.0 mL The reaction is monitored and read against a blank cuvette, containing all components except enzyme. The appearance of product is measured at either 295 nm for 5,8-dideazafolate or 300 nm for HPteGlu using molar extinction coefficients of 1.89×10⁴ and 2.17×10⁴ for 5,8-dideazafolate and HPteGlu, respectively. The addition of NADP⁺ to the reaction mixture allows the measurement of both dehydrogenase and hydrolase activity (assays are performed as before). Based on the production of product in the presence of NADP⁺ and the production of product in the absence of the cofactor, aldehyde dehydrogenase activity is calculated for NZMS. In the alternative, aldehyde dehydrogenase activity is assayed using propanal as substrate. The reaction mixture contains 60 mM sodium pyrophosphate buffer (pH 8.5), 5 mM propanal, 1 mM NADP⁺, and NZMS in a total volume of 1 ml. Activity is determined by the increase in absorbance at 340 nm, resulting from the generation of NADPH, and is proportional to the aldehyde dehydrogenase activity in the sample (Krupenko, S. A. et al. (1995) J. Biol. Chem. 270:519-522).

6-phosphogluconate dehydrogenase activity of NZMS is measured by incubating purified NZMS, or a composition comprising NZMS, in 120 mM triethanolamine (pH 7.5), 0.1 mM EDTA, 0.5 mM NADP⁺, and 10-150 μM 6-phosphogluconate as substrate at 20-25° C. The production of NADPH is measured fluorimetrically (340 nm excitation, 450 nm emission) and is indicative of 6-phosphogluconate dehydrogenase activity. Alternatively, the production of NADPH is measured photometrically, based on absorbance at 340 nm. The molar amount of NADPH produced in the is reaction is proportional to the 6-phosphogluconate dehydrogenase activity in the sample (Tetaud, E. et al. (1999) Biochem. J. 338:55-60).

Ribonucleotide diphosphate reductase activity of NZMS is determined by incubating purified NZMS, or a composition comprising NZMS, along with dithiothreitol, Mg⁺⁺, and ADP, GDP, CDP, or UDP substrate. The product of the reaction, the corresponding deoxyribonucleotide, is separated from the substrate by thin-layer chromatography. The reaction products can be distinguished from the reactants based on rates of migration. The use of radiolabeled substrates is an alternative for increasing the sensitivity of the assay. The amount of deoxyribonucleotides produced in the reaction is proportional to the amount of ribonucleotide diphosphate reductase activity in the sample (note this is true only for pre-steady state kinetic analysis of ribonucleotide diphosphate reductase activity, as the enzyme is subject to negative feedback inhibition by products) (Nutter, L. M. and Y.-C. Cheng (1984) Pharmac. Ther. 26:191-207).

Dihydrodiol dehydrogenase activity of NZMS is measured by incubating purified NZMS, or a composition comprising NZMS, in a reaction mixture comprising 50 mM glycine (pH 9.0), 2.3 mM NADP⁺, 8% DMSO, and a trans-dihydrodiol substrate, selected from the group including but not limited to, (±)-trans-naphthalene-1,2-dihydrodiol, (±)-trans-phenanthrene-1,2-dihydrodiol, and (±)-trans-chrysene-1,2-dihydrodiol. The oxidation reaction is monitored at 340 nm to detect the formation of NADPH, which is indicative of the oxidation of the substrate. The reaction mixture can also be analyzed before and after the addition of NZMS by circular dichroism to determine the stereochemistry of the reaction components and determine which enantiomers of a racemic substrate composition are oxidized by the NZMS (Penning, T. M. (1993) Chemico-Biological Interactions 89:1-34).

Glutathione S-transferase (GST) activity of NZMS is determined by measuring the NZMS catalyzed conjugation of GSH with 1-chloro-2,4-dinitrobenzene (CDNB), a common substrate for most GSTs. NZMS is incubated with 1 mM CDNB and 2.5 mM GSH together in 0.1M potassium phosphate buffer, pH 6.5, at 25° C. The conjugation reaction is measured by the change in absorbance at 340 nm using an ultraviolet spectrophometer. NZMS activity is proportional to the change in absorbance at 340 nm.

15-oxoprostaglandin 13-reductase (PGR) activity of NZMS is measured following the separation of contaminating 15-hydroxyprostaglandin dehydrogenase (15-PGDH) activity by DEAE chromatography. Following isolation of PGR containing fractions (or using the purified NZMS), activity is assayed in a reaction comprising 0.1 M sodium phosphate (pH 7.4), 1 mM 2-mercaptoethanol, 20 μg substrate (e.g., 15-oxo derivatives of prostaglandins PGE₁, PGE₂, and PGE_{2α}), and 1 mM NADH (or a higher concentration of NADPH). NZMS is added to the reaction which is then incubated for 10 min at 37° C. before termination by the addition of 0.25 ml 2 N NaOH. The amount of 15-oxo compound remaining in the sample is determined by measuring the maximum absorption at 500 nm of the terminated reaction and comparing this value to that of a terminated control reaction that received no NZMS. 1 unit of enzyme is defined as the amount required to catalyze the oxidation of 1 μmol substrate per minute and is proportional to the amount of PGR activity in the sample.

Choline dehydrogenase activity of NZMS is identified by the ability of E. coli, transformed with an NZMS expression vector, to grow on media containing choline as the sole carbon and nitrogen source. The ability of the transformed bacteria to thrive is indicative of choline dehydrogenase activity (Magne Øster\dot{a}fs, M. (1998) Proc. Natl. Acad. Sci. USA 95:11394-11399).

An assay for carbonic anhydrase activity of NZMS uses the fluorescent pH indicator 8-hydroxypyrene-1,3,6-trisulfonate (pyranine) in combination with stopped-flow fluorometry to measure carbonic anhydrase activity (Shingles, et al. 1997, Anal. Biochem. 252: 190-197). A pH 6.0 solution is mixed with a pH 8.0 solution and the initial rate of bicarbonate dehydration is measured. Addition of carbonic anhydrase to the pH 6.0 solution enables the measurement of the initial rate of activity at physiological temperatures with resolution times of 2 ms. Shingles et al. used this assay to resolve differences in activity and sensitivity to sulfonamides by comparing mammalian carbonic anhydrase isoforms. The fluorescent technique's sensitivity allows the determination of initial rates with a protein concentration as little as 65 ng/ml.

Protein phosphatase (PP) activity can be measured by the hydrolysis of P-nitrophenyl phosphate (PNPP). NZMS is incubated together with PNPP in HEPES buffer pH 7.5, in the presence of 0.1% β-mercaptoethanol at 37° C. for 60 min. The reaction is stopped by the addition of 6 ml of 10 N NaOH (Diamond, R. H. et al. (1994) Mol. Cell. Biol. 14:3752-62). Alternatively, acid phosphatase activity of NZMS is demonstrated by incubating NZMS containing extract with 100 μl of 10 mM PNPP in 0.1 M sodium citrate, pH 4.5, and 50 μl of 40 mM NaCl at 37° C. for 20 min. The reaction is stopped by the addition of 0.5 ml of 0.4 M glycine/NaOH, pH 10.4 (Saftig, P. et al. (1997) J. Biol. Chem. 272:18628-18635). The increase in light absorbance at 410 nm resulting from the hydrolysis of PNPP is measured using a spectrophotometer. The increase in light absorbance is proportional to the activity of NZMS in the assay.

In the alternative, NZMS activity is determined by measuring the amount of phosphate removed from a phosphorylated protein substrate. Reactions are performed with 2 or 4 nM NZMS in a final volume of 30 μl containing 60 mM Tris, pH 7.6, 1 mM EDTA, 1 mM EDTA, 0.1% 2-mercaptoethanol and 10 μM substrate, ³²P-labeled on serine/threonine or tyrosine, as appropriate. Reactions are initiated with substrate and incubated at 30° C. for 10-15 min. Reactions are quenched with 450 μl of 4% (w/v) activated charcoal in 0.6 M HCl, 90 mM Na₄P₂O₇, and 2 mM NaH₂PO₄, then centrifuged at 12,000×g for 5 min. Acid-soluble ³²Pi is quantified by liquid scintillation counting (Sinclair, C. et al (1999) J. Biol. Chem. 274:23666-23672).

NZMS activity can be determined as the ability of NZMS to cleave ³²P internally labeled T. thermophila pre-tRNA^{Gln}. NZMS and substrate are added to reaction vessels and reactions are carried out in MBB buffer (50 mM Tris-HCl (pH 7.5), 10 mM MgCl₂) for 1 hour at 37° C. Reactions are terminated with the addition of an equal volume of sample loading buffer (SIB: 40 mM EDTA, 8 M urea, 0.2% xylene cyanol, and 0.2% bromophenol blue). The reaction products are separated by electrophoresis on 8 M urea, 6% polyacrylaride gels and analyzed using detection instruments and software capable of quantification of the products. One unit of NZMS activity is defined as the amount of enzyme required to cleave 10% of 28 fmol of T. thermophila pre-tRNA^{Gln} to mature products in 1 hour at 37° C. (True, H. L. et al. (1996) J. Biol. Chem. 271:16559-16566).

Alternatively, cleavage of ³²P internally labeled substrate tRNA by NZMS can be determined in a 20 μl reaction mixture containing 30 mM HEPES-KOH (pH 7.6), 6 mM MgCl₂, 30 mM Kcl, 2 mM DTT, 25 μg/ml bovine serum albumin, 1 unit/μl rRNasin, and 5,000-50,000 cpm of gel-purified substrate RNA. 3.0 μl of NZMS is added to the reaction mixture, which is then incubated at 37° C. for 30 minutes. The reaction is stopped by guanidinium/phenol extraction, precipitated with ethanol in the presence of glycogen, and subjected to denaturing polyacrylamide gel electrophoresis (6 or 8% polyacrylamide, 7 M urea) and autoradiography (Rossmanith, W. et al. (1995) J. Biol. Chem. 270:12885-12891). The NZMS activity is proportional to the amount of cleavage products detected.

XIX. Identification of NZMS Inhibitors

Compounds to be tested are arrayed in the wells of a multi-well plate in varying concentrations along with an appropriate buffer and substrate, as described in the assays in Example XVII. NZMS activity is measured for each well and the ability of each compound to inhibit NZMS activity can be determined, as well as the dose-response profiles. This assay could also be used to identify molecules which enhance NZMS activity.

Various modifications and variations of the described methods and systems of the invention will be apparent to those skilled in the art without departing from the scope and spirit of the invention. Although the invention has been described in connection with certain embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1

| | | | Poly- | Incyte |
|---|---|---|---|---|
| Incyte | Polypeptide | Incyte | nucleotide | Polynucleotide |
| Project ID | SEQ ID NO: | Polypeptide ID | SEQ ID NO: | ID |
| 7484737 | 1 | 7484737CD1 | 12 | 7484737CB1 |
| 7485242 | 2 | 7485242CD1 | 13 | 7485242CB1 |
| 2900469 | 3 | 2900469CD1 | 14 | 2900469CB1 |
| 6928818 | 4 | 6928818CD1 | 15 | 6928818CB1 |
| 1801591 | 5 | 1801591CD1 | 16 | 1801591CB1 |
| 2257558 | 6 | 2257558CD1 | 17 | 2257558CB1 |
| 5701733 | 7 | 5701733CD1 | 18 | 5701733CB1 |
| 2706884 | 8 | 2706884CD1 | 19 | 2706884CB1 |
| 4974616 | 9 | 4974616CD1 | 20 | 4974616CB1 |
| 70861047 | 10 | 70861047CD1 | 21 | 70861047CB1 |
| 7472794 | 11 | 7472794CD1 | 22 | 7472794CB1 |

TABLE 2

| | | GenBank ID NO: | | |
|---|---|---|---|---|
| Polypeptide SEQ | Incyte | or PROTEOME | Probability | |
| ID NO: | Polypeptide ID | ID NO: | Score | Annotation |
| 1 | 7484737CD1 | g2852125 | 7.00E−252 | [Homo sapiens] S-adenosyl homocysteine hydrolase homolog |
| 2 | 7485242CD1 | g4688966 | 2.00E−07 | [Niviventer cremoriventer] pancreatic ribonuclease |
| | | | | Dubois, J. Y. et al. (1999) Mol. Phylogenet. Evol. 13: 181-192 |
| 3 | 2900469CD1 | g12082328 | 1.60E−78 | (AB052553) para-hydroxy bezoate polyprenyl diphosphate transferase |
| | | | | [Arabidopsis thaliana] |
| | | | | Okada, K., et al. (1996) Polyprenyl diphosphate synthase essentially defines the |
| | | | | length of the side chain of ubiquinone. Biochim Biophys Acta. 1302: 217-23 |
| 4 | 6928818CD1 | g12619296 | 7.10E−94 | [Homo sapiens] (AB049585) beta-1,3-N-acetylglucosaminyltransferase bGnT-3 |
| | | | | (Shiraishi, N. et al. (2001) Identification and Characterization of Three Novel |
| | | | | beta1,3-N-Acetylglucosaminyltransferases Structurally Related to the beta1,3- |
| | | | | Galactosyltransferase Family. |
| | | | | J. Biol. Chem. 276, 3498-3507.) |
| 5 | 1801591CD1 | g17740510 | 1.00E−128 | FAD dependent oxidoreductase [Agrobacterium tumefaciens str. C58 (Dupont)] |
| | | | | Lodi, T., et al. (1994) Carbon catabolite repression in Kluyveromyces lactis: |
| | | | | isolation and characterization of the KIDLD gene encoding the mitochondrial |
| | | | | enzyme D-lactate ferricytochrome c oxidoreductase. |
| | | | | Mol. Gen. Genet. 244 (6), 622-629 |
| | | g602029 | 8.90E−87 | [Kluyveromyces lactis] D-lactate dehydrogenase (cytochrome) |
| | | | | Lodi, T. et al. (1994) Mol. Gen. Genet. 244: 622-629 |
| | | | | Carbon catabolite repression in Kluyveromyces lactis: isolation and |
| | | | | characterization of the KIDLD gene encoding the mitochondrial enzyme D-lactate |
| | | | | Ferricytochrome c oxidoreductase |
| 6 | 2257558CD1 | g3647337 | 1.10E−13 | [Schizosaccharomyces pombe] putative trna-splicing endonuclease subunit |
| 7 | 5701733CD1 | g415865 | 3.10E−131 | [Oryctolagus cuniculus] lactase-phlorizin hydrolase |
| | | | | Villa, M. et al. (1993) FEBS Lett. 336: 70-74 |
| 8 | 2706884CD1 | g6009640 | 4.50E−156 | [Homo sapiens] carbonic anhydrase 14 |
| | | | | Fujikawa-Adachi, K. et al. (1999) Genomics 61: 74-81 |
| 9 | 4974616CD1 | g289412 | 4.90E−269 | [Bos taurus] UDP-GalNAc: polypeptide, N-acetylgalactosaminyltransferase |
| | | | | Homa, F. L., et al. (1993) J. Biol. Chem. 268: 12609-12616 |
| 10 | 70861047CD1 | g10336504 | 1.70E−209 | [Homo sapiens] UDP-GalNAc: polypeptide N-acetylgalactosaminyltransferase |
| | | | | Toba, S., et al. (2000) Biochim. Biophys. Acta 1493: 264-268 |
| 11 | 7472794CD1 | g2865607 | 5.90E−37 | [Bos taurus] aralkyl acyl-CoA: amino acid N-acyltransferase |
| | | | | Vessey, D. A. and Lau, E. (1996) J. Biochem. Toxicol. 11: 211-215 |
| | | | | TITLE Determination of the sequence of the aralkyl acyl-CoA: amino acid N- |
| | | | | acyltransferase from bovine liver mitochondria. |
| | | g3004445 | 9.40E−21 | [Bos taurus] arylacetyl acyl-CoA N-acyltransferase |
| | | | | Vessey, D. A. and Lau, E. (1998) |
| | | | | J. Biochem. Mol. Toxicol. 12: 275-279 |

TABLE 3

| | | Amino | | |
|---|---|---|---|---|
| SEQ | Incyte | Acid | | Analytical Methods |
| ID NO: | Polypeptide ID | Residues | Signature Sequences, Domains and Motifs | and Databases |
| 1 | 7484737CD1 | 564 | S-adenosyl-L-homocysteine hydrolase: C140-A506 | HMMER_PFAM |
| | | | S-adenosyl-L-homocysteine hydrolase proteins | BLIMPS_BLOCKS |
| | | | BL00738: 1468-N505, V515-Y564, F139-A178, G179-G203, A204-D241, N258- | |
| | | | Y272, G284-C306, M310-M341, S369-R390, L391-L443 | |
| | | | HYDROLASE ADENOSYL HOMOCYSTEINASE ADOHCYASE NAD ONE- | BLAST_PRODOM |
| | | | CARBON METABOLISM S-ADENOSYL-L-HOMOCYSTEINE PUTATIVE | |
| | | | S-ADENOSYL-L-HOMOCYSTEINE | |
| | | | PD001319: V141-P276, K274-L332 | |
| | | | PUTATIVE ADENOSYL HOMOCYSTEINASE EC 3.3.1.1 S-ADENOSYL-L- | BLAST_PRODOM |
| | | | HOMOCYSTEINE HYDROLASE ADOHCYASE NAD ONE-CARBON | |
| | | | METABOLISM PD132567: K73-C140 | |
| | | | NAD DEHYDROGENASE OXEDOREDUCTASE HYDROLASE ADENOSYL | BLAST_PRODOM |
| | | | HOMOCYSTEINASE ADOHCYASE ONE-CARBON METABOLISM | |
| | | | PROTEIN S-ADENOSYL-L-HOMOCYSTEINE PD000699: D333-A441 | |
| | | | ADENOSYL HOMOCYSTEINASE HYDROLASE ADOHCYASE NAD ONE- | BLAST_PRODOM |
| | | | CARBON METABOLISM S-ADENOSYL-L-HOMOCYSTEINE PUTATIVE | |
| | | | S-ADENOSYL-L-HOMOCYSTEINE PD149849: M278-I331 | |
| | | | S-ADENOSYL-L-HOMOCYSTEINE HYDROLASE DM01437; | BLAST_DOMO |
| | | | \|P50245\|63-503: G135-R563; \|JC2480\|2-433: V141-Y564; | |
| | | | \|P27604\|3-436: V141-Y564; \|P35007\|9-484: K274-Y564, D138-T291 | |
| | | | Potential Phosphorylation Sites: S60 S104 S114 S118 S119 S124 S238 S330 | MOTIFS |
| | | | S434 S442 S455 T90 T216 T339 T374 T436 Y325 | |
| | | | Potential Glycosylation Sites: N54 N313 N478 | MOTIFS |
| 2 | 7485242CD1 | 178 | Signal_cleavage: M1-V42 | SPSCAN |
| | | | Signal Peptide: M23-M43, M23-D44, M23-K46 | HMMER |
| | | | Transmembrane domain: T28-N53; N is non-cytosolic | TMAP |
| | | | Pancreatic ribonuclease family proteins BL00127: G76-E120, I131-L174 | BLIMPS_BLOCKS |
| | | | Potential Phosphorylation Sites: S113 S161 | MOTIFS |
| | | | Potential Glycosylation Sites: N148 | MOTIFS |
| 3 | 2900469CD1 | 371 | Signal Peptide: M1-A34 | HMMER |
| | | | UbiA prenyltransferase family:; T86-L352 | HMMER_PFAM |
| | | | Transmembrane domains: F106-G126 G152-G172 C176-P196 T212-G240 P277- | TMAP |
| | | | S297 C329-K354; N-terminus is cytosolic | |
| | | | PROTEIN TRANSFERASE TRANSMEMBRANE 4HYDROXYBENZOATE | BLAST_PRODOM |
| | | | OXIDASE OCTAPRENYLTRANSFERASE CYTOCHROME C | |
| | | | BIOSYNTHESIS SYNTHASE; PD001657: T86-A306 | |
| | | | SIMILAR TO 4HYDROXYBENZOATE OCTAPRENYLTRANSFERASE | BLAST_PRODOM |
| | | | TRANSFERASE; PD124505: D255-I365 | |
| | | | HYDROXYBENZOATE; OCTAPRENYLTRANSFERASE; | BLAST_DOMO |
| | | | DM05150\|Q10252\|85-357: C93-K354; | |
| | | | DM05150\|P32378\|102-371: C93-L341; | |
| | | | DM05150\|P26601\|32-289: W95-W353 | |
| | | | Potential Phosphorylation Sites: S202 S335 T128 T142 T358 T361 | MOTIFS |
| | | | Potential Glycosylation Sites: N336 | MOTIFS |
| 4 | 6928818CD1 | 384 | Signal Peptide: M1-R35, M1-Q27 | HMMER |
| | | | Galactosyltransferase: E131-L365 | HMMER_PFAM |
| | | | Transmembrane domain: S8-S36; N-terminus is cytosolic. | TMAP |
| | | | TRANSFERASE GLYCOSYL-TRANSFERASE UDP-GAL: BETA-GLCNAC | BLAST_PRODOM |
| | | | BETA 1 PD004190: R132-A322 | |
| | | | Potential Phosphorylation Sites: | MOTIFS |
| | | | S36 S42 S125 S220 S324 S342 T10 T198 T291 Y95 | |
| | | | Potential Glycosylation Sites: N73 N77 N196 | MOTIFS |
| 5 | 1801591CD1 | 484 | FAD binding domain: E33-A234 | HMMER_PFAM |
| | | | PROTEIN OXIDASE SYNTHASE OXIDOREDUCTASE FLAVOPROTEIN | BLAST_PRODOM |
| | | | FAD DLACTATE DEHYDROGENASE GLYCOLATE SUBUNIT | |
| | | | PD002390: S255-L484; PD000960: V133-P254 | |
| | | | DEHYDROGENASE; GLCD; GLYCOLATE; OXIDASE; DM02882 | BLAST_DOMO |
| | | | \|S51528\|148-566: V70-V483; \|P32891\|155-575: V70-L484 | |
| | | | \|P52075\|61-471: P72-K482; \|P46681\|106-529: P72-L484 | |
| | | | Potential Phosphorylation Sites: S20 T99 T117 T139 T182 T236 T345 T382 | MOTIFS |
| | | | T464 | |
| | | | Potential Glycosylation Sites: N115 N217 N387 | MOTIFS |
| 6 | 2257558CD1 | 526 | ENDONUCLEASE tRNA SPLICING SUBUNIT PUTATIVE SEN54 tRNA | BLAST_PRODOM |
| | | | INTRON HYDROLASE NUCLEASE tRNA PROCESSING PD156270: L33- | |
| | | | P179, H431-I475 | |
| | | | Potential Phosphorylation Sites: S19 S30 S136 S178 S203 S204 S206 S281 | MOTIFS |
| | | | S349 S377 S393 S476 S487 S492 S514 T332 T460 Y119 | |
| 7 | 5701733CD1 | 567 | Signal Peptide: M1-A21, M1-K24 | HMMER |
| | | | Glycosyl hydrolase family 1: Y33-R507 | HMMER_PFAM |
| | | | Transmembrane domain: T8-Y34 S534-L562; | TMAP |
| | | | N-terminus is non-cytosolic | |
| | | | Glycosyl hydrolases family 1 proteins BL00572: | BLIMPS_BLOCKS |
| | | | F37-W66, D88-G121, N128-L162, I407-F418, D445-G472, R485-Y494 | |
| | | | Glycosyl hydrolases family 1 signatures | PROFILESCAN |
| | | | glycosyl_hydrol_f1_1: P388-N437; glycosyl_hydrol_f1_2: K24-G75 | |
| | | | Glycosyl hydrolase family 1 signature | BLIMPS_PRINTS |
| | | | PR00131: T335-I349, I407-S415, D425-I436, G446-W463, R470-N482 | |
| | | | HYDROLASE GLYCOSIDASE BETAGLUCOSIDASE PRECURSOR | BLAST_PRODOM |
| | | | CELLOBIASE SIGNAL AMYGDALASE GLUCOHYDROLASE | |
| | | | GENTIOBIASE CELLULOSE PD000650: T36-D365, S330-R507 | |
| | | | GLUCOSIDASE LIKE PROTEIN PD000648: D404-N501 | BLAST_PRODOM |
| | | | GLYCOSYL HYDROLASES FAMILY 1 N-TERMINAL DM00233; | BLAST_DOMO |
| | | | \|P09848\|1368-1838: E29-G502; \|JS0610\|1369-1839: E29-G502; | |
| | | | \|P09848\|899-1366: Y33-P504; \|JS0610\|901-1367: Y33-P504 | |
| | | | Glycosyl hydrolases family 1 signatures: I407-S415, T41-A55 | MOTIFS |
| | | | Potential Phosphorylation Sites: S26 S64 S72 S325 S415 S456 S468 S567 T198 | MOTIFS |
| | | | T247 T345 T350 Y210 Y354 Y467 Y513 | |
| | | | Potential Glycosylation Sites: N80 N171 N245 | MOTIFS |
| 8 | 2706884CD1 | 318 | Signal Cleavage: M1-D17 | SPSCAN |
| | | | Signal Peptide: M1-Q20 | HMMER |
| | | | Eukaryotic-type carbonic anhydrase: W22-F278 | HMMER_PFAM |
| | | | Eukaryotic-type carbonic anhydrase BL00162: W33-A63, Q71-T93, V104- | BLIMPS_BLOCKS |
| | | | D140, S143-G167, Q208-Q240, Q245-F278 | |
| | | | Eukaryotic-type carbonic anhydrases signature | PROFILESCAN |
| | | | euk_co2_anhydrase: G97-A158 | |
| | | | CARBONIC ANHYDRASE DEHYDRATASE LYASE CARBONATE ZINC | BLAST_PRODOM |
| | | | PRECURSOR SIGNAL PROTEIN GLYCOPROTEIN | |
| | | | PD000865: H21-F278 | |
| | | | CARBONIC ANHYDRASE DM00356 | BLAST_DOMO |
| | | | \|I38013\|157-390: Q45-F278 \|P08060\|26-260: G41-S277 | |
| | | | \|P23280\|43-277: G41-V274 \|P48283\|44-280: C40-S277 | |
| | | | Potential Phosphorylation Sites: | MOTIFS |
| | | | S36 S46 S146 S250 T130 T251 T286 T293 | |
| | | | Potential Glycosylation Sites: N213 | MOTIFS |
| 9 | 4974616CD1 | 556 | Signal Peptide: M1-C34 | SPSCAN |
| | | | Glycosyl transferases: S118-F302 | HMMER_PFAM |
| | | | Similarity to lectin domain of ricin: R425-R550 | HMMER_PFAM |
| | | | Transmembrane segments: F4-F28; N-terminus non-cytosolic | TMAP |
| | | | Gtycosyltransferase PF0535: I151-F161, S199-D208 | BLMPS_PFAM |
| | | | N-ACETYLGALACTOSAMINYLTRANSFERASE TRANSFERASE | BLAST_PRODOM |
| | | | PD003162: R265-P424; PD003677: E55-T117 | |
| | | | PD013169: M1-A45; PD000196: N116-F264 | |
| | | | ACETYLGALACTOSAMINYLTRANSFERASE; POLYPEPTIDE; DM03891 | BLAST_DOMO |
| | | | Q07537\|32-558: N32-L554; P34678\|37-600: D36-L549; | |
| | | | I37405\|21-571: N81-W547 | |
| | | | Potential Phosphorylation Sites: | MOTIFS |
| | | | S96 S157 S323 S396 S429 S527 T205 T349 T400 T513 T535 | |
| | | | Potential Glycosylation Sites: N94 N116 N551 | MOTIFS |
| 10 | 70861047CD1 | 598 | Signal Peptide: M1-C28 | SPSCAN |
| | | | Signal Peptide: M1-A26 | HMMER |
| | | | Glycosyl transferees: S155-G341 | HMMER_PFAM |
| | | | Transmembrane segments: L4-R29 L145-S171; N-terminus non-cytosolic | TMAP |
| | | | N-ACETYLGALACTOSAMINYLTRANSFERASE | BLAST_PRODOM |
| | | | PD003162: D315-M457 | |
| | | | ACETYLGALACTOSAMINYLTRANSFERASE; POLYPEPTIDE; DM0389 | BLAST_DOMO |
| | | | Q07537\|32-558: G93-N595; P34678\|37-600: A56-L569; | |
| | | | I37405\|21-571: P114-W591 | |
| | | | Potential Phosphorylation Sites: | MOTIFS |
| | | | S3 S68 S104 S125 S133 S194 S432 S440 S596 T394 T565 T592 Y120 Y147 | |
| | | | Potential Glycosylation Sites: N50 N461 N486 | MOTIFS |
| 11 | 7472794CD1 | 230 | Acetyltransferase (GNAT) family: E138-E208 | HMMER_PFAM |
| | | | PUTATIVE GLYCINENACYLTRANSFERASE ARALKYL ACYLCOA: | BLAST_PRODOM |
| | | | AMINO ACID GLYCINE ARYLACETYL PD022048: L2-A124 | |
| | | | NACYLTRANSFERASE TRANSFERASE ACYLTRANSFERASE ARALKYL | BLAST_PRODOM |
| | | | ACYLCOA: AMINO ACID GLYCINE ARYLACETYL ACYLCOA | |
| | | | ARYLACETYLTRANSFERASE PD034577: R95-P211 | |
| | | | Potential Phosphorylation Sites: S9 S33 S93 S146 T42 T159 | MOTIFS |
| | | | Potential Glycosylation Sites: N108 | MOTIFS |

TABLE 4

| Polynucleotide | |
|---|---|
| SEQ ID NO:/ | |
| Incyte ID/Sequence | |
| Length | Sequence Fragments |
| 12/7484737CB1/1891 | 1-560, 228-1037, 232-869, 233-643, 233-1041, 234-1041, 274-847, 274-992, 307-1042, 331-830, 335-1042, 358- |
| | 1042, 423-1042, 500-757, 510-850, 519-1041, 551-1260, 588-829, 692-1042, 764-1041, 1002-1154, 1026-1631, |
| | 1063-1694, 1125-1161, 1125-1766, 1125-1836, 1125-1868, 1125-1890, 1126-1761, 1126-1855, 1127-1869, 1131- |
| | 1679, 1131-1766, 1154-1853, 1215-1855, 1224-1400, 1224-1544, 1224-1571, 1224-1578, 1224-1619, 1224-1636, |
| | 1224-1670, 1224-1681, 1224-1721, 1224-1747, 1224-1872, 1225-1630, 1238-1579, 1258-1870, 1263-1717, 1274- |
| | 1826, 1289-1766, 1320-1420, 1322-1532, 1388-1522, 1431-1874, 1462-1861, 1462-1862, 1472-1861, 1474-1855, |
| | 1503-1863, 1557-1861, 1565-1690, 1610-1891, 1626-1875, 1668-1861, 1675-1861, 1696-1861, 1731-1874, 1803- |
| | 1863 |
| 13/7485242CB1/1056 | 1-556, 1-634, 1-660, 98-546, 98-616, 98-645, 98-660, 586-1056, 607-999 |
| 14/2900469CB1/1520 | 1-261, 1-676, 7-638, 226-474, 257-497, 263-588, 263-810, 312-562, 330-904, 331-691, 386-684, 405-629, 483-797, |
| | 551-839, 551-979, 551-1098, 621-1161, 621-1287, 644-972, 685-903, 685-958, 685-1223, 687-1177, 701-1012, 712- |
| | 1131, 748-1470, 768-1055, 768-1070, 803-1062, 827-1020, 874-1497, 882-1492, 887-1509, 972-1446, 1033-1501, |
| | 1037-1502, 1046-1515, 1049-1520, 1054-1509, 1057-1520, 1085-1269, 1095-1509, 1111-1304, 1114-1508, 1115- |
| | 1509, 1140-1520, 1141-1491, 1143-1518, 1157-1507, 1174-1511, 1265-1518 |
| 15/6928818CB1/3007 | 1-797, 443-899, 443-948, 443-1006, 443-1122, 443-1230, 443-1232, 443-1236, 443-1243, 443-1247, 443-1253, 448- |
| | 1176, 450-1122, 572-1310, 606-1428, 617-1428, 639-1428, 666-1366, 756-1563, 807-1366, 847-1368, 849-1535, |
| | 882-1679, 885-1673, 985-1318, 985-1338, 985-1572, 996-1569, 1041-1865, 1072-1211, 1081-1697, 1081-1699, |
| | 1108-1969, 1177-1985, 1186-1542, 1197-1542, 1252-2113, 1263-2090, 1277-1966, 1293-1713, 1297-1720, 1313- |
| | 2155, 1345-2231, 1361-2189, 1382-2283, 1396-2200, 1426-2283, 1428-2283, 1430-2283, 1432-2283, 1442-2283, |
| | 1449-1872, 1476-1900, 1478-1905, 1478-2280, 1480-2283, 1484-2283, 1528-2220, 1530-1999, 1600-1928, 1600- |
| | 2278, 1655-1698, 1686-1825, 1687-1825, 1877-2542, 1906-1989, 1929-2257, 1932-2012, 2088-2479, 2088-2492, |
| | 2092-2492, 2120-2491, 2275-2969, 2276-2969, 2276-2970, 2281-2969, 2284-3005, 2297-2505, 2317-2529, 2317- |
| | 2655, 2317-2732, 2317-2754, 2317-2786, 2317-2797, 2317-2820, 2317-2821, 2317-2882, 2317-2960, 2317-3007, |
| | 2324-2594, 2486-2821, 2493-2689, 2804-2882 |
| 16/1801591CB1/2058 | 1-234, 57-537, 62-296, 62-320, 62-618, 72-325, 72-649, 87-233, 90-504, 183-425, 184-695, 184-735, 190-691, 357- |
| | 602, 469-871, 529-871, 695-972, 815-1473, 819-1121, 893-1468, 917-1189, 917-1475, 962-1153, 962-1253, 1005- |
| | 1668, 1022-1153, 1045-1285, 1112-1391, 1131-1637, 1131-1677, 1153-1299, 1166-1785, 1178-1563, 1198-1395, |
| | 1207-1817, 1224-1483, 1224-1752, 1241-1495, 1242-1524, 1269-1556, 1272-1819, 1281-1673, 1308-1556, 1319- |
| | 1749, 1335-1627, 1347-1902, 1354-1959, 1372-1747, 1372-1753, 1373-1641, 1392-1957, 1408-2001, 1415-1678, |
| | 1427-1891, 1482-1793, 1485-1970, 1497-1812, 1506-1803, 1509-1781, 1536-1978, 1592-2017, 1602-2058, 1690- |
| | 1941 |
| 17/2257558CB1/1951 | 1-166, 1-291, 16-468, 21-549, 21-565, 27-285, 31-236, 33-568, 35-282, 36-85, 69-297, 251-445, 275-497, 316-825, |
| | 341-1006, 477-948, 483-1032, 620-865, 620-1095, 663-1227, 671-903, 674-1016, 698-823, 732-1048, 756-1025, |
| | 825-1442, 841-1057, 874-1151, 875-1161, 912-1123, 912-1281, 916-1532, 1000-1297, 1048-1332, 1048-1591, 1076- |
| | 1504, 1083-1560, 1125-1342, 1131-1594, 1342-1916, 1355-1919, 1392-1651, 1406-1883, 1416-1951, 1428-1711, |
| | 1428-1742, 1428-1901, 1428-1932, 1428-1947, 1435-1893, 1525-1781, 1554-1854, 1574-1805, 1635-1874, 1635- |
| | 1892, 1635-1951, 1636-1890, 1708-1927, 1711-1902, 1714-1951, 1782-1951 |
| 18/5701733CB1/2266 | 1-114, 43-114, 73-345, 77-456, 115-180, 115-184, 115-185, 115-293, 115-467, 115-553, 115-611, 115-692, 115- |
| | 724, 115-799, 115-806, 115-807, 115-821, 115-828, 115-855, 116-814, 116-853, 126-923, 151-1025, 164-850, 175- |
| | 1018, 226-779, 296-1136, 306-954, 392-1167, 401-986, 401-1271, 480-1129, 523-1048, 533-1315, 533-1316, 533- |
| | 1326, 533-1332, 533-1334, 533-1343, 539-1374, 540-1300, 564-1076, 583-1386, 609-1146, 612-1146, 616-1175, |
| | 650-1548, 652-1092, 655-956, 680-1332, 719-1547, 785-1441, 792-1618, 817-1482, 836-1635, 891-1436, 902-1508, |
| | 903-1828, 973-1747, 977-1533, 990-1503, 1065-1552, 1220-1981, 1256-1981, 1659-1724, 1725-1821, 1725-1829, |
| | 1725-1837, 1725-1848, 1725-1902, 1725-1906, 1725-1956, 1725-1960, 1725-1991, 1725-1998, 1725-2147, 1739- |
| | 1873, 1745-2120, 1783-2010, 1784-2266, 1798-2074, 1800-2086, 1845-2266, 1860-2153 |
| 19/2706884CB1/1657 | 1-196, 1-316, 1-535, 36-384, 42-116, 51-226, 53-300, 53-379, 55-560, 57-515, 59-539, 60-488, 64-161, 74-315, 80- |
| | 387, 84-467, 95-340, 99-595, 242-519, 243-445, 243-752, 282-566, 292-599, 361-856, 557-704, 561-849, 726-961, |
| | 726-1165, 743-1630, 766-1036, 766-1038, 768-867, 828-867, 866-1025, 866-1366, 1025-1165, 1097-1395, 1150- |
| | 1657, 1164-1217, 1164-1332, 1164-1350, 1208-1474 |
| 20/4974616CB1/2331 | 1-485, 423-570, 539-865, 539-1005, 540-822, 638-873, 638-1052, 696-928, 762-1129, 864-1410, 1067-1600, 1121- |
| | 1743, 1141-1761, 1453-2111, 1560-2029, 1561-1812, 1753-2005, 1753-2017, 1758-2331, 1768-2303, 1787-2166 |
| 21/70861047CB1/3439 | 1-677, 174-809, 174-831, 174-845, 459-1011, 764-1391, 784-1457, 798-1457, 854-1443, 978-1244, 978-1716, 1102- |
| | 1591, 1335-1869, 1417-1679, 1440-1941, 1463-1607, 1576-1768, 1603-2137, 1672-2175, 1718-1987, 1742-2193, |
| | 1767-2316, 1801-2388, 1824-2084, 1841-2298, 1861-2104, 1909-2527, 1930-2520, 1948-2441, 1976-2247, 1976- |
| | 2330, 2001-2538, 2069-2521, 2092-2313, 2092-2665, 2201-2474, 2206-2504, 2224-2448, 2235-2489, 2240-2531, |
| | 2240-2720, 2261-2511, 2294-2867, 2295-2682, 2297-2569, 2300-2472, 2327-2605, 2356-2584, 2359-2642, 2407- |
| | 2855, 2409-2628, 2409-2696, 2451-3001, 2459-2680, 2480-2717, 2480-2989, 2487-2749, 2520-2765, 2663-2952, |
| | 2681-2967, 2706-2973, 2739-3428, 2771-3006, 2771-3249, 2780-3430, 2843-3099, 2847-3437, 2875-3410, 2889- |
| | 3191, 2905-3435, 2908-3119, 2954-3370, 3130-3438, 3130-3439, 3131-3340 |
| 22/7472794CB1/2749 | 1-440, 28-62, 28-69, 28-73, 28-115, 28-143, 28-194, 28-232, 28-312, 28-313, 28-342, 28-344, 28-377, 28-382, 28- |
| | 403, 28-419, 28-434, 28-438, 28-463, 28-467, 28-517, 28-544, 28-608, 28-773, 29-605, 31-773, 32-777, 34-115, 34- |
| | 608, 34-773, 35-778, 71-608, 72-778, 80-608, 81-608, 86-778, 90-608, 120-608, 193-608, 223-767, 223-769, 223- |
| | 772, 223-778, 245-608, 252-778, 336-548, 389-608, 391-608, 421-607, 421-608, 422-608, 437-608, 458-595, 458- |
| | 608, 605-773, 605-778, 640-1002, 645-1002, 890-1017, 892-1017, 901-1017, 1018-1443, 1033-1443, 1066-1439, |
| | 1370-2033, 1371-1745, 1371-1794, 1371-1808, 1371-1853, 1371-1875, 1371-1916, 1371-1948, 1371-1991, 1371- |
| | 2028, 1371-2086, 1372-1686, 1372-2086, 1376-2040, 1376-2086, 1383-2086, 1384-2004, 1385-2086, 1472-2044, |
| | 1546-2203, 1574-2018, 1578-2165, 1587-2114, 1665-1686, 1665-1691, 1665-2283, 1665-2284, 1665-2351, 1665- |
| | 2374, 1665-2380, 1665-2390, 1665-2405, 1665-2409, 1670-2488, 1683-1839, 1686-2515, 1778-2353, 1799-2434, |
| | 1871-2438, 1895-2579, 1908-2744, 1914-2579, 1950-2748, 1952-2744, 1955-2749, 1961-2579, 1964-2576, |
| | 1969-2744, 1972-2749, 1998-2411, 2001-2579, 2042-2579, 2043-2749, 2051-2579, 2052-2579, 2057-2749, 2061- |
| | 2579, 2091-2579, 2164-2579, 2194-2738, 2194-2740, 2194-2743, 2194-2749, 2216-2579, 2223-2749, 2307-2519, |
| | 2360-2579, 2362-2579, 2392-2578, 2392-2579, 2393-2579, 2408-2579, 2429-2566, 2429-2579, 2576-2744, 2576- |
| | 2749, 2611-2749, 2616-2749 |

TABLE 5

| Polynucleotide SEQ | | |
|---|---|---|
| ID NO: | Incyte Project ID: | Representative Library |
| 12 | 7484737CB1 | DRGTNON04 |
| 14 | 2900469CB1 | LVENNOT03 |
| 15 | 6928818CB1 | ESOGTUE01 |
| 16 | 1801591CB1 | PROSTUS23 |
| 17 | 2257558CB1 | LUNGNOT10 |
| 18 | 5701733CB1 | KIDNTUT01 |
| 19 | 2706884CB1 | PONSAZT01 |
| 20 | 4974616CB1 | HNT2AGT01 |
| 21 | 70861047CB1 | BRAXTDR12 |
| 22 | 7472794CB1 | COLNTUN03 |

TABLE 6

| Library | Vector | Library Description |
|---|---|---|
| BRAXTDR12 | PCDNA2.1 | This random primed library was constructed using RNA isolated from frontal neocortex |
| | | tissue removed from a 55-year-old Caucasian female who died from cholangiocarcinoma. |
| | | Pathology indicated mild meningeal fibrosis predominately over the convexities, |
| | | scattered axonal spheroids in the white matter of the cingulate cortex and the |
| | | thalamus, and a few scattered neurofibrillary tangles in the entorhinal cortex and |
| | | the periaqueductal gray region. Pathology for the associated tumor tissue indicated |
| | | well-differentiated cholangiocarcinoma of the liver with residual or relapsed tumor. |
| | | Patient history included cholangiocarcinoma, post-operative Budd-Chiari syndrome, |
| | | biliary ascites, hydrothorax, dehydration, malnutrition, oliguria and acute renal |
| | | failure. Previous surgeries included cholecystectomy and resection of 85% of the |
| | | liver. |
| COLNTUN03 | pINCY | This normalized pooled colon tumor tissue library was constructed from 1.16 million |
| | | independent clones from a pooled colon tumor library. Starting library was |
| | | constructed using pooled cDNA from 6 donors. cDNA was generated using mRNA isolated |
| | | from colon tumor tissue removed from a 55-year-old Caucasian male (A) during |
| | | hemicolectomy; from a 60 year-old Caucasian male (B) during hemicolectomy; from a |
| | | 62-year-old Caucasian male (C) during sigmoidectomy; from a 30-year-old Caucasian |
| | | female (D) during hemicolectomy; from a 64-year-old Caucasian female (E) during |
| | | hemicolectomy; and from a 70-year-old Caucasian female (F) during hemicolectomy. |
| | | Pathology indicated invasive grade 3 adenocarcinoma (A); invasive grade 2 |
| | | adenocarcinoma (B); invasive grade 2 adenocarcinoma (C); carcinoid tumor (D); |
| | | invasive grade 3 adenocarcinoma (E); and invasive grade 2 adenocarcinoma (F). Donors |
| | | B, C, D, E, and F had positive lymph nodes. Patient medications included Ativan (A); |
| | | Seldane (B), Tri-Levlen (D); Synthroid (E); Tamoxifen, prednisone, Synthroid, and |
| | | Glipizide (F). The library was normalized in two rounds using conditions adapted |
| | | from Scares et al., PNAS (1994) 91:9 |
| DRGTNON04 | pINCY | The normalized dorsal root ganglion tissue library was constructed from 5.64 million |
| | | independent clones from the a dorsal root ganglion library. Starting RNA was made |
| | | from thoracic dorsal root ganglion tissue from a 32-year-old Caucasian male, who died |
| | | from acute pulmonary edema, acute bronchopneumonia, pleural and pericardial effusion, |
| | | and lymphoma. The patient presented with pyrexia, fatigue, and GI bleeding. Patient |
| | | history included probable cytomegalovirus infection, liver congestion and steatosis, |
| | | splenomegaly, hemorrhagic cystitis, thyroid hemorrhage, respiratory failure, |
| | | pneumonia, natural killer cell lymphoma of the pharynx, Bell'spalsy, and tobacco and |
| | | alcohol abuse. The library was normalized in one round using conditions adapted from |
| | | Soares et al., PNAS (1994) 91:9228 and Bonaldo et al., Genome Research 6 (1996):791, |
| | | except that a significantly longer (48-hours/round) reannealing hybridization was |
| | | used. The library was then linearized and recircularized to select for insert |
| | | containing clones as follows: plasmid DNA was prepped from approximately 1 million |
| | | clones from the normalized dorsal root ganglion tissue library following soft agar |
| | | transformation. |
| ESOGTUE01 | pINCY | This 5′ biased random primed library was constructed using RNA isolated from |
| | | esophageal tumor tissue removed from a 61 year-old Caucasian male during a partial |
| | | esophagectomy, proximal gastrectomy, pyloromyotomy, and regional lymph node excision. |
| | | Pathology indicated an invasive grade 3 adenocarcinoma in the esophagus, extending |
| | | distally to involve the gastroesophageal junction. The tumor extended through the |
| | | muscularis to involve periesophageal and perigastric soft tissues. One perigastric |
| | | and two periesophageal lymph nodes were positive for tumor. There were multiple |
| | | perigastric and periesophageal tumor implants. The patient presented with deficiency |
| | | anemia and myelodysplasia. Patient history included hyperlipidemia, and tobacco and |
| | | alcohol abuse in remission. Previous surgeries included adenotonsillectomy, |
| | | rhinoplasty, vasectomy, and hemorrhoidectomy. A previous bone marrow aspiration found |
| | | the marrow to be hypercellular for age and had a cellularity-to-fat ratio of 95:5. |
| | | The marrow was focally densely fibrotic. Granulocytic precursors were slightly |
| | | increased with normal maturation. The estimate of blast cells was greater than 5%. |
| | | Megakaryocytes were increased and app |
| HNT2AGT01 | PBLUESCRIPT | Library was constructed at Stratagene (STR937233), using RNA isolated from the hNT2 |
| | | cell line derived from a human teratocarcinoma that exhibited properties |
| | | characteristic of a committed neuronal precursor. Cells were treated with retinoic |
| | | acid for 5 weeks and with mitotic inhibitors for two weeks and allowed to mature for |
| | | an additional 4 weeks in conditioned medium. |
| KIDNTUT01 | PSPORT1 | Library was constructed using RNA isolated from the kidney tumor tissue removed from |
| | | an 8-month-old female during nephroureterectomy. Pathology indicated Wilms' tumor |
| | | (nephroblastoma), which involved 90 percent of the renal parenchyma. Prior to |
| | | surgery, the patient was receiving heparin anticoagulant therapy. |
| LUNGNOT10 | pINCY | Library was constructed using RNA isolated from the lung tissue of a Caucasian male |
| | | fetus, who died at 23 weeks' gestation. |
| LVENNOT03 | PSPORT1 | Library was constructed using RNA isolated from the left ventricle tissue of a |
| | | 31-year-old male. |
| PONSAZT01 | pINCY | Library was constructed using RNA isolated from diseased pons tissue removed from |
| | | the brain of a 74-year-old Caucasian male who died from Alzheimer's disease. |
| PROSTUS23 | pINCY | This subtracted prostate tumor library was constructed using 10 million clones from |
| | | a pooled prostate tumor library that was subjected to 2 rounds of subtractive |
| | | hybridization with 10 million clones from a pooled prostate tissue library. The |
| | | starting library for subtraction was constructed by pooling equal numbers of clones |
| | | from 4 prostate tumor libraries using mRNA isolated from prostate tumor removed from |
| | | Caucasian males at ages 58 (A), 61 (B), 66 (C), and 68 (D) during prostatectomy with |
| | | lymph node excision. Pathology indicated adenocarcinoma in all donors. History |
| | | included elevated PSA, induration and tobacco abuse in donor A; elevated PSA, |
| | | induration, prostate hyperplasia, renal failure, osteoarthritis, renal artery |
| | | stenosis, benign HTN, thrombocytopenia, hyperlipidemia, tobacco/alcohol abuse and |
| | | hepatitis C (carrier) in donor B; elevated PSA, induration, and tobacco abuse in |
| | | donor C; and elevated PSA, induration, hypercholesterolemia, and kidney calculus in |
| | | donor D. The hybridization probe for subtraction was constructed by pooling equal |
| | | numbers of cDNA clones from 3 prostate tissue libraries derived from prostate tissue, |
| | | prostate epithelial cells, and fibroblasts from prostate str [symbol] |

TABLE 7

| | | | Parameter |
|---|---|---|---|
| Program | Description | Reference | Threshold |
| ABI | A program that removes vector sequences and | Applied Biosystems, Foster City, CA. | |
| FACTURA | masks ambiguous bases in nucleic acid sequences. | | |
| ABI/ | A Fast Data Finder useful in comparing and | Applied Biosystems, Foster City, CA; | Mismatch <50% |
| PARACEL | annotating amino acid or nucleic acid sequences. | Paracel Inc., Pasadena, CA. | |
| FDF | | | |
| ABI | A program that assembles nucleic acid sequences. | Applied Biosystems, Foster City, CA. | |
| Auto- | | | |
| Assembler | | | |
| BLAST | A Basic Local Alignment Search Tool useful in | Altschul, S. F. et al. (1990) J. Mol. Biol. | ESTs: Probability |
| | sequence similarity search for amino acid and | 215: 403-410; Altschul, S. F. et al. (1997) | value = 1.0E−8 |
| | nucleic acid sequences. BLAST includes five | Nucleic Acids Res. 25: 3389-3402. | or less; Full |
| | functions: blastp, blastn, blastx, tblastn, | | Length sequences: |
| | and tblastx. | | Probability value = |
| | | | 1.0E−10 or less |
| FASTA | A Pearson and Lipman algorithm that searches | Pearson, W. R. and D. J. Lipman (1988) Proc. | ESTs: fasta E |
| | for similarity between a query sequence and a | Natl. Acad Sci. USA 85: 2444-2448; Pearson, | value = 1.06E−6; |
| | group of sequences of the same type. FASTA | W. R. (1990) Methods Enzymol. 183: 63-98; | Assembled ESTs: |
| | comprises as least five functions: fasta, | and Smith, T. F. and M. S. Waterman (1981) | fasta Identity = |
| | tfasta, fastx, tfastx, and ssearch. | Adv. Appl. Math. 2: 482-489. | 95% or greater |
| | | | and Match |
| | | | length = 200 |
| | | | bases or greater; |
| | | | fastx E value = |
| | | | 1.0E−8 or less; |
| | | | Full Length |
| | | | sequences: fastx |
| | | | score = 100 |
| | | | or greater |
| BLIMPS | A BLocks IMProved Searcher that matches a | Henikoff, S. and J. G. Henikoff (1991) | Probability value = |
| | sequence against those in BLOCKS, PRINTS, | Nucleic Acids Res. 19: 6565-6572; Henikoff, | 1.0E−3 or less |
| | DOMO, PRODOM, and PFAM databases to search | J. G. and S. Henikoff (1996) Methods | |
| | for gene families, sequence homology, and | Enzymol. 266: 88-105; and Attwood, T. K. et | |
| | structural fingerprint regions. | al. (1997) J. Chem. Inf. Comput. Sci. 37: 417- | |
| | | 424. | |
| HMMER | An algorithm for searching a query sequence | Krogh, A. et al. (1994) J. Mol. Biol. | PFAM or SMART |
| | against hidden Markov model (HMM)-based | 235: 1501-1531; Sonnhammer, E. L. L. et al. | hits: Probability |
| | databases of protein family consensus | (1988) Nucleic Acids Res. 26: 320-322; | value = 1.0E−3 |
| | sequences, such as PFAM and SMART. | Durbin, R. et al. (1998) Our World View, in | or less; Signal |
| | | a Nutshell, Cambridge Univ. Press, pp. 1- | peptide hits: |
| | | 350. | Score = 0 |
| | | | or greater |
| Pro- | An algorithm that searches for structural and | Gribskov, M. et al. (1988) CABIOS 4: 61-66; | Normalized quality |
| fileScan | sequence motifs in protein sequences that match | Gribskov, M. et al. (1989) Methods | score ≧ GCG- |
| | sequence patterns defined in Prosite. | Enzymol. 183: 146-159; Bairoch, A. et al. | specified “HIGH” |
| | | (1997) Nucleic Acids Res. 25: 217-221. | value for that |
| | | | particular Prosite |
| | | | motif. Generally, |
| | | | score = 1.4-2.1. |
| Phred | A base-calling algorithm that examines | Ewing, B. et al. (1998) Genome Res. 8: 175- | |
| | automated sequencer traces with high | 185; Ewing, B. and P. Green (1998) Genome | |
| | sensitivity and probability. | Res. 8: 186-194. | |
| Phrap | A Phils Revised Assembly Program including | Smith, T. F. and M. S. Waterman (1981) Adv. | Score = 120 or |
| | SWAT and CrossMatch, programs based on | Appl. Math. 2: 482-489; Smith, T. F. and | greater; Match |
| | efficient implementation of the Smith-Waterman | M. S. Waterman (1981) J. Mol. Biol. 147: 195- | length = |
| | algorithm, useful in searching sequence | 197; and Green, P., University of | 56 or greater |
| | homology and assembling DNA sequences. | Washington, Seattle, WA. | |
| Consed | A graphical tool for viewing and editing Phrap | Gordon, D. et al. (1998) Genome Res. 8: 195- | |
| | assemblies. | 202. | |
| SPScan | A weight matrix analysis program that scans | Nielson, H. et al. (1997) Protein Engineering | Score = 3.5 |
| | protein sequences for the presence of secretory | 10: 1-6; Claverie, J. M. and S. Audic (1997) | or greater |
| | signal peptides. | CABIOS 12: 431-439. | |
| TMAP | A program that uses weight matrices to | Persson, B. and P. Argos (1994) J. Mol. Biol. | |
| | delineate transmembrane segments on protein | 237: 182-192; Persson, B. and P. Argos | |
| | sequences and determine orientation. | (1996) Protein Sci. 5: 363-371. | |
| TMHMMER | A program that uses a hidden Markov model (HMM) | Sonnhammer, E. L. et al. (1998) Proc. Sixth | |
| | to delineate transmembrane segments on protein | Intl. Conf. On Intelligent Systems for Mol. | |
| | sequences and determine orientation. | Biol., Glasgow et al., eds., The Am. Assoc. | |
| | | for Artificial Intelligence (AAAI) Press, | |
| | | Menlo Park, CA, and MIT Press, Cambridge, | |
| | | MA, pp. 175-182. | |
| Motifs | A program that searches amino acid sequences for | Bairoch, A. et al. (1997) Nucleic Acids Res. | |
| | patterns that matched those defined in Prosite. | 25: 217-221; Wisconsin Package Program | |
| | | Manual, version 9, page M51-59, Genetics | |
| | | Computer Group, Madison, WI. | |

1. An isolated polypeptide selected from the group consisting of:
a) a polypeptide comprising an amino acid sequence selected from the group consisting of SEQ ID NO:1-11,
b) a polypeptide comprising a naturally occurring amino acid sequence at least 90% identical to an amino acid sequence selected from the group consisting of SEQ ID NO:1-11,
c) a biologically active fragment of a polypeptide having an amino acid sequence selected from the group consisting of SEQ ID NO:1-11, and
d) an immunogenic fragment of a polypeptide having an amino acid sequence selected from the group consisting of SEQ ID NO:1-11.

2. An isolated polypeptide of claim 1 comprising an amino acid sequence selected from the group consisting of SEQ ID NO:1-11.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:12-22.

6. A recombinant polynucleotide comprising a promoter sequence operably linked to a polynucleotide of claim 3.

7. A cell transformed with a recombinant polynucleotide of claim 6.

8. (CANCELED)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-11.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:12-22,
b) a polynucleotide comprising a naturally occurring polynucleotide sequence at least 90% identical to a polynucleotide sequence selected from the group consisting of SEQ ID NO:12-22,
c) a polynucleotide complementary to a polynucleotide of a),
d) a polynucleotide complementary to a polynucleotide of b), and
e) an RNA equivalent of a)-d).

13. (CANCELED)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (CANCELED)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-11.

19. (CANCELED)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. (CANCELED)

22. (CANCELED)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. (CANCELED)

25. (CANCELED)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (CANCELED)

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-77. (CANCELED)
